(19) United States
(12) Patent Application Publication
(10) Pub. No.: US 2024/0354446 A1
(43) Pub. Date: Oct. 24, 2024

(54) PRIVACY-BASED MONITORING SYSTEM AND METHOD

(71) Applicant: SPECTRONIX INC., Gatineau (CA)

(72) Inventor: Patrick Assouad, Ottawa (CA)

(21) Appl. No.: 18/639,499

(22) Filed: Apr. 18, 2024

Related U.S. Application Data
(63) Continuation-in-part of application No. PCT/CA2023/050288, filed on Mar. 6, 2023.
(60) Provisional application No. 63/316,697, filed on Mar. 4, 2022.
(60) Provisional application No. 63/536,368, filed on Sep. 1, 2023.

(51) Int. Cl. G06F 21/62; G16H 40/67

(52) U.S. Cl. CPC G06F 21/6254; G16H 40/67

(57) ABSTRACT

Described are various embodiments of a privacy-based monitoring system and method, including a system for automatically monitoring one or more individuals in a designated environment whilst maintaining the privacy of the one or more individuals by automatically anonymizing the data acquired that is not inherently anonymized. Use-cases are varied and range from detecting self-harm events, to harm events between individuals, and to other security risk events applicable in security surveillance applications.

### FIELD OF THE DISCLOSURE

The present disclosure relates to remote sensing, and, in particular, to a system for automatically monitoring one or more individuals in a designated environment whilst maintaining privacy of the one or more individuals, and to a related method.

### BACKGROUND

Signs that a prisoner may be at risk of suicide include giving away valued possessions, speaking as if they are not going to be around much longer, even though they are not scheduled for release, withdrawing, becoming acutely intoxicated, having a recent history of severe addiction, being threatened or assaulted by other prisoners, having a history of psychiatric hospitalizations or suicide attempts, talking about death, having recently been arrested for an offense punishable by a long sentence or actually sentenced to a lengthy term, or having impulse-control problems. Failure to consider obvious and substantial risk factors in assessing a potential for such self-harm is of concern.

Similar considerations may apply in other institutional or long-term care settings, for example, in a psychiatric ward or hospital, an old age or retirement home, a rehabilitation centre, or the like. Indeed, similar considerations may be applicable in detecting the occurrence of accidental harm events in these settings.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for a system for automatically monitoring one or more individuals in a designated environment whilst maintaining privacy of the one or more individuals and a related method that overcome some of the drawbacks of known techniques, or at least, provides a useful alternative thereto. Some aspects of this disclosure provide examples of such systems and methods.

In accordance with one aspect of the disclosure, there is provided a system for automatically monitoring an individual in a designated environment to identify a risk of harm while maintaining a privacy of the individual, the system comprising: an image-based sensor capturing image-based data of the designated environment to extract therefrom a digital representation of a posture of the individual within the designated environment such that an identify of the individual is unidentifiable from said digital representation; a radar capturing radar data of the designated environment; a network communication interface; a computer-readable medium having stored thereon characteristic features of multiple anticipated harm scenarios, wherein at least some of said anticipated harm scenarios are digitally characterised by a respective posture and vital sign; a digital data processor executing digital instructions in real-time to digitally: process said radar data to extract a current vital sign of the individual being monitored; compare said digital representation of the posture with multiple predefined postures to categorize a current predefined posture of the individual being monitored; identify a risk of harm upon said current vital sign and said current predefined posture corresponding with one of said anticipated harm scenarios; and communicate an alert corresponding to said identified risk of harm via said control network communication interface.

In one embodiment, the image-based sensor comprises a depth-enabled image sensor.

In one embodiment, the image-based sensor captures sequential image-based data over time to extract therefrom a posture sequence representative of a gesture, wherein at least some of said anticipated harm scenarios are digitally characterised by a respective gesture and vital sign, and wherein said digital data processor executes digital instructions in real-time to digitally compare said digital representation of said gesture with multiple predefined gestures to categorize a current predefined gesture of the individual being monitored to identify a risk of harm upon said current vital sign and said current predefined gesture corresponding with one of said anticipated harm scenarios.

In one embodiment, the digital representation of said posture comprises a digital representation of a physical disposition of physical body parts of the individual being monitored relative to said designated environment.

In one embodiment, at least one of said anticipated harm scenarios is characterised by a predefined physical disposition defined by a suspended vertical body orientation.

In one embodiment, the vital sign comprises at least one of a heart rate and a respiration rate.

In one embodiment, the image-based data is digitally anonymized by digitally removing predefined personally identifiable information (PII).

In one embodiment, the image-based data is anonymized by executing a predefined data anonymization process.

In one embodiment, the predefined data anonymization process comprises automatically processing said image-based data at said image-based sensor to extract a skeletal projection or anonymized three-dimensional body representation from said image-based data.

In one embodiment, the predefined data anonymization process comprises automatically processing said image-based data at said image-based sensor to identify a facial border from said image-based data and blurr pixelated content within said facial border.

In one embodiment, the image-based sensor comprises one or more of a colour (RGB) camera, a colour-depth (RGB-D) camera, a depth camera, or a dynamic vision sensor (DVS).

In one embodiment, the respective posture defines any one or more of a body motion, a body posture, an activity level, a predefined action, a predefined behaviour, or a predefined relative body disposition.

In one embodiment, at least one of said anticipated harm scenarios is digitally characterised by a combination of said vital sign, said respective posture, and at least one of a digitally detected presence of a designated object in the vicinity of the individual being monitored, an anomalous presence in the designated environment, or a temperature associated with the designated environment.

In one embodiment, the image-based data is further processed to extract respective digital representations of respective postures of respective individuals within the designated environment, and wherein at least one of said anticipated harm scenarios is digitally characterised by a relative disposition of said respective postures and respective vital signs.

In one embodiment, the network communication interface is communicatively linked to a graphical user interface (GUI) and wherein said digital data processor is operable to output anonymized data representative of the individual in real-time for display via said GUI during monitoring.

In one embodiment, the digital data processor is configured to execute digital instructions to digitally merge said anonymized data to generate a three-dimensional textured scene of the designated environment.

In one embodiment, the image-based sensor comprises a depth-enabled sensor.

In one embodiment, the depth-enabled sensor comprises a time-of-flight infrared sensor or at least two stereoscopic cameras.

In one embodiment, the anticipated harm scenario corresponds to one or more of a self-harm event, a hanging event, a choking event, a suicide attempt, or a fight.

In one embodiment, the designated environment comprises a prison cell.

In accordance with another aspect, there is provided a privacy-maintaining system for automatically monitoring an individual in a designated environment to identify a risk of harm, the system comprising: a sensor array arranged within said designated environment to provide complementary views thereof, said sensor array configured to acquire data of a plurality of data types representative of a current state of the individual, said sensor array including a depth-enabled image-based sensor configured to capture image-depth data of the designated environment; a control interface configured to communicate with said sensor array and a remote device; a digital data processor in communication with said sensor array and said control interface, and configured to execute digital instructions to automatically: via said data of said plurality of data types acquired via said sensor array, extract in real-time a characteristic feature of said current state of the individual; digitally compute using said characteristic feature the risk of harm with respect to an anticipated harm scenario at least partially by implementing a human action recognition process on said characteristic feature; and upon said risk of harm corresponding with said anticipated harm scenario, communicate via said control interface to said remote device an alert corresponding to said anticipated harm scenario.

In one embodiment, the digital data processor is further configured to execute digital instructions to anonymize data of any one or more of said plurality of data types acquired by removing predefined personally identifiable information (PII).

In one embodiment, the digital data processor anonymizes data of any one or more of said plurality of data types acquired by executing a predefined data anonymization process.

In one embodiment, the predefined data anonymization process comprises extracting a skeletal projection from data of any one or more of said plurality of data types acquired.

In one embodiment, the digital data process is configured to implement said human action recognition process at least in part on said skeletal projection extracted so as to detect said characteristic feature.

In one embodiment, the predefined data anonymization process comprises identifying any one or both of a facial border and a bodily border from data of any one or more of said plurality of data types acquired and blurring pixelated content within any one or both of said facial border and said bodily border.

In one embodiment, the predefined data anonymization process comprises extracting optical flow output from data of any one or more of said plurality of data types acquired.

In one embodiment, the digital data processor is configured to implement said human action recognition process at least in part on said optical flow output extracted so as to detect said characteristic feature.

In one embodiment, the digital data processor is configured to receive via the remote device a personally identifiable information (PII) threshold defining at least in part which PII should be actively removed from said data acquired.

In one embodiment, the digital data processor anonymizes data of any one or more of said plurality of data types acquired such that only data required for extracting said characteristic feature is retained.

In one embodiment, the digital data processor anonymizes said data and extracts said characteristic feature concurrently.

In one embodiment, the sensor array comprises one or more of a colour (RGB) camera, a colour-depth (RGB-D) camera, a depth camera, a radar sensor, a thermal sensor, an audio sensor, and a dynamic vision sensor (DVS), and wherein said data comprises one or more of visual images or video, depth-related data, radar data, thermal or infrared (IR) data, audio data, or event data.

In one embodiment, the characteristic feature comprises any one or more of a body motion of the individual, a body posture of the individual, an activity level of the individual, a predefined action of the individual, a predefined behaviour of the individual, a presence of a designated object in the vicinity of the individual, an anomalous presence in the designated environment, or a temperature associated with the designated environment.

In one embodiment, the human action recognition process is configured to distinguish between two or more individuals in the designated environment and is operable to detect recognised motions of distinguished individuals.

In one embodiment, the array comprises a thermal sensor configured to capture thermal data of the designated environment, said thermal data being captured at an inherently anonymized resolution.

In one embodiment, the sensor array comprises a radar sensor configured to capture radar data of the designated environment, said radar data being regarded as inherently anonymized data.

In one embodiment, the sensor array comprises a dynamic vision sensor configured to capture dynamic data of the designated environment, said dynamic data being captured at an inherently anonymized resolution.

In one embodiment, the system further comprises a digital data storage, wherein said digital data processor comprises digital instructions configured to automatically record anonymized data representative of any one or both of said characteristic feature and/or said anticipated harm scenario upon generation of said alert.

In one embodiment, the control interface comprises a graphical user interface (GUI) and wherein said digital data processor is operable to output anonymized data representative of any one or both of said characteristic feature and/or said anticipated harm scenario in real-time for display via said GUI.

In one embodiment, the digital data processor is configured to execute digital instructions to digitally merge data from said plurality of data types to generate a three-dimensional textured scene of the designated environment.

In accordance with one aspect, there is provided a harm prevention monitoring system for automatically monitoring a risk of harm to an individual in a designated environment, the system comprising: a sensor array configured to acquire data of a plurality of data types representative of a current state of the individual; a control interface configured to communicate with the sensor array and a remote device; and a digital data processor in communication with the sensor array and the control interface. The digital data processor is configured to execute digital instructions to automatically: via the data of the plurality of data types acquired via the sensor array, extract in real-time a characteristic feature of the current state of the individual; digitally compute using the characteristic feature the risk of harm to the individual with respect to an anticipated harm scenario; and upon the risk of harm corresponding with the anticipated harm scenario, communicate via the control interface to the remote device an alert corresponding to the anticipated harm scenario.

In one embodiment, the sensor array comprises one or more of a colour (RGB) camera, a colour-depth (RGB-D) camera, a depth camera, a radar sensor, a thermal sensor, an audio sensor, a dynamic vision sensor (DVS) or the like. In some embodiments, the data comprises one or more of visual images or video, depth-related data, radar data, thermal or infrared (IR) data, audio data, event data, or the like. In one specific embodiment, the sensor array comprises at least two colour-depth cameras and at least two thermal or IR sensors, arranged to provide at least two complementary views of the designated environment. In one embodiment, the sensor array further comprises a radar sensor.

In one embodiment, the digital data processor is configured to execute instructions to automatically process sensor data corresponding to different types of the plurality of data types to digitally filter the sensor data to improve computation of the risk of harm.

In one embodiment, the characteristic feature comprises any one or more of a body motion of the individual, a body posture of the individual, an activity level of the individual, a predefined action of the individual, a predefined physiological feature of the individual, a predefined behaviour of the individual, a presence of a designated object in the vicinity of the individual, an anomalous presence in the designated environment, a temperature associated with the designated environment or the like.

In one embodiment, the system further comprises a machine learning-based architecture configured to execute a characteristic recognition process to extract in real-time the characteristic feature.

In one embodiment, the system further comprises a machine learning-based architecture configured to execute a risk recognition process to compute the risk of harm to the individual with respect to the anticipated harm scenario. In one specific embodiment, the risk recognition process computes the risk of harm based on two or more extracted characteristic features. In one embodiment, the two or more extracted characteristic features correspond to a human action and any one of a physiological feature or a thermal feature. In one embodiment, the two or more extracted characteristic features correspond to two or more distinct individuals.

In one embodiment, the anticipated harm scenario corresponds to one or more of a self-harm event, a choking event, an anomalous presence in the designated environment, a vital sign of the individual, a bleeding event of the individual, a seizure of the individual, a fire, a fight or the like.

In one embodiment, the sensor array comprises a depth-enabled image-based camera configured to capture image-depth data of the designated environment, and the characteristic feature is based at least in part on a skeletal projection of the individual. In one embodiment, the sensor array comprises at least one additional depth-enabled image-based camera which is arranged to complement coverage of the designated environment, and the digital data processor comprises digital instructions configured to merge image-depth data from respective depth-enabled image-based cameras to extract the skeletal projection.

In one embodiment, the digital data processor comprises digital instructions configured to implement a human action recognition process on the skeletal projection so as to at least partly compute the risk of harm. In some embodiments, the anticipated harm scenario comprises any one or combination of a self-harm event, a hanging, a choking or a seizure. In some embodiments, the human action recognition process distinguishes between two or more skeletal projections in the designated environment and is operable to detect recognised motions of distinguished skeletal projections so as to at least partly compute the risk of harm. In one embodiment, the anticipated harm scenario comprises fighting.

In one embodiment, the sensor array comprises a thermal or IR sensor configured to capture thermal or IR data of the designated environment, and the characteristic feature comprises a thermal anomaly. In various embodiments, the anticipated harm scenario comprises any one or combination of a fire in the designated environment, an abnormal body temperature of the individual, bleeding on, from or proximate the individual or the like.

In one embodiment, the digital data processor comprises digital instructions configured to implement, upon extraction of the thermal anomaly, a blood recognition process operable to determine whether the thermal anomaly comprises any one or both of a blood intensity or an increasing blood presence. In one embodiment, the blood recognition process implemented by the digital data processor identifies the increasing blood presence at least partly by tracking pixel to pixel correspondence over consecutive thermal or IR images. In one embodiment, the sensor array comprises a depth-enabled imaged-based sensor configured to capture image-depth data of the designated environment, and the blood recognition process further analyzes the image-depth data to identify one or more human activities prior to or during the thermal anomaly.

In one embodiment, the sensor array comprises a radar sensor configured to capture radar data of the designated environment, the characteristic feature comprises a vital sign of the individual, and the risk of harm is at least partly computed by implementing a non-contact vital sign monitoring process. In one embodiment, the vital sign comprises breathing rate and the risk of harm comprises an abnormal breathing rate determined by the digital data processor with respect to chest motion.

In one embodiment, the sensor array comprises an image-based sensor configured to capture image data of the designated environment and a dynamic vision sensor configured to capture dynamic data of the designated environment, and the digital data processor extracts from the image data and the dynamic data the characteristic feature comprising an anomalous human action.

In one embodiment, the sensor array comprises an image-based sensor configured to capture image data of the designated environment, the digital data processor is operable to extract from the image data an optical flow output, and the digital data processor extracts from the optical flow output the characteristic feature comprising an anomalous human action. In one embodiment, the anticipated harm scenario comprises any one of a seizure, fighting or an overdose.

In one embodiment, the designated environment comprises a prison cell.

In one embodiment, the system further comprises a digital data storage and the digital data processor comprises digital instructions configured to automatically record one or more of the plurality of data types as abnormal event data upon generation of the alert. In one embodiment, the abnormal event data is stored on the digital data storage to provide an annotated record of the individual in the designated environment.

In one embodiment, the control interface comprises a graphical user interface (GUI) configured to receive system control parameters. In one embodiment, the digital data processor is operable to display in real-time via the GUI any one or more of the sensed data, the characteristic feature, the anticipated harm scenario or the like.

In accordance with another aspect, there is provided a harm prevention method for automatically monitoring a risk of harm to an individual in a designated environment, the method comprising: via a sensor array, acquiring data of a plurality of data types representative of a current state of the individual; via a digital data processor in communication with the sensor array and a control interface configured to communicate with the sensor array and a remote device, executing digital instructions for automatically: via the data of the plurality of data types acquired via the sensor array, extracting in real-time a characteristic feature of the current state of the individual; digitally computing using the characteristic feature the risk of harm to the individual with respect to an anticipated harm scenario; and upon the risk of harm corresponding with the anticipated harm scenario, communicating via the control interface to the remote device an alert corresponding to the anticipated harm scenario.

In various embodiments, the method may incorporate any one or more of the components described with reference to the system above; may extract any one or more of the characteristic features described above; and furthermore, may identify/detect any one or more of the anticipated harm scenarios described above, without limitation.

In one embodiment, extracting in real-time the characteristic feature comprises executing a characteristic recognition process on a machine learning-based architecture.

In one embodiment, digitally computing the risk of harm comprises executing a risk recognition process on a machine learning-based architecture. In one embodiment, the risk recognition process comprises computing the risk of harm based on two or more extracted characteristic features. In one embodiment, the two or more extracted characteristic features correspond to any one of: a human action and any one of a physiological feature or a thermal feature; or two or more distinct individuals.

In one embodiment, extracting the characteristic feature comprises extracting a skeletal projection of the individual from image-depth data acquired by one or more depth-enabled image-based cameras forming part of the sensor array. In one embodiment, digitally computing comprises implementing a human action recognition process on the skeletal projection so as to at least partly compute the risk of harm. In one embodiment, the human action recognition process further comprises distinguishing between two or more skeletal projections in the designated environment so as to at least partly compute a risk of fighting.

In one embodiment, extracting the characteristic feature comprises extracting a thermal anomaly, of the individual or of the designated environment, from thermal or IR data acquired by one or more thermal or IR sensors forming part of the sensor array.

In one embodiment, digitally computing comprises implementing a blood recognition process on the thermal or IR data so as to determine whether the thermal anomaly comprises any one or both of a blood intensity or an increasing blood presence. In one embodiment, the blood recognition process identifies the increasing blood presence at least partly by tracking pixel to pixel correspondence over consecutive thermal or IR images.

In one embodiment, extracting the characteristic feature comprises extracting a vital sign of the individual from radar data acquired by one or more radar sensors forming part of the sensor array. In one embodiment, the vital sign comprises breathing rate and the risk of harm comprises an abnormal breathing rate computed by the digital data processor with respect to chest motion.

In one embodiment, extracting the characteristic feature comprises extracting optical flow output from image data acquired by one or more image-based sensors forming part of the sensor array; and digitally computing comprises implementing a human action recognition process on the optical flow output so as to at least partly compute the risk of harm.

In one embodiment, the designated environment comprises a prison cell and the sensor array is arranged within the prison cell.

In one embodiment, the method further comprises, via a graphical user interface (GUI) associated with the control interface, displaying in real-time any one or more of the sensed data, the characteristic feature, the anticipated harm scenario or the like.

In one embodiment, the method further comprises, upon the risk of harm corresponding with the anticipated harm scenario, recording one or more of the plurality of data types corresponding to abnormal event data. In one embodiment, the method further comprises storing the abnormal event data in a digital data storage to provide an annotated record of the individual in the designated environment.

In accordance with another aspect, there is provided a harm prevention monitoring system for automatically monitoring an individual located in a designated environment to automatically detect if the individual is being harmed or injured, the system comprising: a depth-enabled camera for acquiring depth-related data of the designated environment; a radar sensor for remotely acquiring radar data from the individual; a control interface for sending output to and receiving instructions from a user; a digital data processor operatively connected to the depth-enabled camera, the radar sensor, and the control interface, and programmed to automatically: simultaneously monitor the individual via the depth-enabled camera and the radar sensor to respectively acquire depth and radar data representative of a current state the individual; extract one or more characteristic features from the depth and radar data in real-time; computationally compare the one or more characteristic features against a preset collection of such features corresponding to a pre-identified harm scenario to automatically identify a possible harm event; and communicate via the control interface a warning as to the possible harm event.

In one embodiment, depth data is automatically processed to filter or discriminate events otherwise identifiable via the radar data to reduce false warnings. In one embodiment, the depth-enabled camera comprises a red, green, blue plus depth (RGB-D) camera.

In one embodiment, the one or more characteristic features include two or more of a predefined body motion, a predefined physiological feature, a presence of a predefined object or the like. In one embodiment, the predefined body motion comprises at least one of a predefined body posture or predefined body gesture.

In one embodiment, the system further comprises at least one of an infrared camera, a near infrared camera, an event camera, an audio sensor, a thermal sensor a dynamic vision sensor or the like.

In one embodiment, the pre-identified harm scenario comprises a self-harm scenario. In one embodiment, the self-harm scenario comprises a suicide attempt. In one embodiment, the designated environment comprises a prison cell. In different embodiments, the pre-identified harm scenario comprises one or more of a potentially fatal intoxication, poisoning or overdose.

In accordance with another aspect, there is provided a harm prevention monitoring system for automatically monitoring an individual in a designated environment to automatically detect if the individual is being harmed or injured, the system comprising: a depth-enabled camera for acquiring depth-related data of the designated environment; a thermal sensor for acquiring thermal data of the designated environment; a digital data processor operatively connected to the depth-enabled camera and the thermal sensor, comprising digital instructions which when implemented: simultaneously acquire the depth-related data and the thermal data to respectively acquire depth and radar data representative of a current state the individual and the designated environment; process the depth-related data and the thermal data to extract one or more characteristic features therefrom in real-time; computationally compare the one or more characteristic features against predefined characteristic feature threshold to automatically identify a possible harm event; and generate a warning as to the possible harm event; and a control interface which is in communication with the digital data processor so as to receive the warning for notification thereof to a user.

In various embodiments, this aspect may incorporate any one or more of the components or features described with reference to the aspect(s) above.

Further aspects provide a harm-prevention system, a harm prevention method, a self-harm prevention system and a self-harm prevention method, all of which are substantially described and illustrated herein.

Notably, any embodiment or aspect described above may be combined with any one or more other embodiments or aspects, thereby providing a further embodiment or aspect of the instant disclosure. Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a schematic diagram of a monitoring system, in accordance with one embodiment;

FIG. 2A is a process flow diagram of an illustrative harm prevention monitoring process, and FIG. 2B is a schematic diagram of an exemplary harm scenario, in accordance with various embodiments;

FIG. 3 is a schematic diagram illustrating an exemplary architecture of an exemplary anomalous event and life sign monitoring system (ALSM), in accordance with one embodiment;

FIG. 4A is a schematic diagram of an exemplary prison cell configuration, and FIG. 4B is a schematic diagram of an exemplary configuration for two prison cells, illustrating various exemplary environments in which an ALSM may be employed, in accordance with some embodiments;

FIG. 5A is a schematic diagram of an exemplary sensor node configuration in the exemplary environment of FIG. 4A, and FIG. 5B is a schematic diagram illustrating an exemplary monitoring zone provided by the configuration of FIG. 5A, in accordance with one embodiment;

FIG. 6 is a screenshot of an exemplary graphical user interface (GUI) associated with an exemplary ALSM, in accordance with one embodiment;

FIG. 7 is a schematic diagram illustrating an exemplary hardware architecture of an exemplary ALSM, in accordance with one embodiment;

FIG. 8 is a schematic diagram illustrating an exemplary software architecture for managing ALSM processes, in accordance with one embodiment;

FIG. 9 is a schematic diagram illustrating a further exemplary ALSM system architecture, in accordance with another embodiment;

FIG. 10 is a screenshot of an exemplary scene depicted using depth data acquired by an exemplary ALSM sensor or sensor array, from which person identification and skeletal data can be extracted for processing by an exemplary human action recognition model;

FIG. 11 is a flow-diagram of an exemplary algorithm evaluation pipeline of a pre-trained channel-wise topology refinement graph convolutional neural network (CTR-GCN) model, which is used as a human action recognition algorithm the embodiment of the ALSM system architecture shown in FIG. 9;

FIG. 12 is a flow-diagram of an exemplary algorithm retraining pipeline of the pre-trained CTR-GCN model implemented after the evaluation pipeline shown in FIG. 11, in accordance with one embodiment;

FIG. 13 is a schematic graphic of one aspect of the human action recognition algorithm, showing unprocessed raw skeletons from three differentially spaced RGB-D sensors, after skeletal tracking in the ALSM system architecture shown in FIG. 9, in accordance with one embodiment;

FIG. 14 is a schematic graphic of another aspect of the human action recognition algorithm, showing a merged skeleton obtained after merging the unprocessed raw skeletons from FIG. 13 within the ALSM system architecture shown in FIG. 9, in accordance with one embodiment;

FIG. 15 is a flow-diagram of an exemplary non-contact vital sign monitoring algorithm, which is specifically designed for monitoring breathing rate in the presence of motion, based on data acquired by sensors of the ALSM system architecture of FIG. 9, in accordance with one embodiment;

FIG. 16 is a flow-diagram of an exemplary thermal image analysis algorithm, which is specifically designed for monitoring for bleeding or the presence of blood, using a bleeding detection framework which forms part of the ALSM system architecture of FIG. 9, in accordance with one embodiment;

FIG. 17 is a screenshot of an exemplary frame from a thermal camera, which forms part of the ALSM system architecture of FIG. 9, on which the thermal image analysis algorithm of FIG. 16 is carried out, wherein the algorithm identifies that the bonding box meets certain conditions for dispatching an alarm indicative of possible self-harm, in accordance with one embodiment;

FIG. 18 is an exemplary thermal image from the thermal camera forming part of the ALSM system architecture of FIG. 9, illustrating that eye detection is computationally prohibitive and facial averages of temperature are less computationally intense to obtain, in accordance with one embodiment;

FIG. 19 is a flow-diagram illustrating an exemplary operation of a graphical user interface (GUI) forming part of the ALSM system architecture of FIG. 9, in accordance with one embodiment;

FIG. 20 is a screenshot of an exemplary GUI illustrating an exemplary normal operation screen, in accordance with one embodiment;

FIG. 21 is a screenshot of an exemplary normal operation screen of the exemplary GUI of the ALSM system, showing real-time 3D skeletal data of an inmate in a cell, in accordance with one embodiment;

FIG. 22 is a screenshot of an exemplary alarm screen of the exemplary GUI of the ALSM system, showing real-time RGB data of an inmate in a cell, in accordance with one embodiment;

FIG. 23 is a screenshot of an exemplary archive screen of the exemplary GUI of the ALSM system, showing available archived data for a particular cell, in accordance with one embodiment;

FIG. 24 is an exemplary side-by-side comparison of a thermal image on the left and an optical flow output on the right, both obtained during a simulated seizure, using another embodiment of an ALSM system, in accordance with one embodiment;

FIG. 25 is an exemplary side-by-side comparison of an infrared (IR) image on the left and an optical flow output on the right, both obtained during a simulated seizure, using another embodiment of an ALSM system, in accordance with one embodiment;

FIG. 26 is a screenshots of exemplary dynamic vision sensor (DVS) data obtained during a simulated seizure, using another embodiment of an ALSM system, in accordance with one embodiment, for comparison purposes with FIGS. 24 and/or 25;

FIG. 27 is a flow-diagram of another exemplary thermal image analysis algorithm, which is configured for monitoring for bleeding or the presence of blood, using a bleeding detection framework, in accordance with another embodiment;

FIG. 28 is a processed thermal image of a bleeding event, illustrating an exemplary coarse segmentation result of an exemplary thermal image analysis algorithm, in accordance with another embodiment;

FIG. 29 is a processed thermal image of the bleeding event shown in FIG. 28, illustrating an exemplary fine segmentation result of the exemplary thermal image analysis algorithm, in accordance with another embodiment;

FIG. 30 is a flow-diagram of an exemplary motion detection framework, which is configured for monitoring a breathing rate of an individual in a target area, in accordance with another embodiment;

FIG. 31 is a schematic diagram of exemplary four breathing rate outputs over time from four separately arranged radar sensors forming part of the motion detection framework of FIG. 30, illustrating the comparability between breathing rate data sensed remotely via radar and sensed in contact with the individual via a Hexoskin™;

FIG. 32 is a screenshot of an exemplary GUI, showing real-time skeleton and radar data obtained via the motion detection framework of FIG. 30, illustrating determination of metrics including human presence, respiration, heartrate and movement;

FIG. 33 is a flow-diagram of an exemplary motion detection framework which incorporates DVS data to monitor for a seizure or fighting in a target area, in accordance with one embodiment;

FIG. 34 is a screenshot of an exemplary backend of an exemplary ALSM system, showing real-time infrared, depth and colour data obtained via three RGB-D cameras arranged similar to as shown in FIG. 5A, illustrating the synchronised views of the ALSM system, parameter configuration possible and annotation prior to storage, in accordance with one embodiment;

FIG. 35 is a flow-diagram of exemplary methodology employed in an exemplary privacy-maintaining system for automatically monitoring an individual in a designated environment to identify a risk of harm, in accordance with one embodiment; and

FIG. 36 is a schematic diagram of another exemplary privacy-maintaining system for automatically monitoring an individual in a designated environment to identify a risk of harm, in accordance with another embodiment.

Elements in the several figures are illustrated for simplicity and clarity and have not necessarily been drawn to scale. For example, the dimensions of some of the elements in the figures may be emphasised relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

In this specification, the term “anticipated harm scenario” is generally used to encompass any or all use cases (events/risks/scenarios) disclosed herein and otherwise envisaged by the inventor. It is to be appreciated, however, that the term “anticipated harm scenario” may refer to a scenario that is predicted to commence, has already commenced or has already ended. Accordingly, the term “anticipated harm scenario” is not to be construed as limited to being anticipatory or predictive only.

In this specification, the term “sensor array” or related terms may refer to two or more sensors of the same type or of different types, as the context will indicate and without limitation. Similarly, the phrase “plurality of data types” may, depending on context, refer to a plurality of the same data types or a plurality of different data types, typically understood with reference to the sensor or camera employed.

In this specification, the terms “camera” and “sensor” may be used interchangeably, as the context will indicate. Furthermore, it is to be appreciated that the term “image” may refer to a single image frame and/or a sequence of image frames (video), as the context will indicate, without limitation.

In this specification, where reference it made to “real-time”, it is to be appreciated that certain events/risks/scenarios may require a certain time window for processing and/or detecting the event from sensed data, and therefore “real-time” may be true real-time or in some contexts, real-time offset by a minimal (sometimes indistinguishable) processing window, without limitation.

Unless defined otherwise, all technical and scientific terms used herein have the same meaning as commonly understood by one of ordinary skill in the art to which this disclosure belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or element(s) as appropriate.

The systems and methods described herein provide, in accordance with different embodiments, different examples in which one or more enclosed individuals may be monitored remotely to detect or identify events or scenarios that may cause a reduction of the physical well-being of one or more of these individuals. For example, the system and method described below may provide for multimodal monitoring of prison inmates for signs of violence or self-harm. These and other such applications will be described in further detail below.

In some embodiments, sensor data or data derived from distinct sensors and/or sensor types may be combined (e.g., via sensor fusion) such that the resulting information and/or analysis has less uncertainty than would be characteristic of same when datasets from different sources are considered independently. One advantage arises from the integration of different (modal) data acquired from complimentary sensors and/or cameras having different sensing point of views (POVs). The proposed systems, in some embodiments, alleviate several drawbacks of single-sensor technology by providing complimentary data from multiple sensor types. For example, the use of a depth camera subsystem to extract motion (posture, gesture, action, velocity vector, etc.) of an individual and feed that data into the analysis of simultaneously acquired radar data to better discriminate one or more features in the classification of the radar signal (e.g. non-contact vital signs such as heart rate, breathing rate, etc.).

For example, image-based data, such as depth-enabled image-based data or complementary image-based data from two or more cameras form which three dimensional image-based data can be computed, can be acquired to extract therefrom a digital representation of an individual being monitored within a designated environment (e.g. prison cell). This digital representation can be extracted directly from image-based data without using user-worn beacons or markers, for example, but rather, from direct image processing, as will be further detailed below. This digital representation can be static, in some embodiments, wherein a posture of the individual can be extracted (e.g. represented by a digitally extract disposition of certain of the individual's body parts, limbs joints, etc. relative to one another and/or relative to the designated environment itself. For example, a digitally recognizable posture may include various standing, seating or lying positions, or again a relative positioning of limbs and joints to represent an event like chocking, fighting, fainting or the like. A posture may also, or alternatively include a disposition of the body or parts thereof relative to the environment, for example, whereby a user body may be recognized in a vertical but hanging posture whose feet are disposed at a distance from the ground or any other recognizable object. Likewise, a time-sequence of postures may be extracted to define a gesture or action, or again, more complex motion sequences that may be recognizable as a predetermined event or series of events.

In combination, any such posture data can be assessed with non-contact vital sign data extracted from another sensor, such as a radar sensor, to further and better define, or confirm, a particular anticipated harm scenario, or again, contribute to distinguishing actual harm scenarios from false positives. These and other such considerations will be detailed further below, as can other combinations and/or multimodal data integrations can be considered, as will be further described below.

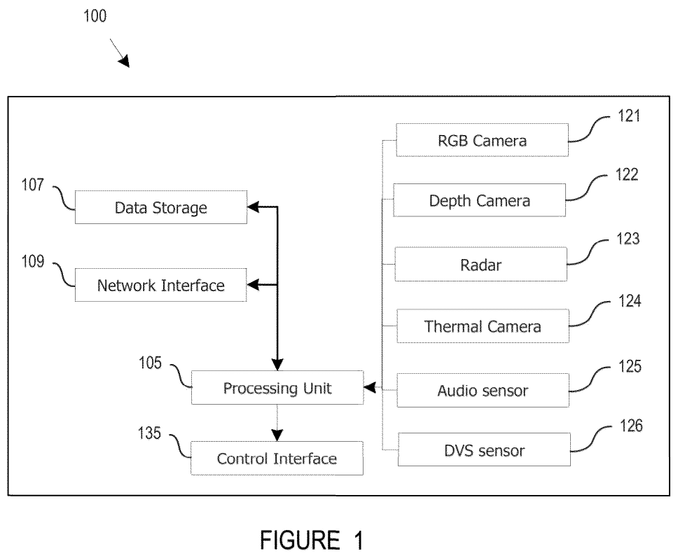

With reference to FIG. 1, and in accordance with one exemplary embodiment, a monitoring system for monitoring the well-being of one or more individuals located in a constrained environment, generally referred to using the numeral 100, will now be described. In some embodiments, system 100 may be a multi-modal monitoring system that integrates data acquired from complementary sensing technologies to monitor one or more individuals. This may include, for example, the monitoring of body movements, gestures, physiological parameters, or the like, to automatically detect or identify problematic events or scenarios (or combinations thereof) that may require intervention from a supervising authority or similar to preserve the well-being of the individual(s) being monitored, such as if an individual is in danger, or is a danger to others.

In some embodiments, system 100 may be used to monitor individuals located within a constrained environment. This may include an indoor environment, such as a prison or prison cell. It may also include a large room featuring multiple individuals interacting with one another, or an outdoor space enclosed by a fence system or the like. Other enclosed or monitored spaces may include, but are not limited to, a hospital or long-term care facility, specialised housing (e.g., for detainees or individuals with severe disabilities), psychiatric wards or care facilities, old age or retirement housing, rehabilitation centres, or the like.

In some embodiments, system 100 may be configured to monitor a combination of human body movements (e.g., motion, gestures, and/or postures), physiological parameters, environmental parameters and/or contextual parameters, to identify an event or scenario that may affect negatively the well-being of one or more individuals being monitored.

For example, system 100, in some embodiments, may be used in a prison or carceral setting, wherein system 100 is used to remotely monitor one or more prisoners for signs of anxious behaviour/nervousness, strangulation (including self-strangulation), self-harm (e.g., self-cutting), a potential overdose, fighting or violent gestures between two or more individuals, and/or the like, and to alert or warn an appropriate (supervising) authority that measures must be taken to prevent/stop such events.

In some embodiments, system 100 may be multi-modal and comprise one or more sensing subsystems configured to acquire different types of sensing data simultaneously. FIG. 1 shows, in accordance with one exemplary embodiment, the system 100 comprising a visible light camera subsystem 121 (e.g., a colour, red-green-blue, or RGB camera subsystem 121), a depth camera subsystem 122 (e.g., including an RGB-D camera subsystem), a radar subsystem 123, a thermal camera subsystem 124, an audio recorder subsystem 125, and/or a dynamic vision sensor (DVS) subsystem 126. In accordance with various embodiments, various subsystems, or combinations thereof, or each subsystem, may thus provide complementary data that, when joined or fused, may provide improved detection of features or events, such as through a reduction in the uncertainty associated with each individual sensing subsystem, for example.

In some embodiments, each subsystem employed may comprise one or more sensors/cameras and/or emitters/receivers of a given type, each of which may be placed in a designated configuration in accordance with and/or adapted for the environment and/or application of interest.

In some embodiments, RGB camera subsystem 121 may comprise one or more digital video cameras, and be operable to detect, via applicable computational vision methods or similar, the presence and/or identity of an individual, including an identifying or characteristic feature (e.g., face or unique body marking) of this individual. An RGB camera subsystem 121 may similarly be configured to detect motion, including gestures and/or postures of an individual. Examples of postures may include standing up, walking, sitting, or lying down. Exemplary gestures may include rapid arm or leg movement (e.g., punching or kicking, or involuntary convulsions), a strangulation attempt, or the like. Other examples of motion may include gait, running or thrusting. In accordance with some embodiments, an RGB camera subsystem 121 may be operable at a variety of settings. One embodiment comprises an RGB camera using a field of view (FoV) of 90°×59° and a resolution of 1920×1080.

In some embodiments, depth camera subsystem 122 may comprise a depth camera operable to provide depth information. For example, depth camera subsystem 122 may comprise one or more RGB-D (i.e., colour-and-depth) cameras/sensors. RGB-D cameras/sensors are depth sensing devices combined with more conventional RGB cameras. The combination of these sensors augments traditional images with depth information to generate 3D textured scenes. In accordance with some embodiments, an RGB-D sensor may comprise an Azure Kinect™, which combines a 1-megapixel infrared (IR) time-of-flight (ToF) depth sensor with a 12-Megapixel RGB camera, although other sensor systems, such as the RealSense™ or a custom-built system may be equally employed, in accordance with other embodiments. In some embodiments, the performance of a particular commercial sensor (e.g., the Azure Kinect™) may be utilised in an RGB-D sensor system, without constraining system functionality to that of the commercial device.

Such sensors may be used to generate complete 3D spatial coordinates of the environment, as well as track and monitor a person(s) within the environment. For example, automatic tracking and extraction of 32 joint coordinates may be achieved with the Azure Kinect™, whereby the ToF imaging system provides high and effective depth image resolution in variable lighting conditions. Further, and in accordance with some embodiments, the Azure Kinect™ depth camera may use a narrow field-of-view (FoV) or wide FOV mode, whereby the narrow mode may offer raw depth data at a FoV of 75°×65°, and a resolution of 640×576, capturing depth data at a range of 0.5 m to 3.86 m. A wide mode may offer raw depth data at a FoV of 120°×120° and a resolution of 1024×1024, capturing depth data at a range of 0.25 m to 2.21 m, in accordance with one embodiment.

In other embodiments, a combination of image-based cameras, such as passive infrared cameras, may be used from different physical perspectives to digitally reconstruct a three-dimensional scene and/or digital extract posture data. Indeed, such a configuration would avoid the continuous pulsed IR emissions required for ToF embodiments, and instead work on triangulated image processing from ambient IR emissions/reflections.

Returning again to FIG. 1, each RGB-D camera provides colour information, as well as the estimated depth for each pixel. As noted, depth camera subsystem 122 may be operable to also extract from the RGB-D pixel data, the presence and/or identity of an individual, including identifying characteristic features (e.g., face or unique body marking) of this individual. The subsystem 122 may additionally or alternatively be configured to detect human motion, such as gestures and/or postures of this individual (e.g., punching, kicking or convulsions). For example, RGB-D data may be used to extract a skeleton model which may be used to compute, for example, posture or gestures. While reference is made to a skeleton model in some of the following examples, it will be appreciated that different digital human representations may be characterized to recognize body postures, gestures, actions and events that are not explicitly limited to an actual skeletal representation, but that may otherwise take different forms such as humanoid volumes or mesh outlines, that may equally be generally characterized as a skeleton or like projection. Accordingly, while the term “skeleton” or “skeletal” is used generally in most descriptive embodiments, the person of ordinary skill in the art will readily recognize that such other forms of digital physiological representations may also be considered within the general context and definition of these illustrative embodiments in ultimately resulting in extracting a digital representation of a posture, gesture or action of the individual being monitored.

In some embodiments, both subsystem 121 and 122 may be operable to detect small objects, including objects being held by an individual, carried on the individual, or generally in the vicinity of, or in contact with, the individual's body. For instance, one or more of the subsystem 121 and the subsystem 122 may be configured to detect the presence of a knife, a gun, rope, or the like, that is disposed nearby the individual.

In some embodiments, a radar subsystem 123 may comprise a continuous wave radar system and/or a pulse radar system, alone or in combination. These may include, without limitation, Frequency Modulated Continuous Wave (FMCW) radar systems, ultra-wideband (UWB) impulse radar systems, or similar systems. For example, a radar subsystem 123 may comprise a low energy level for short-range, high-bandwidth communications over a large portion of the radio spectrum. Such a radar subsystem 123 may be employed, in accordance with some embodiments, for various applications, including precision locating and tracking, as well as life-sign monitoring.

In one embodiment, an UWB subsystem 123 may comprise a Vayyar™ UWB sensor. The sensor may operate in the frequency band from 62-69 GHz, while providing a range resolution of 2.14 cm and an accuracy resolution of 6.7°. The FoV of an exemplary UWB radar sensor may be approximately 180°×82°. In accordance with other embodiments, other radar systems 123, such as the Texas Instruments mmWave™ series, may be employed.

In some embodiments, radar subsystem 123 may be operable to detect and classify human motion based on, at least in part, a micro-Doppler effect, wherein human motion can cause a frequency shift of a radar echo signal, which produces a corresponding Doppler signature. Some examples of features that may be identified using radar subsystem 123 may include physiological features, such as the breathing rate and/or heart rate, or body movement-related features such as gait or rapid convulsions.

In some embodiments, thermal camera subsystem 124 may comprise one or more infrared or near-infrared cameras, and may be configured to acquire heat maps of a physical location or individual. These may be used to, for instance, help locating one or more individuals (e.g., when the lighting level is low); to monitor changes in body temperature, which may be indicative of, for instance, an individual being hurt or in physical distress (e.g., experiencing a fever, reduced metabolism, anxiety, etc.); to identify the presence of bodily fluids (e.g., blood, urine); to identify the presence of hot objects; characterise blood circulation; or the like.

In accordance with one embodiment, a thermal camera subsystem 124 may comprise a Teledyne-Dalsa Calibir GX™ or like sensor. The sensor may be used to detect the body temperature of at least one individual within in its field of view, and/or may be employed to recognise fire or flames in the scene. In accordance with some embodiments, a thermal sensor and/or thermal camera subsystem 124 may be operable with a variety of lens options providing various FoV options. For example, one embodiment provides a FoV of 77°×55° at a native resolution of 640×480. A thermal sensor 124 may be provided with a high dynamic range, with a temperature range of over 1500° C., but, in accordance with some embodiments, may be calibrated to operate in a smaller temperature range at high accuracy (e.g., +/−1° C., from 25° C. to 48° C.). In accordance with another embodiment, a thermal sensor 124 may comprise a Teledyne-Dalsa MicroCaliber™ series, which, for some applications, may be preferred for its small form factor and low cost.

In some embodiments, audio recording subsystem 125 may comprise one or more digital audio recording devices. In some embodiments, these may be placed or affixed at different locations within, for example, one or more rooms. In some embodiments, one or more digital audio recording devices may additionally, or alternatively, be worn by individuals being monitored. In some embodiments, audio recording subsystem 125 may be operable to record and/or analyse ambient sounds or speech from one or more distinct individual(s), and identify therefrom speech patterns or sounds corresponding to one or more signs of distress. These may include for example, an increase in speech volume or a change in pitch, loud noises indicative of violence, gagging sounds, etc.

In some embodiments, a dynamic vision sensor (DVS) subsystem 126, also referred to herein as an event camera 126, may comprise one or more asynchronous sensors that respond to changes in intensity at pixel locations. DVS cameras, in accordance with some embodiments, may provide an increased sensitivity to motion in the scene or constrained environment, and have a much higher dynamic range than other comparable sensors, allowing for changes in intensity to be detected even in low lighting conditions. Furthermore, and in accordance with some embodiments, a DVS subsystem 126 may mitigate otherwise inherent trade-offs between camera speed and data efficiency, allowing for high-speed movement to be captured with significantly less data cost than alternative traditional sensors.

In accordance with one embodiment, a DVS subsystem 126 may comprise a Prophesee Metavision™ or like sensor, which has a high sensitivity to motion and a high dynamic range for high-performance monitoring of human activity level and detection of events such as fighting, spasms, seizures and bleeding. Accordingly, one embodiment relates to a DVS subsystem operable to acquire greater than 10000 frames per second (fps), while providing a FoV of 70° and a resolution of 640×480.

In some embodiments, system 100 may further comprise a processing unit 105, a data storage unit or internal memory 107, a network interface 109, and a control interface 135.

In one embodiment, the processing unit 105 is communicatively linked to subsystems 121 to 125, data storage unit 107, network interface 109, and control interface 135. In some embodiments, processing unit 105 may be configured to operate system 100 as a whole, any one or more of subsystems 121 to 125 individually, or a combination thereof; to process data acquired therefrom using various processing techniques and to store and/or retrieve data from storage unit 107; to communicate data via network interface 109; and to receive and/or send information to control interface 135. In some embodiments, the processing unit 105 is additionally or alternatively communicatively linked to DVS subsystem 126 for similar purposes.

In some embodiments, control interface 135 may comprise dedicated software or program(s) operable to be executed on a general computing device (e.g., desktop, laptop, etc.), and/or personal digital device (e.g., smartphone or tablet). In some embodiments, the control interface 135 may comprise, for example, a graphical user interface (GUI) to configure, operate and receive outputs or messages from system 100.

In some embodiments, network interface 109 may be operable to establish a connection to a communications network, such as the internet or a local network. In some embodiments, network interface 109 may be operable to establish a network connection via Wi-Fi, Bluetooth, near-field communication (NFC), Cellular, 2G, 3G, 4G, 5G or a similar framework. In some embodiments, the connection may be made via a connector cable (e.g., a universal serial bus (USB), including microUSB, USB-C, Lightning connector, or the like). In some embodiments, the system 100 may use a network interface 109 to send or receive information (or messages) to one or more operators/users.

In some embodiments, system 100 may store sensing data acquired by subsystems 121 to 125 (an optionally, 126), or any data derived or combined therefrom to data storage unit 107. In some embodiments, data storage unit 107 may be any form of electronic storage, including a disk drive, optical drive, read-only memory, random-access memory, or flash memory, to name a few examples. In some embodiments, the data storage unit 107 may be present onsite, as part of system 100 or separate therefrom, and in other embodiments the data storage unit 107 may be remote from system 100, accessible for example via the cloud. In yet others, data storage unit 107 may be split between locations.

TABLE 1 below lists different exemplary types of measurements that may be acquired by exemplary subsystems, and the type of data inferred from these measurements that may be used for the purpose of, for instance, body movement or physiological parameter monitoring, in accordance with one embodiment.

TABLE 1

| | TYPE OF | | |
|---|---|---|---|
| SUB- | MEASURE- | DATA | SCENARIO |
| SYSTEM | MENT | EXTRACTED | APPLICATION |
| RBG | Colour video | Object | Recognition of specific |
| Camera | | recognition | objects in view (e.g.: |
| | | | rope); conventional |
| | | | pattern recognition |
| Depth | Acquires | Skeletal tracking | Posture (gesture |
| Camera | depth maps | and/or 3D body | classification); |
| | | motion (e.g., fine | Situational awareness; |
| | | chest motion) | Multi-person |
| | | | interaction; |
| | | | Convulsions |
| Radar | Active Doppler | Micromotions | Breathing rate, |
| | Radar Signal | | Heart rate, Gait, |
| | | | Convulsions |
| Thermal | Thermal map | (Substitute for | Hot patch or bodily |
| | | RGB at night) | fluids; body temperature; |
| | | pattern and object | cell temperature; |
| | | recognition | hot objects; blood |
| | | | circulation; dark/dim |
| | | | room imaging |
| | | | (e.g., false colour |
| | | | imaging) |
| Audio | Ambient | Characteristic | Gagging sounds |
| | sounds | sounds; speech | Anomalous sound(s) |

TABLE 2 illustrates different exemplary combinations of camera/sensor data that may be employed to detect different exemplary complex scenarios, in accordance with various embodiments. For each exemplary scenario in TABLE 2, different exemplary types of elements or features that can be characterise are illustrated, in accordance with different embodiments. However, it will be appreciated that such configurations are not to be understood as limiting. For example, a depth sensor may be employed for, for instance, skeletal tracking purposes to complement other metrics in the evaluation of a potential self-harm scenario.

TABLE 2

| SCENARIO | RGB | DEPTH | RADAR | THERMAL | AUDIO |
|---|---|---|---|---|---|
| 1.Anxious | — | Skeletal | Gait | — | — |
| Behaviour | | Tracking | | | |
| 2. Strangulation | Skeletal | — | BR/HR | Blood | — |
| | Tracking; | | (breathing | circulation | |
| | Gestures; | | rate/heart | | |
| | Posture; | | rate) | | |
| | object around | | | | |
| | neck | | | | |
| 3. Self- | Gestures; | — | BR/HR | Bodily fluid | — |
| harm | object in hand | | | flow at body | |
| | | | | temperature | |
| 4. Overdose | Posture (e.g .: | — | BR/HR, | Body | Anomalous |
| | lying down); | | convulsions | temperature | sounds |
| | convulsions | | | | |
| 5. Fighting | Multiple | Multiple | Activity | — | — |
| | occupancy, | occupancy, | level | | |
| | gestures | gestures | | | |

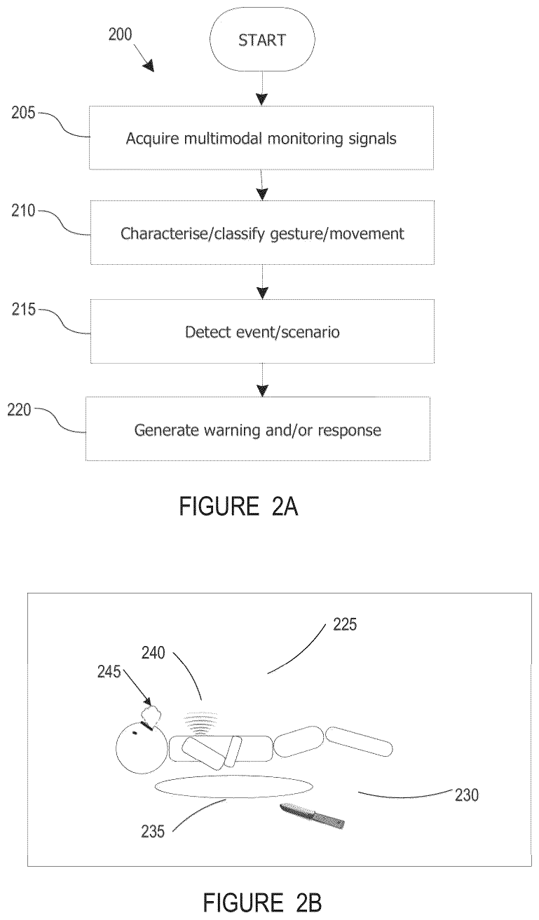

With reference to FIG. 2A, and in accordance with one exemplary embodiment, a process for monitoring the well-being of one or more individuals, generally referred to using the numeral 200, will now be described. In some embodiments, process 200 uses sensing data acquired by system 100, for example by data fusion or similar, to identify scenarios or events where one or more individuals is in danger or a danger to others.

As illustrated in FIG. 2A, at step 205, a monitoring system (e.g., system 100) is activated and actively monitors one or more individuals or locations continuously or at periodic intervals. As discussed above, signals from a multi-modal array of sensors and cameras may be fused and analysed to detect one or more pre-programmed events or scenarios which may require intervention to preserve the well-being of the individual(s) in question.

At step 210, each subsystem may monitor the individual(s) or location(s) simultaneously to detect a multiplicity of features (body motion, physiological signals, object detection or the like), depending on the sensing subsystems employed, as described above. At step 215, these features are analysed to derive therefrom an associated event or risk scenario, which may be achieved, in some embodiments, by comparing data and/or features to pre-existing scenarios configured or defined within the system.

For example, as illustrated schematically in FIG. 2B, an inmate 225 may have attempted suicide while being monitored with an exemplary system (not shown). Features extracted from the RGB-D subsystems may detect that the inmate 225 is lying down, and/or that a knife or sharp object 230 is present near the body 225. A thermal camera(s) may detect hot blood 235 near or coming from the inmate's body 225. One or more additional subsystems, such as a radar subsystem or sound recording subsystem, may detect a reduced heartrate 240 and/or breathing rate 245. Thus, in this example, at step 215 of the process 200, the system may infer from the combination of these data features that the individual 225 has just attempted suicide by cutting himself/herself via a sharp object. Notably, in some embodiments, the system need not identify the event/scenario in such explicit detail, but merely identifying or detecting that such an event/scenario may have or has occurred may be sufficient to solicit an appropriate response. The skilled technician will understand that different complex scenarios may be programmed or configured into system 100, as discussed above.

In some embodiments, for one or both of steps 210 and 215, exemplary system 100 may implement via processing unit 105 one or more machine learning processes, systems, or architectures to perform data analysis, in real-time, or at designated intervals. For example, and in accordance with some embodiments, the data acquired from two or more of subsystems 121 to 125, or changes in such data (e.g., changes representative of motion, a decrease in heart rate, or the like), may be fused or combined to provide more reliable identification of individual features using a machine learning process. In some embodiments, machine learning processes may include supervised learning techniques and/or unsupervised learning techniques. Furthermore, a machine learning process may include, but is not limited to, linear and/or non-linear regression, a decision tree model, principal component analysis, or the like. In accordance with some embodiments, a machine learning process may comprise or relate to one or more of various forms of deep learning. For example, and without limitation, a machine learning process may comprise a deep learning process related to neural networks, such as recurrent neural networks, recursive neural networks, feed-forward neural networks, convolutional neural networks, deep belief networks, or convolutional deep belief networks. Additionally, or alternatively, a machine learning process may relate to the use of multi-layer perceptrons, self-organizing maps, deep Boltzmann machines, stacked de-noising auto-encoders, or the like. In some embodiments, different classification processes may be used to extract features from acquired data, for example a Support Vector Machine (SVM) or similar. As such, system 100, once trained, is designed to operate autonomously or semi-autonomously, with limited or without explicit user intervention.

Once an event/scenario has been identified by system 100 as having had occurred, and/or as is currently ongoing, at step 220, the system 100 may communicate this information to a designated individual(s) and/or device. For example, the system 100 may provide an alert via a GUI interface of control interface 135, or to another computing device via network interface 109. This may be in the form of, for instance, messages sent to one or more individuals, or via activation of one or more alarms.

In some embodiments, messages or alerts may be sent via text, for example via text messages, notifications and/or e-mails or the like. An alert may be provided as a pre-recorded audio message, for example via a phone call or an intercom system, or, in some cases, may include a video component, for example a live video being acquired by RGB camera subsystem 121, or otherwise one or more representative images thereof.

In some embodiments, alerts may include partial or detailed descriptions of the event/scenario identified. Alerts may include additional information, such as the location of the event/scenario, the identity of the individual(s) involved or present in the vicinity, and/or any contextual information that may be helpful.

In some embodiments, system 100 may be configured to provide messages or alerts to one or more pre-authorised individuals. For example, pre-authorised individuals may include wardens, on-duty staff, doctors, attending nurses or the like.

In some embodiments, the warning may be sent, in part, by activating an alarm system or the like.

In some embodiments, system 100 may be operationally connected to one or more automated systems, and be (at least partly or in certain instances) in control thereof or operational to send commands thereto to implement, at least in part, one or more preventive measure. For example, in one embodiment, system 100 may have access to and control of a series of automated doors or the like. Thus, for example, if system 100 detects or identifies fighting or violence between two or more individuals, system 100 may be able to automatically close and lock one or more doors to contain the individuals in question, to prevent involvement of additional participants in the altercation, or the like.

The description above is provided as a general high-level overview of various aspects of exemplary embodiments of the present disclosure. The following description is provided to further elaborate on some of these embodiments, and to provide various additional illustrative examples of a particular class of systems and methods herein contemplated, referred to herein as an Anomalous event and Life Sign Monitoring systems (ALSM) and methods, in accordance with various embodiments.

In accordance with some embodiments, an ALSM comprises an intelligent real-time system equipped with multi-modal and multi-view sensors for the monitoring of life signs and behavioural patterns of one or more individuals, such as prison inmates. Various embodiments of an ALSM provide built-in detection and decision capability for the detection of anomalous events and conditions, such as attempts of self-harm within prison environments. An ALSM may fuse data from a variety of sensors, whereby data may be analysed and/or classified using one or more of various data processing methods or systems, including artificial intelligence (AI) and/or traditional computer vision processes. In accordance with various embodiments, an ALSM may interface with a human operator through a graphical user interface (GUI) to, for instance, display alerts and/or sensor data.

Digital recognition of various application scenarios hereby addressed (e.g., acts of self-harm) are traditionally challenging, wherein the signs and data associated therewith are subtle and diverse in nature. At least in part to address this aspect, various embodiments perform an assessment through the processing of diverse datasets acquired from sensor systems in accordance with one or more of a plurality of sensor units, a plurality of sensor types, a plurality of views, and/or a plurality of classification techniques. Thus, various embodiments relate to the acquisition of a high amount of information to improve or optimise the level at which the anomalous events can be detected and discriminated.

That is, the use of a single sensor and/or sensor type is typically insufficient for effectively addressing all requirements for detection of, for instance, a self-harm event or indeed, can lead to false positives which inevitably reduces the confidence in such systems. Conversely, various embodiments as herein described relate to the integration of disparate or complementary sensors each providing different streams of information, which in turn provides an effective long-term solution for operational use. In accordance with some embodiments, different sensors offer disparate or complementary data, while also providing a degree of redundancy wherein some information or data may be confirmed by more than one sensor. This redundancy may be beneficially leveraged to increase system robustness, in accordance with some embodiments. Notably, the integration of sensor information for these purposes may require complex data processing.

Moreover, various embodiments enable assessment in an operational setting by mitigating challenges associated with occlusion. For example, one embodiment of an ALSM uses three or more viewing angles to allow flexibility in designating a Point-of-View (POV) for viewing a subject or individual from an optimal or improved viewing angle when other POVs are occluded. Furthermore, the ability to merge 3D data from different angles increases the accuracy of fine motion detection, in accordance with some embodiments.

In accordance with some embodiments, an ALSM comprises a multi-sensor platform encompassing ultra-wideband (UWB) radar, color-depth RGB-D sensors, thermal sensors, and Dynamic Vision Sensors (DVS). In accordance with some embodiments, UWB technology is leveraged to track breathing and heart rates, while RGB-D technology is incorporated for 3D imaging and skeletal motion tracking. Thermal imagery provides detection of anomalous heat sources and monitoring of body temperature, while DVS is employed to provide highly sensitive motion detection.

In accordance with some embodiments, multiple classification techniques are leveraged to classify data from each modality. For example, AI classifiers based on neural network-based processes may be used, wherein a customised dataset specific to, for instance, self-harm scenarios, may assist in classification of events. Additionally, or alternatively, an ALSM or process may employ conventional approaches to data analysis to improve system flexibility and operability. For example, more traditional data science approaches may be employed during an early stage of system implementation, such as when a system is deployed in a new setting (or in order to detect a new risk event/scenario), or is used to monitor a risk scenario with respect to a newly monitored individual, or the like.

In accordance with some embodiments, data pre-processing steps may be leveraged to improve performance of the ALSM system, or any one or more subsystems. Such pre-processing steps may be useful, for example, where sensed data is input into a computer vision algorithm or the like, to improve the likelihood of detection of events and/or to reduce the likelihood of false positives.

In accordance with some embodiments, a graphical user interface (GUI) is implemented to control an ALSM to control the system and acquire the output from each subsystem. In some embodiments, the GUI is a high-level GUI. Embodiments may also relate to a decision-tree architecture to determine when an alarm is to be triggered, which may be displayed or otherwise provided by a GUI. It will be appreciated that such an architecture may be modified and adapted to changing requirements in operational use, in accordance with various embodiments.

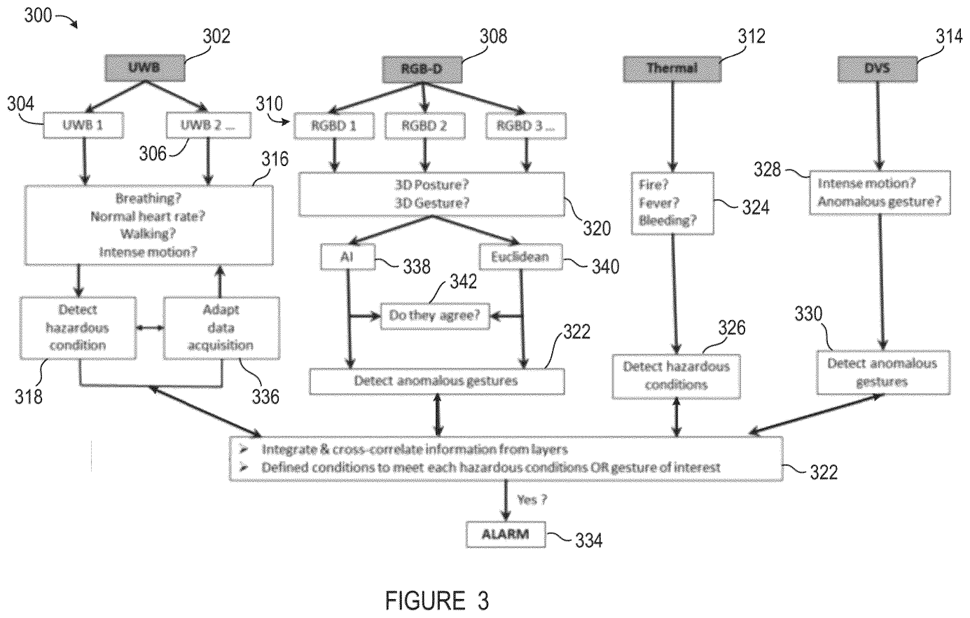

Various embodiments relate to the monitoring of both actions and vital signs of an individual(s) being monitored. For example, FIG. 3 is a schematic diagram of various aspects of exemplary ALSM architecture 300 operable to monitor various aspects of user behaviour and physiology to assess for a potential risk scenario. In this example, the system architecture 300 comprises four sensor types: a UWB sensor array 302, which in turn comprises two UWB sensors 304 and 306, although it will be appreciated that fewer or additional UWB sensors may be similarly be employed within an array 302; a colour-depth camera array 308, which in turn comprises three RGB-D cameras 310, although it will be appreciated that fewer or additional RGB-D sensors may be similarly employed within an array 308; a thermal sensor or imager 312; and a DVS sensor 314. It will be appreciated that the architecture 300 is presented for illustrative purposes, only, and that various other configurations may be similarly employed. For example, other embodiments comprise fewer sensor types (e.g., three of the four sensor types schematically illustrated in FIG. 3), additional sensor types (e.g., five sensor types), and/or that various sensor types may comprise variously sized arrays (e.g., an architecture 300 may comprise a thermal camera array 312 comprising three thermal imagers, while the UWS sensor array 302 comprises a single UWB sensor 304). Moreover, it will be appreciated that various sensors and/or sensor types may be disposed at similar locations, and/or at different locations within an environment. For example, a system architecture may comprise a UWB sensor 304, an RGB-D camera 310, a thermal imager 312, and a DVS system 314 in a common location in a prison cell, while an additional UWB sensor 306 and additional RGB-D sensors 310 are placed at other locations in or viewing the prison cell, so to capture data from other POVs, to mitigate occlusion effects, or the like.

In the exemplary embodiment of FIG. 3, each sensor subsystem captures a unique dataset from which events may be detected (or from which, when considered with other dataset(s), events may be detected). In this case, sensor subsystems 302, 308, 312, and 314 work in parallel to recognise different types of anomalous events, and/or to capture complementary or redundant data with respect to particular or designated event types. For instance, as schematically shown in FIG. 3, the UWS sensor subsystem 302 monitors 316 for, among other aspects and with respect to an individual being monitored, breathing parameters, heart rate, and motion (e.g., if the individual is walking, if they are exhibiting intense motion, or the like) to detect a potentially hazardous condition 318. The colour-depth array 308 may in turn monitor 320 an individual's posture and/or gestures, in up to three dimensions, to detect anomalous gestures 322. Similarly, a thermal imaging subsystem 312 may monitor 324 for hazardous conditions 326, such as anomalous thermal events, a fire, whether an individual has an anomalous temperature (e.g., a fever, a reduction in body temperature, or the like) or an anomalous temperature pattern (e.g., bleeding), while a DVS subsystem 314 may monitor 328 for various motion parameters 330, such as an intense motion and/or anomalous gesture.

In accordance with various embodiments, the ALSM architecture 300 comprises a decision tree layer 332 to, among other aspects, monitor data from each subsystem to support the recognition of different types of events. This layer 332 may engage in, for instance, monitoring data from each subsystem to cross-reference activity or acquired data with user-defined conditions, integrate and/or cross-correlate information between layers, and/or assess acquired data in view of defined hazardous conditions or gestures/parameters of interest. Such assessment may, in the case of recognition of an anomalous event and/or a potential risk scenario, initiate the provision of an alarm 334, for example via a GUI associated with an operator or authority, a siren, the execution of an action (e.g., locking automatic doors), or the like. In some embodiments, the decision tree layer 332 may initiate the alarm 334 when a threshold risk assessment is reached based on the data from each subsystem.

While each sensor subsystem of the architecture 300 may operate independently, various embodiments comprise a data fusion step to augment data collected from individual sensors to improve performance and/or add redundancy, where applicable. This aspect may be performed at the decision layer 332, and may, in some embodiments, include a feedback loop. That is, in accordance with some embodiments, the decision layer 332 may receive data from one sensor which may be used to adjust or calibrate the same sensor, or another sensor(s) interfaced therewith. For example, tracking data from the RGB-D camera system 308 may be used to adjust 336 acquisition or recognition parameters of the UWB radar subsystem 302, for instance to adapt data acquisition 336 for higher-accuracy physiological monitoring. Similarly, various other feedback, calibration, assessment verification, or other comparison processes may be employed in association with any one or more of the various subsystems employed. For example, FIG. 3 schematically shows the evaluation of RGB-D subsystem 308 data by AI 338 and Euclidean 340 processes being compared 342 to determine if an anomalous gesture 322 has been identified. Such assessments may similarly be applied in feedback from the detection layer 332 integrating data streams from other systems to, for instance, improve AI assessment 338, to train an AI 338, or to adjust monitoring parameters when in operation.

In accordance with some embodiments, the architecture 300 is operable to monitor vital signs at a distance or within a range under defined or designated conditions. For example, body temperature may be monitored when there are no significant obstructions or occlusions impairing data capture. Breathing rate may be monitored when the monitored individual is lying still (e.g., sleeping), and when chest displacements caused by breathing are of the same level or greater than those caused by general body movement. Similarly, heart rate may be monitored when the inmate is at rest and not in the vicinity of materials that can interfere with the radar return signal, such as metal. Indeed, the architecture 300 may account for several environmental and/or additive physiological factors which may otherwise impact the ongoing assessment of the individual(s).

The architecture 300 may additionally or alternatively be operable to, in accordance with some embodiments, recognise a variety of pre-defined actions of interest. For example, in one embodiment, the architecture 300 may acquire skeletal tracking data to extract body posture. Body position may then be classified using one or more processes to detect and identify gestures. Similarly, an activity may then be recognised upon the detection of a series of gestures. In accordance with one embodiment, the action of an individual placing their hands at or close to their neck for an extended period of time may be indicative of anomalous activity that could lead to self-harm. In accordance with some embodiments, a set or sets of such rules for each anomalous action may be defined during the development and annotation of a recorded dataset. If the architecture 300 detects a series of gestures that meets such rules, an alarm is generated 334 (e.g., to an operator).

In accordance with various embodiments, various use cases, settings, or applications may warrant respective system conditions and/or requirements. However, in the exemplary scenario of monitoring for a potential self-harm event of an incarcerated individual, various general aspects or system specifications of an architecture 300 or ALSM may apply. For example, an ALSM may be configured to identify, locate and track at least one person within a designated target area of a cell. It may further track joint angles and identify the posture of at least one person in the target area of the cell. Similarly, it may recognise body postures defined as being “of interest” based on skeletal tracking data, and/or a series of body postures leading to actions that have been pre-defined by system users as being “of interest”. These actions of interest may serve as a basis for various use case scenarios, non-limiting examples of which are presented in TABLE 3, where upon recognition of defined conditions, the ALSM or architecture 300 may generate and communicate an alarm to an operator.

TABLE 3

| | Number of | |
|---|---|---|
| Use Case | Tracked | |
| Specifications | Individuals | Conditions to be Identified |
| Multiple | >1 | When multiple people are present in the |
| Presence | | operating environment. |
| Choking | 2 | Hand and wrist joints of one person close |
| | | to the neck joint of another person for |
| | | extended period of time. |
| | | The level of activity of both persons |
| | | registers as ″high″. |
| Hanging | 1 | Hand and wrist joints of one person close |
| | | to their own neck for extended period of |
| | | time. |
| | | OR: joints of the feet are above ground |
| | | for an extended period of time AND the |
| | | person is in vertical position (i.e., not |
| | | lying down). |
| Self-Cuts | 1 | Warm fluid (blood) detected in target area. |
| Fighting | >2 | High degree of body motion by all persons |
| | | in the target area. |
| | | The level of activity in the target area |
| | | registers as “high”. |
| Convulsions/ | 1 | Person is lying down. |
| overdose | | Movements are small and the level of |
| | | activity in the target area registers as |
| | | “high”. |
| | | Or, the breathing rate is registered as |
| | | “low”. |
| Breathing | 1 | Track the breathing rate of one person in |
| | | the target area. |
| Body | >0 | Track the body temperature of at least one |
| Temperature | | person in the target area. |
| Fire Detection | N/A | Detect localised elevated thermal events |
| | | indicative of fire or flames in the target |
| | | area. |

In accordance with various embodiments, system configurations and/or specifications of an ASLM or architecture 300 may be implemented in accordance with a designated or expected environmental configuration. That is, in some embodiments, an ASLM may comprise a particular combination of subsystems, wherein elements of each subsystem are disposed within the environment based on, for instance, a room layout and/or the nature of events for which the monitoring system is being used. Similarly, various monitoring routines, processes, risk assessments, and the like, may be based on environmental conditions, dimensions, angles of view, expected behaviours, and the like.

For example, some embodiments relate to the remote and automatic evaluation of a risk of harm befalling prison inmates, whether from an accident, from self-harm, or from another inmate(s). Accordingly, some embodiments relate to the monitoring of an inmate within their cell, which has a particular and expected geometry, dimensions, fixtures (and fixture placement), and the like. Indeed, such environments are often standardised and/or are in compliance with various requirements defined by a relevant authority, and as such, provide relatively predictable environmental conditions, in view of which an ASLM may be optimally or beneficially implemented with respect to sensor placement, sensitivity, and the like. Moreover, such environments are well suited to computational simulation, for instance to establish an ASLM configuration in advance of practical implementation that is best suited to the environment and risks associated therewith, in accordance with some embodiments.

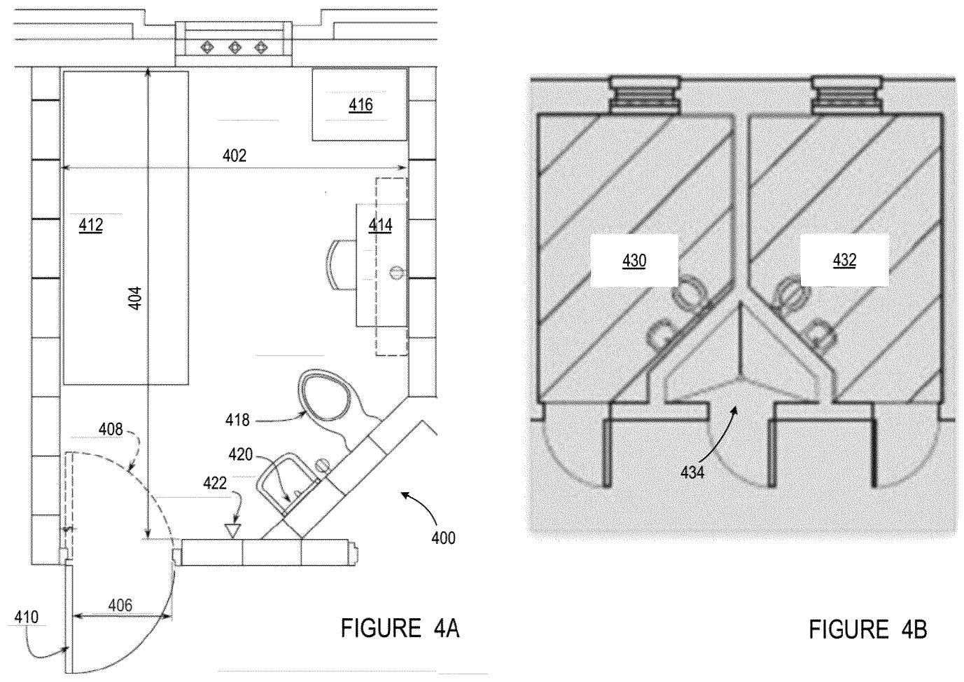

For instance, FIG. 4A shows one exemplary configuration of a prison cell 400. In this example, the configuration of the cell 400 is based on standard requirements defined by a correctional services authority. The floorplan of the cell 400 is thus characterised by standardised dimensions 402 and 404 (e.g., 2.2 m and 3.4 m, respectively) and area (e.g., 7.0 m²), as well as room height (e.g., 2.4 m), which may be considered when placing elements of an ALSM, and/or defining monitoring parameters and/or routines associated therewith. ALSM monitoring parameters and/or configuration may further be designated based on other configurational aspects of the cell 400, such as the width of a doorframe 406, the area of clearance 408 required for the movement of a door 410 that may preclude object placement, or the like, as well as any furniture (e.g., bed 412, desk 414, locker 416) and/or fixtures (e.g., toilet 418, washbasin), or other cell features (e.g., mirror 420, call system 422). FIG. 4B schematically illustrates two cells 430 and 432 similar to cell 400, with maintenance access 434 therebetween. As such cells 430 and 432 are effectively mirror images of each other, and are similarly configured to cell 400, an ALSM for monitoring inmates therein may be similarly configured with mirror-image configurations with predictable degrees of monitoring performance.

FIGS. 4A and 4B present exemplary environmental configurations that may be considered as assumptions, constraints, and/or standards when configuring an ALSM for use within the environment. The standardised configurations of such environments may be further beneficial for determining, in advance of physical implementation, improved or optimal ALSM configurations based using computational modeling or simulations. Whether during practical implementation or digital modeling in advance of implementation, various embodiments of an ALSM may interact within the environment such that various scenarios of interest are effectively captured and accurately assessed in view of anticipated and/or baseline conditions.

In addition to an expected physical environmental configuration, such as the cell parameters described above with respect to FIGS. 4A and 4B, various other environmental aspects may be considered as standard or expected based on expected practical situations, a deviation from which may be considered by the ALSM for assessment as an event. For example, an operational environment for an ALSM within a prison cell may typically be characterised with, nominally, a single occupant within a target area, wherein the detection of an additional occupant may serve as a flag or indication with respect to a potential fighting or choking risk (for example, especially when the additional occupant is identified as another inmate, as opposed to a guard). Similarly, for configuring routines for event recognition, or component placements within an environment, ALSM configuration may further be defined in respect of various expected thermal and/or lighting conditions. For example, an ALSM may be expected to operate in an environment having a temperature of 0° C. and 45° C., while operating at any lighting condition expected for an indoor environment. Moreover, given such conditions, a colour-based camera sensor of an ALSM may only operate optimally during the daytime and under artificial light levels within a given corresponding intensity range, while a DVS component of the ALSM may only operate optimally at low light intensity levels, and light intensity levels higher than those associated with colour cameras. Similarly, an ALSM may operate such that any occlusions arise from small to medium sized objects that, for instance, do not impair the ability of the ALSM to acquire skeletal data. Moreover, the significant metallic content in an environment such as a cell may be largely limited to certain objects, such as a toilet, washbasin, door, bedframe, or desk.

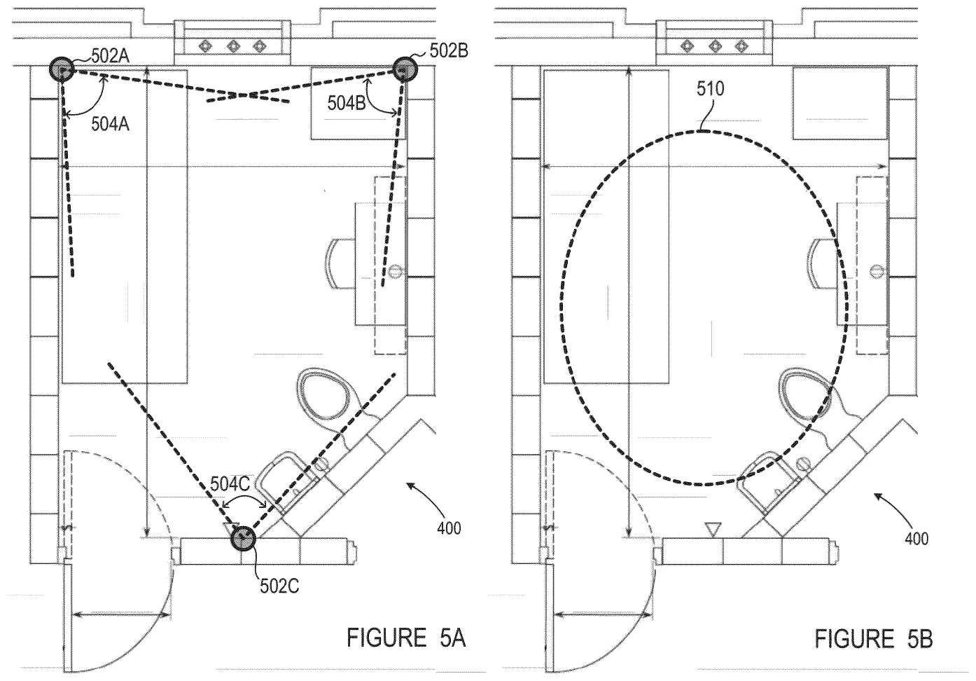

To further illustrate various aspects of an exemplary system design of an ALSM, various use cases will now be described, in accordance with various embodiments. For illustrative purposes, the following examples relate to the implementation of an ALSM within the prison cell 400 described with respect to FIG. 4A. FIG. 5A is a schematic illustrating an exemplary configuration of sensor positions within the exemplary cell environment 400, wherein various sensor and/or subsystems of the exemplary ALSM are disposed at positions 502A, 502B, and 502C, each having respective fields of view/cell coverage angles 504A, 504B, and 504C. In this exemplary configuration, an optimal target area for performing assessments is schematically illustrated by the target area 510 of FIG. 5B. In these examples, and in accordance with some embodiments, the target area 510 may generally be defined as the area covered for which the system may best perform for a given environment or configuration. While there may be a possibility for, for instance, blind spots for which one or more sensor nodes may not be able to monitor, reducing performance for these areas, sensor configuration may be adapted and/or arranged in accordance with a given environment to minimise the number or extent of such areas, in accordance with some embodiments.

In the following description, with respect to each exemplary use case, reference is made to ‘anomalous’ conditions, such as ‘anomalous breathing rate’ or ‘anomalous temperature’. It will be appreciated that such aspects and/or values may be defined by a user through a GUI associated with the ALSM. For example, an operator may set a threshold for anomalous breathing rates to be ‘below 5 breaths per minute’, or they may designate ‘over 20 breathes per minute’, in accordance with some embodiments. Such thresholds may be based on known medical thresholds or indexes, for example. In other embodiments, these thresholds may be predefined based on, for example, training data for a particular use case or environment.

In accordance with one use case, the ALSM is operable to detect when multiple people are present in the operating environment. The system may generate a relevant alarm to the operator if required.

In accordance with one use case, the ALSM is operable to monitor for the action of choking (strangulation) when two people are in the target area. The system may identify a choking event (strangulation event) if the hands and wrists joints of one person are close to the neck joint of the other person for an extended period of time, and the level of activity of both persons registers as ‘high’, whereby the system may generate a relevant alarm for this action to the operator. In accordance with another use case, the ALSM is operable to monitor for the action of self-choking (for example, on food, fluids or an object) when one person is in the target area. The system may identify a self-choking event if the hands and wrists joints of the person are close to their own neck joint for an extended period of time, and the breathing rate of the person changes (optionally other vital signs too, such as heart rate), whereby the system may generate a relevant alarm for this action to the operator.

In accordance with one use case, the ALSM is operable to monitor for the action of ‘hanging’ when one person is in the target area, and when it is detected that the hands and wrist joints of one person are close to their own neck for extended period of time. Alternatively, or additionally, a hanging event may be considered if the joints of the feet are above ground for an extended period of time and the person is in vertical position (i.e., not lying down). The latter may account, for example, where one or more other inmates are involved in the hanging of another. In either case, the system can then generate a relevant alarm for this action to an operator.

In accordance with one use case, the ALSM is operable to monitor for the action of ‘self-cut’ when one person is in the target area. The system may identify conditions for self-cut as the detection of warm fluid (e.g., blood) in the target area, and may generate a relevant alarm for this action to the operator.

In accordance with one use case, the ALSM is operable to monitor for the action of ‘fighting’ when two or more persons are in the target area. The system may identify conditions for fighting as a high degree of body motion by all persons in the target area, and when overall activity in a target area is registered as ‘high’. Upon such identification, the system may generate a relevant alarm for this action to the operator. In some embodiments, coupling of high activity detection with the detection of a sharp object not typically in the FOV, being metallic or otherwise, may aid in identifying fighting and/or a risk of stabbing.

In accordance with one use case, the ALSM is operable to monitor for the action of a ‘convulsion’ or ‘overdose’ when there is a single person identified in the target area. The system may identify such an event when the person is lying down, movements are small, and the level of activity in the target area registers as ‘high’. Alternatively, or additionally, a convulsion or overdose may be automatically identified upon the recognition of ‘low’ breathing and heart rates. In either case, the system may generate a relevant alarm for this action to the operator. These or similar types of events may be applicable not only within the context of long-term incarceration, but also within the context of a temporary hold or imprisonment, for example, where multiple individuals may be held within a common cell or the like pending further processing. Indeed, the systems and methods as described herein may be applied to monitor for harm resulting from intoxication or overconsumption (e.g., alcohol), overdose (e.g., drug-related harm) or the like, whereby such individuals where possibly recently brought in for temporary detention as a result of a crime or public disturbance and still be under the effects of narcotics, alcohol or the like, and whose level of intoxication, for example, may not be immediately addressable by law enforcement personnel. Accordingly, a system as described herein may be deployed to monitor one or more individuals within such a detention environment for any potential signs of significant harm possibly leading to death.

In accordance with one use case, the ALSM is operable to track the breathing rate of one person in the target area. The system will register a breathing rate when the person is at rest and generate a relevant alarm to the operator if an anomalous rate is detected.

In accordance with one use case, the ALSM is operable to track the body temperature of at least one person in the target area and generate a relevant alarm to the operator if an anomalous body temperature or an anomalous body temperature profile is detected.

In accordance with one use case, the ALSM is operable to monitor changes in thermal readings in the target area to detect localised elevated thermal events indicative of fire or flames in the target area, and to accordingly generate a relevant alarm for this action to the operator.

In accordance with one use case, the ALSM is operable to detect one or more harmful objects present in the target area, the presence of a harmful object in the vicinity of an individual, and/or the orientation of a harmful object with respect to an individual(s) (e.g., a knife or sharp unidentified object in the hand of one individual).

Notably, in some use cases (e.g., relating to monitoring for self-harm), the system may be set to a relatively high sensitivity level, whereby more alarms may be generated than strictly necessary, which whilst offering a “safer” system will result in a higher rate of false positives (pertaining to self-harm activities). This may be desirable for high-risk occupants (e.g., known to self-harm). The system may, however, allow for fine tuning of parameters to reach a balance between robustness (reliably generating alarms when needed) and sensitivity (minimizing false alarms).

It will be appreciated that the preceding list of use cases is not exhaustive, and that other types of events or risks may be similarly recognised by an ALSM, in accordance with other embodiments. It is to be further appreciated that, in some embodiments, the coupling of one use case with one or more others herein described may be useful in positively identifying events or risks. This may be particularly so for physiological use cases or data, particularly since physiological parameters are typically variable between normal rates/ranges at resting and abnormal rates/ranges under abnormal conditions. For example, an individual's respiration rate may be predictably variable from a resting respiratory rate to a higher respiration rate in the case of fighting (i.e., onset of stress and/or fight-or-flight response) or a lower respiration rate in the case of a drug overdose.

In accordance with various embodiments, an ALSM may comprise a graphical user interface (GUI) to command, control, archive, communicate, and/or visualise any or all sensor data from one or more of various subsystems of the ALSM. For example, a GUI associated with an ALSM may be operable to display the skeletal data of the observed inmate on request, the measured breathing rate and heart rate of an individual(s) when the observed inmate(s) is (are) at rest, for instance upon request by a user, operator, or administrator. Similarly, the GUI may display the thermal imagery, the level of activity measured in the target area, and an alarm when an observed state meets defined conditions for anomalous events.

The GUI may also allow the user to recall archived data, for instance from a user-defined time range. It may also allow communication between the user and system components through a central server. Such a server may also, in accordance with some embodiments, allow the control software to request and receive data from any or all subsystems, or combinations thereof, process the data, and generate alarms. In accordance with some embodiments, an on-site deployment of an ALSM may include several servers running in parallel. The central server(s) may communicate directly to the data center any to all required information for archiving. Further, a GUI may, at the user's request, access the data center and stored recordings for reporting purposes.

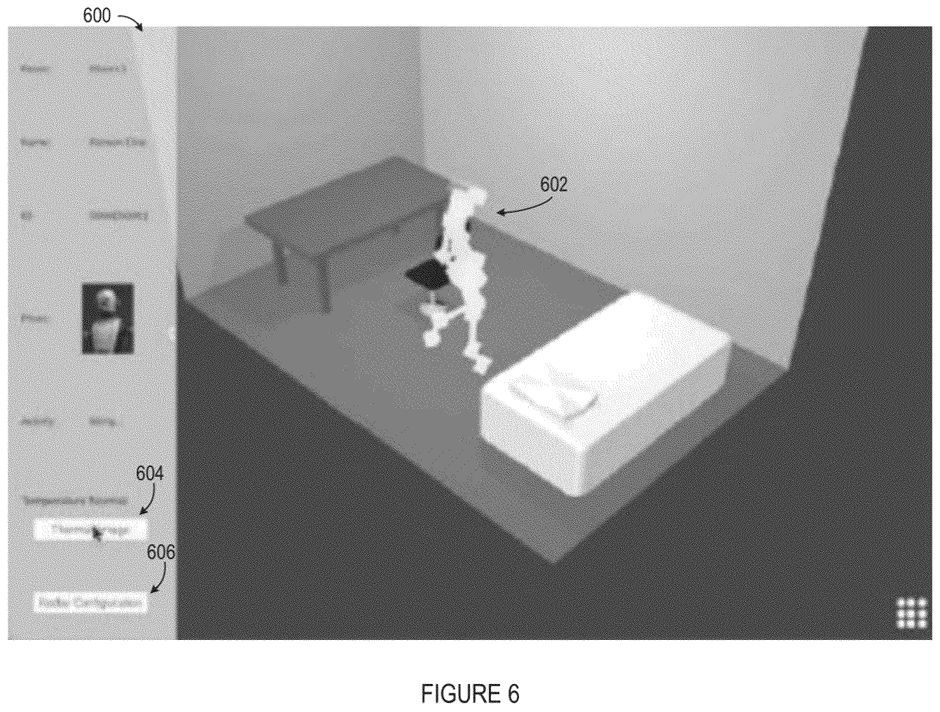

FIG. 6 shows an exemplary GUI 600, in accordance with some embodiments. Such a GUI 600 may comprise, for instance, a digital application executable on a computer or similar machine (e.g., a smartphone, a tablet, a laptop, a desktop, or the like), and may interface in a wired or wireless fashion with various elements (e.g., sensors, servers, or the like) of an ALSM architecture. In this example, the GUI 600 displays real-time skeletal data 602 of an inmate in a prison cell, as detected from acquired RGB-D sensor data from an array. In accordance with one embodiment, the 3D scene of FIG. 6 may comprise a default view of the GUI 600. Alternatively, or in parallel (e.g., via a distinct GUI display panel), the GUI may display a UWB radar point cloud data. In the exemplary GUI 600 of FIG. 6, the inmate representation is projected within a 3D model of the cell room, including other objects (e.g., bed, desk, etc.) therein. In accordance with various embodiments, the model space may be moved, rotated, and/or zoomed to modify the displayed scene, for instance using a mouse, trackpad, or keyboard.

On the left-hand-side of the GUI 600, a pullout information panel displays data of the person in the room, including for example, a detected gesture, temperature, and vital signs. The GUI 600 further comprises user-selectable icons or buttons to display additional or alternative data from the ALSM. For example, the thermal imager icon 604 is user-selectable to generate a pop-up window for displaying thermal images of the target area. Furthermore, the user-selectable radar configuration icon 606, when selected, generates a pop-up window for displaying radar configurational parameters, and may similarly be selected to display radar images, if applicable. Similar pop-up windows may be generated to display and/or control ALSM settings, acquisition parameters, or the like. It will be appreciated that regardless of which sensor type(s) are actively displayed, any or all data being acquired may be continuously monitored in real time, for instance as a background process of the ALSM. Upon recognition of an abnormal or anomalous event, and/or a pre-set or designated risk condition, an alarm is clearly displayed via the GUI 600.

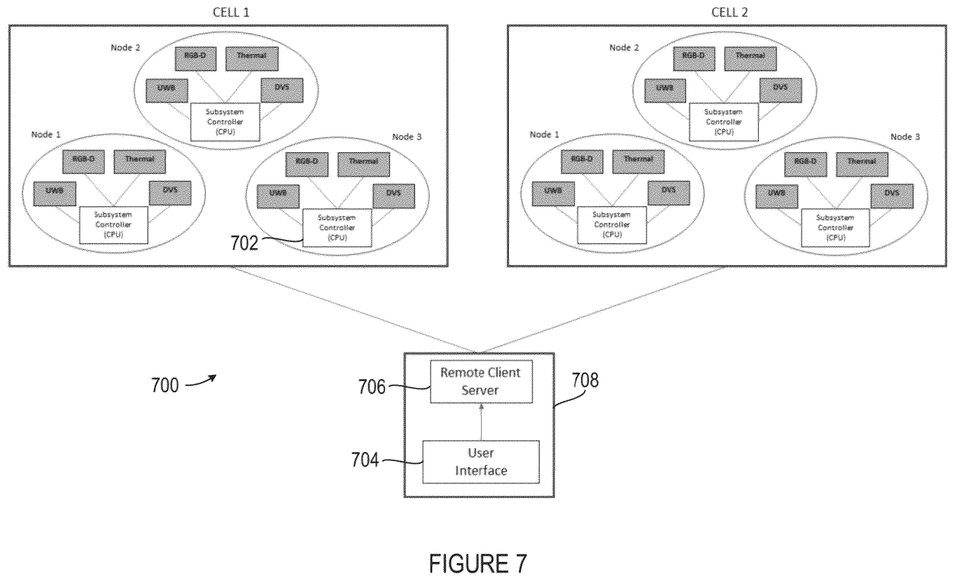

With reference to FIG. 7, an exemplary hardware architecture for an ALSM monitoring two cells (e.g., both cells 430 and 432 of FIG. 4B), generally referred to by the numeral 700, will now be described. In this example, the ALSM architecture 700 is configured, within each cell (labeled as CELL 1 and CELL 2 in FIG. 7), in accordance with three sensor nodes (labelled as Node 1, Node 2, and Node 3 in FIG. 7), wherein each node is disposed within each cell in accordance with a designated configuration to establish a target area for monitoring. For example, Nodes 1, 2, and 3 may be disposed within each cell as schematically depicted in FIG. 5A as sensor placements 502A, 502B, and 502C, respectively, to produce a target area 510, as schematically shown in FIG. 5B. In accordance with some embodiments, nodes may be placed a designated height above the ground, or at ceiling level. It will further be appreciated that particular configuration of nodes within an environment may be dependent on the environment geometry and may be adjusted upon determination of an implementation site, or upon testing the system within a new environment.

In the example of FIG. 7, each node comprises respective sensor arrays comprising an RGB-D camera, a UWB radar sensor, a thermal camera, and a DVS system. Each node has a respective Subsystem Controller CPU 702 to control each sensor type of the node, although it will be appreciated that, in accordance with other embodiments, a plurality of controller units 702 may be associated with a given node, or a given controller 702 unit may interact with sensors from more than one node.

In the two-cell configuration of FIG. 7, a user interface 704 resides on a remote client server 706 accessible from a control post 708 where an operator is stationed. The server 706 is connected to one or more of the nodes inside each cell. While the example of FIG. 7 schematically shows two cells, in some embodiments, one server 706 may handle data management for one, or greater than two cells (e.g., up to 12 cells), and that a particular organisation (e.g., a prison) may have associated therewith any number of remote client servers (e.g., a 100-cell prison may have ten remote client servers 706, each managing data from ten cells). In such broad deployment scenarios, the servers 706 may be placed in separate locations, and provide multiple instances of a user interface 704. The user interface 704, on the other hand, and in accordance with some embodiments, may be deployed on desktops located in control posts 708 or on mobile devices to facilitate monitoring. In other embodiments, for example where only a single cell is monitored by the system, both server 706 and user interface 704 may reside on the same computer in a control post 708. In other embodiments, a user interface 704 may be deployed on multiple control posts 708 of different types, for example may be deployed on a desktop at a guard station and on mobile devices carried by guards on duty.

As noted above, in FIG. 7, each node houses a single instance of several subsystem types (i.e., UWB, RGB-D, thermal, and DVS), as well as a local subsystem controller 702. However, it will be appreciated that each node may have associated therewith any number of similar or alternative subsystem sensors, depending on user needs. Each node may transfer data to the server 706 via various means, non-limiting examples of which may include local area network (LAN), Ethernet Cat 6, wireless connections, or similar, as well as power for the central processing unit (CPU) and the sensors themselves.

In accordance with some embodiments, all sensors in one node may be packaged in a single housing assembly. This may be beneficial for various applications wherein, for example, a particular environment (e.g., a prison cell) has a characteristic configuration, as described above, wherein it is desirable to monitor as large a target area as possible with few nodes and a minimal spatial footprint. This may be further beneficial so as to avoid tampering with sensors and/or nodes of system, particularly when the single housing assembly is robust and tamper resistant.

It will be appreciated that while some embodiments comprise power sources for each or several nodes, others relate to interfacing with existing infrastructure, such as an existing power supply configured in accordance with system requirements. For example, an in accordance with one embodiment, each cell being monitored may be equipped with a 15 A breaker to regulate power to the controllers and sensors of all three nodes.

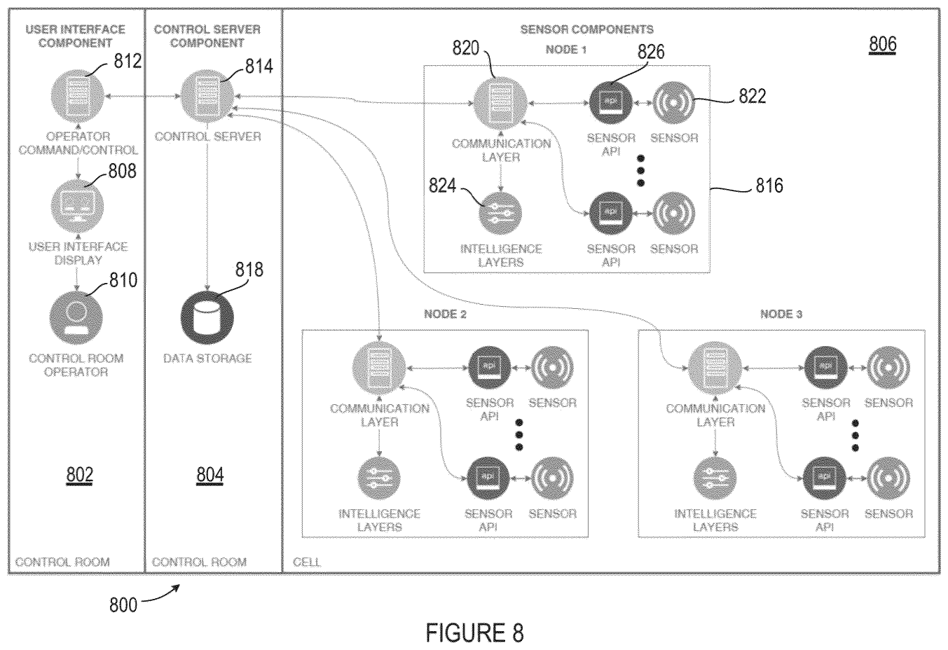

With respect to, for instance, governing the distribution of computational load across sensing nodes and an associated system server(s), and accommodating multiple data streams from multiple forms of sensors, FIG. 8 schematically illustrates an exemplary software architecture 800, in accordance with various embodiments. In this example, the system architecture 800 generally comprises three general components (i.e., a user interface component 802, a control server component 804, and a sensor array component 806), and relates to the management of an ALSM monitoring a single environment, although it will be appreciated that other configurations may be similarly implemented or expanded for assessment of risk scenarios for multiple environments, in accordance with various embodiments.

In the exemplary software architecture 800 of FIG. 8, a user interface component 802 (shown as part of the control room) comprises a system GUI 808 accessible by an operator 810 at a command-and-control module 812. The command-and-control module 812 communicates with a central control server 814 of the control server component 804 (also shown as part of the control room) that is responsible for communication with sensor nodes 816 in the target environment (shown as a cell). The control server component 804, in this embodiment, is also responsible for data management and/or data storage 818 for all acquired sensor data.

In the exemplary embodiment of FIG. 8, sensor components 806 comprise three distinct nodes 816. Each node 816 comprises a communication layer 820 for transferring data to the central control server 814 from one to all sensors 822 in that node 816, which may be interfaced with via a sensor application programming interface (API) 826. The nodes 816 each further comprise an intelligence layer 824 that is operable to execute any applicable processes for the sensors 822 of that node 816. Depending on, for instance, the nature or parameters associated with sensor nodes 816, processes associated therewith may include, for instance, the tracking of individuals and the identification/recognition of physical self-harming gestures, actions or behaviours (GAB), and/or the monitoring physiological vital signs. In accordance with some embodiments, additional or alternative processes, such as those related to data fusion or an increased processing need not be achievable within nodes 816 themselves, may be performed on or at the central control server 804.

It will be appreciated that sensor control and communication may be built in accordance with relevant API/SDKs (software development kits) provided for each sensor. For example, code designed therefor may simplify data acquisition from each sensor, while increased coding resources may be focused on intelligence layer processes, and the control and communication associated with the central control server.

In accordance with various embodiments, an ALSM or associated method as herein described may meet any required certifications required by, for instance, testing protocols associated with impact protection as defined by a relevant authority (e.g., Correctional Services Canada, or the like), and/or electromagnetic compatibility (EMC) standards. It will further be appreciated that, to address privacy considerations, various embodiments may operate in accordance with regulatory frameworks addressing the same. Accordingly, and in accordance with some embodiments, an ALSM or associated method as herein described complies with principles of Privacy-by-Design, including, but not limited to, Privacy-by-Design Assessments (PDA) and Privacy Impact Assessments (PIA).

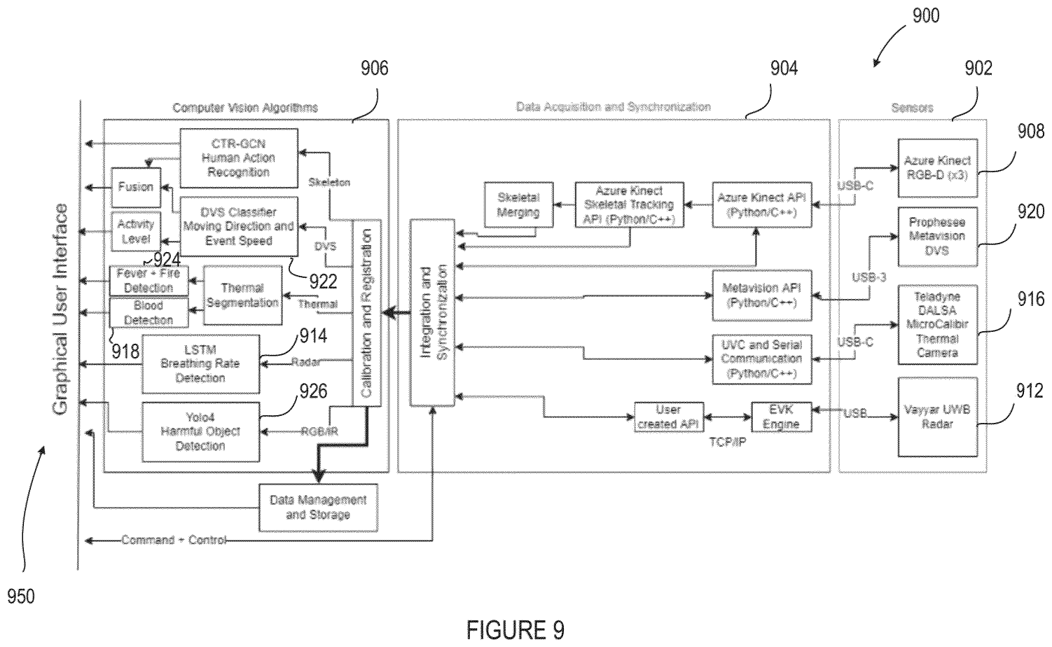

With reference to FIG. 9, and in accordance with a further embodiment, a further ALSM system architecture 900 is shown. In particular, this embodiment exemplifies in more detail, the algorithms or rulesets associated with each sensor subsystem, and thereafter provides examples of algorithm design together with exemplary datasets required. In some instances, comparative analysis and performance evaluation of these exemplary algorithms will be briefly discussed.

In the exemplary ALSM system architecture 900, a set of algorithms that each work on a subset of the acquired sensor data is shown. The sensors 902 are again envisaged installed in a cell, as described above. In this embodiment, each algorithm fulfills one use cases (e.g., as described above). In FIG. 9, the flow of data from the sensor level 902 to each of the algorithms is shown, whereby data is first acquired and synchronized 904 before being parsed through computer vision algorithms 906. Computer vision algorithms 906 in this embodiment include human action recognition, non-contact vital sign monitoring, thermal image analysis for blood detection, confusion reduction using DVS, thermal anomaly detection and harmful object detection, embodiments of each of which will be described further below. Further components of this system architecture 900 will be described with reference to the exemplary algorithms carried out in this embodiment.

In this embodiment, system 900 (and the associated method) relies, at least in part, on AI to detect many of the use cases (actions or risks) described elsewhere herein.

### Human Action Recognition

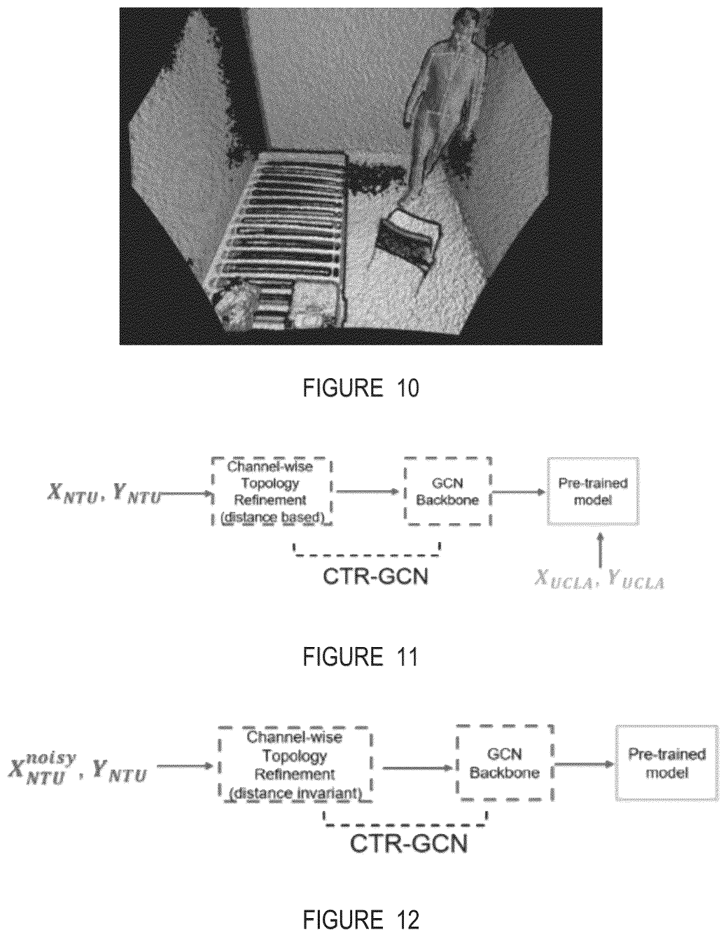

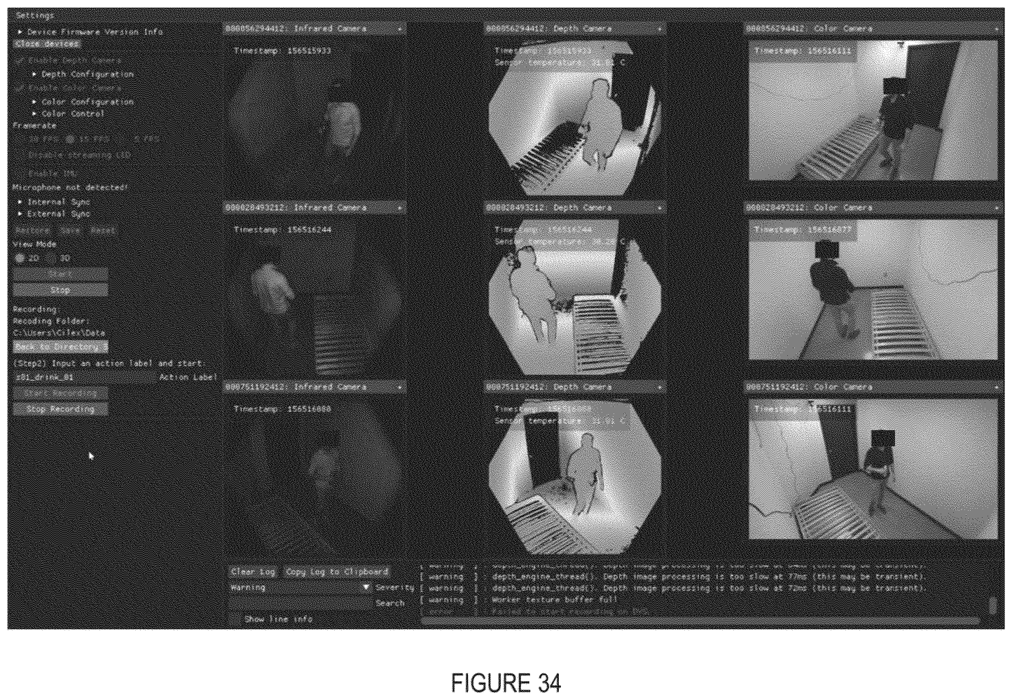

In this embodiment, the sensor level 902 of the system 900 includes an RGB-D subsystem 908, which detects choking, hanging, self-cuts, fighting and convulsions (seizures, due to overdose or otherwise). In this embodiment, the RGB-D subsystem 908 fundamentally obtains IR, depth and colour data (see, for example, FIG. 34 in which this data is obtained from 3 separate locations within the cell, providing different FOVs which are synchronized and simultaneously monitored) on which further processing in carried out. System 900 uses skeletal data to detect these human gestures, actions and/or behaviours, instead of video data as such. As noted elsewhere, such implementation may offer improved computational efficiency and/or may maintain the privacy of monitored individual(s). In this particular embodiment, the system 900 makes use of a graph-based convolutional neural network to process skeletal data, and specifically a channel-wise topology refinement graph convolutional neural network model 910 (CTR-GCN model, interchangeably referred to as a “human action recognition model” or “human action recognition process”). Indeed, use of such a CTR-GCN model 910 in this embodiment allows for the extension of the convolution operation to non-Euclidean data, where in the case of action recognition, skeletons can be represented by the mathematical data structure of graphs, consisting of nodes connected by edges, sometimes with a directionality to the edges. In this embodiment, the network inputs skeleton data as sequences of frames, where each frame contains a fixed number of joint locations in 3D space. Using clips of skeletal sequences, the CTR-GCN model 910 predicts “action classes”. A fixed set of action classes, such as sitting down, clapping of the hands, or touching stomach, are coded into the network using label data prior to training (as noted elsewhere, some action classes may be generic and others may be use case specific). In this embodiment, the training and testing data for the CTR-GCN model 910 is obtained from an RGB-D sensor (similar to sensor 908), where the depth channel is used to predict the joint locations relative to the sensor's position, giving 3D joint locations of one or more individuals in a video sequence, with joint values such as the nose, the right elbow, or the left knee. Indeed, in this embodiment, training datasets consist of sequences of skeletal frames, catalogued for each action class, typically obtained from various camera angles and with different individuals. More specifically, in this embodiment, system 900 makes use of the skeletal joints as extracted by the Microsoft™ Kinect™ sensor's infrared (IR) data (although other joint extraction algorithms may be utilized in other embodiments). FIG. 10 provides a screenshot of an exemplary illustration of a skeletal data frame, wherein a scene in the simulation environment is depicted using the depth data (acquired by, for example, sensor 908 or similar sensor array) from which person identification and skeletal data can be extracted for processing by the CTR-GCN model 910.

In this embodiment, the CTR-GCN model 910 was trained using, for example, the Nanyang Technological University's Red Blue Green and Depth (NTU-RGBD) dataset, which consists of skeletal data from a significant number of actors performing a variety of actions. As known to skilled artisans, an action recognition model (as with any model) trained on a single skeletal dataset will be prone to overfit the same topology distribution of that skeletal dataset during testing. The distribution shift in skeletal data can derive from the variability in the environments with varying architecture and configuration, or the topological discrepancy of skeletal data that is composed with different graph size (varying number of skeleton joints). Accordingly, in this embodiment, a robust model retraining algorithm has been developed for improving the generalization of a pretrained GCN-based skeleton recognition model when deployed on different data distributions.

In particular, to investigate the skeletal distribution shift principal, the model was first tested on another pre-recorded skeletal dataset, Northwestern University and University of California at Los Angeles (NW-UCLA), to identify the potential performance drop across datasets. The algorithm thus presents, in this embodiment, a pre-processing phase on skeletal data, which redefines the label space of two types of skeletal data where the action categories have overlap and redesigns the skeletal graph of NW-UCLA in accordance with the graph topology of NTU-RGBD. The algorithm follows the training implementation of CTR-GCN and trains a normal action recognition model on NTU-RGBD. Afterwards, the pre-trained model was evaluated on the skeletal graph of the NW-UCLA dataset to observe the performance variability. The exemplary algorithm evaluation pipeline of the pre-trained CTR-GCN model 910 is illustrated in FIG. 11. Second, to retrain the GCN model, the Channel-wise Topology Refinement component in the original CTR-GCN framework was re-designed to adapt to the distance-agnostic topological features by considering the structural graph discrepancy across skeletal distributions. Meanwhile, the algorithm 910 is also designed for augmenting the input skeletal data with noise, which builds upon the fundamental network generalization approaches of deep learning theory, aiming to prevent the model 910 from overfitting on a single skeletal data. This exemplary algorithm retraining pipeline for the for CTR-GCN model 910 is illustrated in FIG. 12. In the pipelines shown in FIGS. 11 and 12, X_{NTU} and X_{UCLA} are the two types of skeletal data from two distinct datasets collected by Kinect™ V2, as mentioned above. In FIG. 12, X_{NTU}^{noisy} is the data augmented by Gaussian noise. The dotted box denotes the corresponding component(s) are ready for training. Conversely, the solid frame box denotes that the model 910 is ready for testing or evaluation.

In this embodiment, the retraining and transfer learning results from working with the NTU and UCLA data were then applied to data collected in the simulation environment. During dataset collection, a series of actions relevant to the embodiment were collected alongside several actions previously found in the public datasets (NTU and UCLA). As will be appreciated by skilled artisans, a significant number of actions considered “doing something else” are required in order to develop an algorithm that accurately detects alarm scenarios, while minimizing the number of false positives.

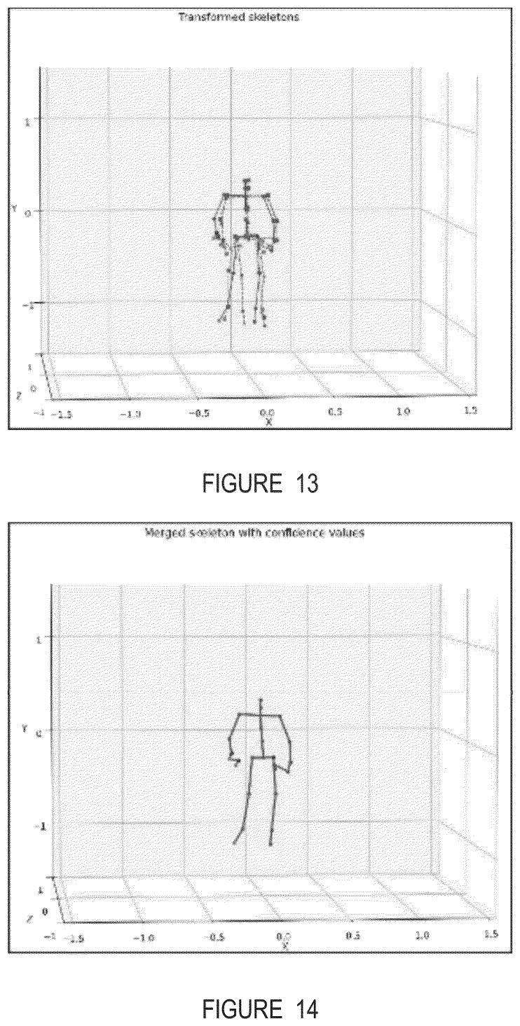

In this embodiment, algorithm 910 includes various pre-processing steps, specifically of the skeletal data, in order to improve algorithmic performance. In particular, due to the location of the cameras in the simulated cell environment, skeleton extraction from the Kinect™ images may not be as precise as many of the public datasets (where the actors are facing a camera at chest height). As such, in this embodiment significant effort may be required to combine skeletal joints with high confidence between views, as well as temporal and spatial smoothing. In this embodiment, therefore, algorithm 910 may include a pre-processing step whereby skeletal data extracted from different cameras/views (e.g., RGB-D sensors 908) is merged into a single skeleton, thereby to improve confidence values. To provide one example, FIG. 13 shows unprocessed raw skeletons from three Kinect™ views and FIG. 14 shows a merged skeleton based thereon, with the confidence values.

In this embodiment, the RGB-D subsystem 908 of system 900 is further operable to detect the presence of multiple people in a target area. This may be beneficial, for example, when monitoring a cell intended to house a single inmate, such that entry of a second inmate (or more) may be indicative of an event/risk (e.g., fighting) which warrants an alarm. In other embodiments, where for example multiple inmates are monitored in a shared room (e.g., dining hall, entertainment room, visitation room or the like), the proximity of persons in the shared room may be monitored in order to detect potential events/risks. For example, if two inmates come into proximity closer than a defined or expected proximity for a certain room, this may generate an alarm to signal potential fighting or other risk activity (particularly when coupled with other data, such as increased motion and/or increased breathing rates or the like).

Notwithstanding the foregoing, in this embodiment of the human action recognition 910, as illustrated in TABLE 4 below, the following subsystems are utilized to assess particular human actions which are considered abnormal events:

TABLE 4

| Event | Subsystem | |
|---|---|---|
| Choking | RGB-D | (e.g., 908) |
| Hanging | RGB-D | (e.g., 908) |
| Self-cuts | RGB-D | (e.g., 908), |
| | Thermal | (e.g., 916) |
| Fighting | RGB-D | (e.g., 908), |
| | DVS | (e.g., 920) |
| Detecting multiple people in cell | RGB-D | (e.g., 908) |
| Abnormal breathing rate at rest | UWB | (e.g., 912) |
| Convulsions/seizure due to | RGB-D | (e.g., 908), |
| overdose | DVS | (e.g., 920) |
| Abnormal body temperature | Thermal | (e.g., 916) |
| Flames or fire in cell | Thermal | (e.g., 916) |

Although TABLE 4 illustrates the different camera/sensor data and combinations thereof that is/are employed in this embodiment, which work cooperatively to provide comprehensive coverage of the target area, it is to be appreciated that other data and combinations thereof may be employed in accordance with various different embodiments. Notably, in some embodiments, the DVS component may be omitted from system 900 entirely, particularly where the coverage afforded by other sensors 902 is sufficient for the use case.

### Non-Contact Vital Sign Monitoring

The system architecture 900 of FIG. 9 further includes a non-contact vital sign monitoring computer vision algorithm, of which one embodiment will now be described. In particular, an embodiment of the non-contact vital sign monitoring computer vision algorithm which is used to determine breathing rate (BR) 914 of an individual(s) will be described, although it is to be appreciated that the same algorithm may be implemented for additional or alternative vital sign use cases, such as for blood pressure and/or heart rate determinations.

In this embodiment, system 900 includes an Infra-Red Ultra Wide Band (IR-UWB) radar 912, utilized to determine the BR of a target subject in the simulated prison environment. As noted elsewhere, the inclusion of this sensor 912 aims to allow for the measurement of the chest displacement due to respiration.

In other embodiments, the system 900 may also include Frequency Modulation Continuous Wave (FMCW) radar, in addition to the IR-UWB radar, since while the FMCW technology relies on the phase information of the received signal from a specific body point, the IR-UWB radar uses the amplitude information to compute the vital signals. For both technologies, built-in Digital Signal Processing (DSP) algorithms on the sensors' APIs can be used in some embodiments to track vital signals, while the ground-truth BR are recorded through a contact vital sign tracking vest. Notably, the inventor(s) have found that that the IR-UWB yields a more accurate estimate of the BR compared to the FMCW radar. However, in both technologies random body movements provoke a deviation between the estimated BR and the ground truth, making the results unreliable when considered in isolation.

Returning to the instant embodiment, it is to be appreciated that body movements cause both frequency and amplitude variation of the received signal, which makes it difficult to retrieve vital signs from the data. Accordingly, in this embodiment, there is provided a supervised machine learning solution which demonstrates potential to effectively separate vital signals from the received response signal in presence of body motion. Indeed, system 900 may employ a smart radar system that detects vital signs in presence of motion, which for example, takes advantage of a pre-trained multilayer perceptron to map the raw data from the received signal of a 2.4 GHz doppler radar sensor to the vital signs.

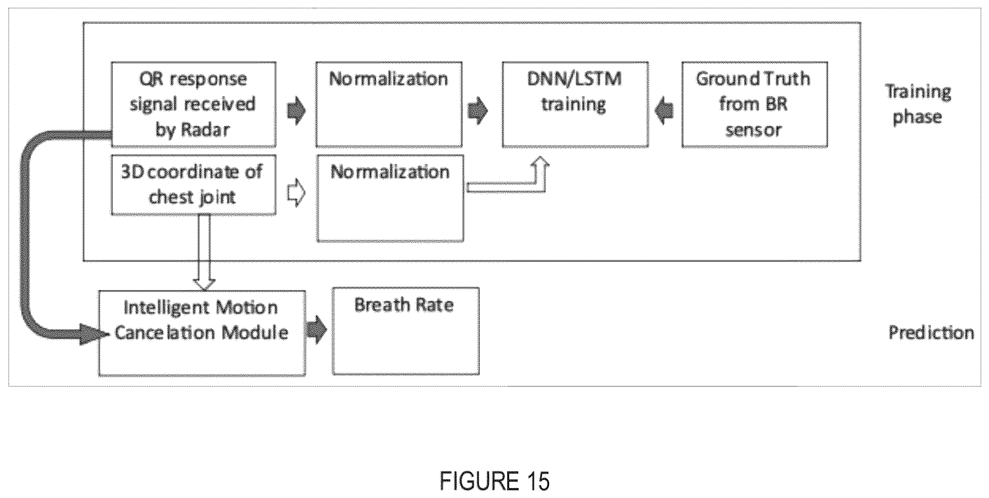

As such, in this embodiment, deep learning solutions for signal parsing (such as DNN, 1D-CNN, LSTM and Gated Recurrent Units (GRU)) are trained to predict the BR in the presence of motion, which forms algorithm 914. For this purpose, a large data set including the pair of transmitted/received IQ signals in presence of motion can be acquired while synchronized ground truth BR are collected using a chest strap sensor. In this particular embodiment, there is proposed a multi-variate deep learning-based solution for elimination of motion artifacts from the received signal of the UWB radar 912 and the displacement of the chest joint extracted from the depth map of the Kinect™ sensors 908. FIG. 15 further illustrates the outline of the structure of a multi-sensor deep learning solution for non-contact monitoring of vital signs (specifically BR, 914) in the presence of motion, based on data acquired by sensors (e.g., 908 and 912) of the ALSM system architecture 900 of FIG. 9. Boxes shown in dotted lines reflect the training phase of this non-contact vital sign monitoring algorithm, and boxes shown in solid lines reflect the prediction phase thereof.

Notably, the BR which is determined from the chest motions detected by the UWB radar (and/or RGB-D sensor 908 data) in this embodiment is compared to predefined (medical) thresholds to ensure that an alarm is generated when the BR is abnormal or otherwise drops below the predefined threshold.

### Thermal Image Analysis for Blood Detection

The system architecture 900 of FIG. 9 further includes a thermal camera 916, and a thermal image computer vision algorithm, of which one embodiment will now be described. In particular, an embodiment of the thermal image computer vision algorithm which is used to detect blood 918 will be described, although it is to be appreciated that the same algorithm may be implemented for additional or alternative thermal use cases, such as for fever and/or fire detection.

In this embodiment, the thermal signature of the target area in the simulation cell, obtained by thermal camera(s) 916, is decomposed into three levels, to distinguish among the background, human body surface and regions on the body with higher temperature including the flow of blood, forehead and between joints where heat is trapped. For this purpose and in this embodiment, a three level Otsu's thresholding algorithm is applied to divide the temperature spectrum of the target area into three levels. Since these threshold values are determined in an offline phase with no object warmer than the individual in the frame, the acquired threshold value remains acceptable for cases where objects with higher temperature are available, however it should be appreciated that a recalculation of the threshold will be required if the environment changes. Once the threshold value is determined, the blood detection algorithm 918 is implemented to detect blood and/or bleeding and to send an alarm.

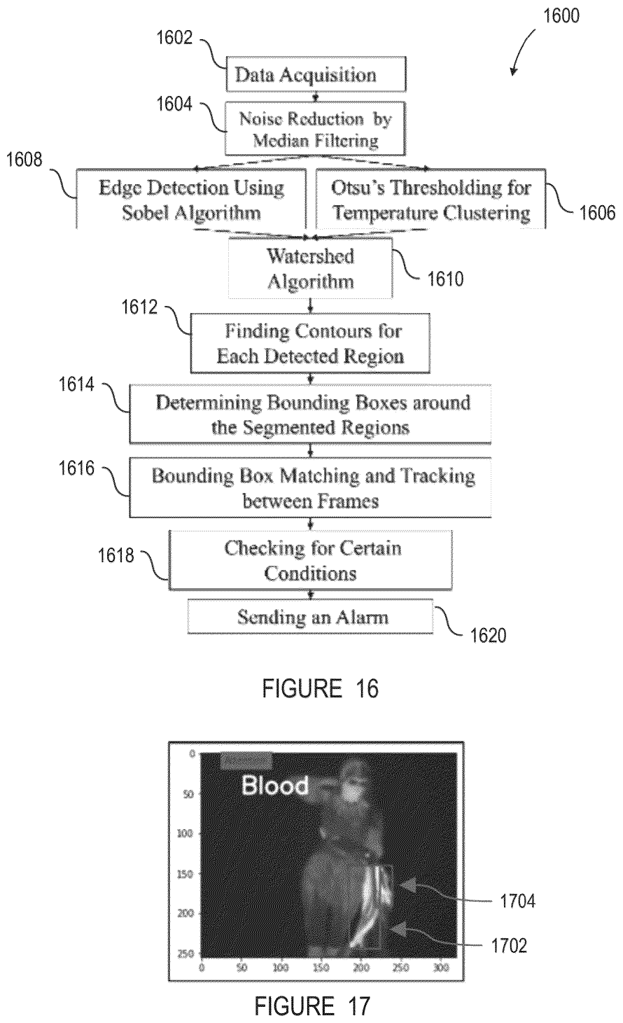

The blood detection algorithm 918 in this embodiment is specifically implemented by a bleeding detection framework 1600, an embodiment of which is shown in FIG. 16. Data is acquired from the target area at 1602, specifically by means of sensor 902 forming part of exemplary system 900. In this embodiment, for any acquired thermal image frame, noise is firstly reduced by median filtering at 1604. The previously mentioned threshold values then cluster the image into three main regions, at 1606, based on the temperature level. The clusters at this stage mainly mark the background, human body, and warmer regions on the body surface; however, such an approach is typically insufficient to perform segmentation because of the noisy nature of thermal images. As such, in this embodiment, at 1608, Sobel edge detection algorithm is then applied to find edges. Thereafter, the main segmentation is performed at 1610, in this embodiment using watershed algorithm with Sobel edge detection as the input image and thermal thresholding map as the markers for the three regions. The watershed algorithm 1610 yields a fine segmentation of the thermal image. Next, at 1612, a contour (sometimes exact) around the regions marked by the highest threshold is found. Once the contours of the regions of interest are detected, a bounding box encountering the region in a rectangular shape is calculated at 1614. At this stage, it is to be appreciated that a series of false detections can be present. As previously noted, these false detections are due to the false reading of blood temperature (37° C.) in the range of forehead on other hot regions on body surface (35-36° C.). At 1616, in order to remove (or at least attempt to remove) false detections, a matching and tracking algorithm is applied between the consecutive frames coming from the thermal camera 916. One embodiment of a pseudo-code of the algorithm for bounding box matching is shown below:

```
for all detected bounding boxes BBⱼⁱ in current frame i
  find the center of the BBⱼⁱ as C_{BB}_{ⱼ}_{ⁱ}
  find the size of the BBⱼⁱ as S_{BB}_{ⱼ}_{ⁱ}
  find the Euclidean distances between all BBⱼⁱ and BB_{j′}ⁱ⁻¹
    if j ≤ j′
      Match the bounding boxes with minimum distance between C_{BB}_{ⱼ}_{ⁱ} and C_{BB}_{_{j′}}_{^{i−1}}
    else
      find the difference between all S_{BB}_{ⱼ}_{ⁱ} and S_{BB}_{ⱼ}_{,}_{^{i−1}}
      Match the bounding boxes with minimum distance between C_{BB}_{ⱼ}_{ⁱ} and C_{BB}_{_{j′}}_{^{i−1}} and
      minimum differences between S_{BB}_{ⱼ}_{ⁱ} and S_{BB}_{_{j′}}_{^{i−1}}
      Mark the remaining BBⱼⁱ as new detection.
```

Once the detected bounding boxes are matched between frames, a series of conditions are checked at 1618 to send an alarm if met at 1620. In this embodiment, these conditions were determined experimentally to distinguish among normal hot regions and hot regions denoting blood flow. Two exemplary conditions include: (1) if a candidate region has an area of smaller than 20 pixels, it is removed as it is likely to indicate small heat trapping between human joints (further, if bleeding is of sufficient seriousness to warrant alarm, then it should produce a larger candidate region in time); and (2) if a candidate region has a bonding box with a height value twice the width, and the center of the associated bounding box is positioned on the lower vertical half of the frame, an “attention” alarm is sent at 1620, meaning that there is a possibility of self-harm (specifically wrist cutting, in this example). The latter condition is determined using the prior knowledge about the position of the thermal camera 916 (an optionally other sensors 902) and about blood flowing in the direction of gravity vector. FIG. 17 shows an example of where the latter condition is met, with exemplary bounding boxes 1702 and 1704 having a height twice the width, being located on a lower vertical half of the frame from thermal camera 916, thereby generating the attention alarm 1706. In this embodiment, if a region is gradually increasing in size within 20 consecutive frames, a more powerful alarm is sent as “blood”, as shown by pop-up notification 1708 in FIG. 17. Accordingly, in this embodiment, algorithm 918 provides an unsupervised blood detection subsystem.

In another embodiment, which is not specifically illustrated, the thermal imaging algorithm for blood detection 918 may be further improved by leveraging IR data acquired by the Kinect™ RGB-D sensors 908. Synchronized and calibrated streams may be captured from the active IR camera 908 emitting 850 nm pulses. Image annotation in the form of bounding boxes around the bleeding region (or similar) may be created and a YOLOv5 object detector (or the like) may be trained on thermal images concatenated with the registered IR image along the third dimension for blood detection. In one embodiment, this object detection algorithm for blood detection may be implemented alongside the unsupervised detection algorithm for comparative analysis and/or potentially improved performance using a fusion of both algorithms. Notably, another embodiment of the bleeding detection framework is described later herein.

### Confusion Reduction Using DVS

The system architecture 900 of FIG. 9 further includes a Dynamic Vision Sensor (DVS) 920, and a DVS computer vision algorithm, of which one embodiment will now be described. In particular, an embodiment of the DVS computer vision algorithm which is used to determine activity level 922 will be described, although it is to be appreciated that the same algorithm may be implemented for additional or alternative motion-based use cases, such as for human activity recognition.

In one embodiment, the DVS algorithm 922 is trained on the following non-limiting exemplary activities: empty room, sitting (no motion), standing (no motion), sleeping, seizure/convulsions, clapping, waving, walking, push ups, fighting, and jumping jacks. In this embodiment, each activity was recorded for approximately 4 seconds. In this embodiment, testing and calculating the level of activity via the DVS 920 based on the mean events per artificial frame for the entire duration of the recording (trimmed from 4 seconds to only include the action itself) renders results as shown in TABLE 5 below. As shown, differences between activities that have a high number of events and those that produce very few events are clear, but differentiation between activities that produce similar numbers of events per frame is required. For example, fighting and working out, or seizure and push-ups. In each case, one of these activities should produce an alarm and the other should not, and therefore in some embodiments, combinatorial sensor data may be preferred.

TABLE 5

| | Activity Level |
|---|---|
| Activity | (1000 events/frame) |
| Empty room | 0.9 |
| Sitting (no motion) | 0.9 |
| Standing (no motion) | 1.0 |
| Sleeping | 0.9 |
| Seizure/convulsions | 4.4 |
| Clapping | 6.4 |
| Waving | 10.2 |
| Walking | 40.8 |
| Push ups | 3.0 |
| Fighting | 148.9 |
| Jumping jacks | 215.8 |

In this embodiment, the DVS algorithm 922 is used for determination of the level of activities of the inmate, as well as detections of activities or situations that are accompanied with fast movements such as seizure, convulsion, or object throwing. More specifically, in this embodiment, the Kinects™ RGB-D sensors 908 are the main sensors for the activity recognition module of the system 900. While the data acquisition sessions are planned in a way to comprehensively cover all possible activities in a cell and enable the core activity recognition network to differentiate among highly similar behaviours, a multisensory fusion system targets the minimization of confusions by calling on outputs from other sensors when the confidence level of the core activity recognition network (CTR-GCN) is low.

One example of these possible confusion cases is the confusion between falling and sitting. Since the major difference between falling and sitting or lying on the ground relates to the speed of the action, the DVS algorithm 922 can be leveraged to distinguish between the two actions, thereby avoiding or aiming to avoid potential false positives of system 900. Indeed, data from DVS 920 and the DVS algorithm 922 may be utilised to supplement or bolster the data from any other sensors 902 in any other algorithms 906 for this or related purposes.

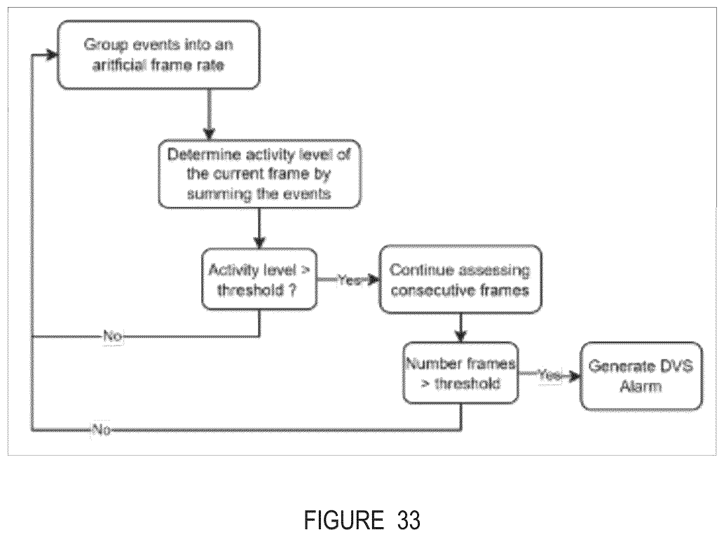

In this particular embodiment, the DVS 920 comprises a Prophesee™ Metavision sensor. Notably, events are generated by the DVS 920 when there is a significant change in intensity at a certain pixel. In addition to identifying the location of the event, the events are also given a polarity, which indicates whether the pixel observed an increase or a decrease in intensity (see, for example, FIG. 26 where the positive polarity is shown in white and negative polarity is shown in blue). In this embodiment, the activity level (which differs from a typical scene, as in a seizure or during fighting) can be calculated by summing the events in the scene over a defined period. Because the DVS 920 does not produce a frame rate like a typical camera, an artificial frame rate is determined and a standard frame rate of 15 fps was used in this embodiment. The artificial frame rate groups events that occur between two timestamps into a standard image with events indicated with or without polarity. With the events grouped into a standard image format, the level of activity for that time period is determined by summing the events in the image. If the activity level is low, the DVS algorithm 922 continues assessing the frames as they are captured. If the level of activity is high, the DVS algorithm 922 assesses the consecutive frames using the same threshold to determine whether the level of activity stays high over time. This step removes artifacts, as well as differentiates the activities of interest (e.g., seizure, fighting) from other motions that may generate a high activity level. One exemplary embodiment of the DVS algorithm 922 which generates or contributes to generating the alarm is shown in FIG. 33.

Use of the DVS 920 in this embodiment captures extremely fast dynamic scenes, with the equivalent of over 10,000 frames per second. The DVS 920 works in extreme lighting conditions due to the large dynamic range; meaning that the sensor 920 can still detect motion in the dark. Lastly, the DVS 920 operates with lower power and lower data transmission rates than a camera operating at the same frequency, because only pixel data where events are detected and need to be transmitted.

As noted, some embodiments of the systems and methods disclosed herein may exclude the DVS 920 and DVS algorithm 922, for example where system performance is achieved using alternative sensors and/or sensor combinations, with appropriate data processing as required. As DVS-excluding embodiments may be useful since, for example, significantly less information is captured by the DVS 920 as compared to other cameras/sensors herein described. Indeed, in static scenes, no information is acquired by the DVS, and it is not as easy to process motion scenes since no pixel intensity information is contained within the DVS data.

### Thermal Anomaly Detection

As noted, the system architecture 900 of FIG. 9 includes a thermal camera 916, and a thermal image computer vision algorithm, of which another two embodiments will now be described. In particular, an embodiment of the thermal image computer vision algorithm which is used to detect other anomalies 924 in the simulated prison environment will be described, which anomalies include fever and/or fire detection.

In this embodiment, thermal anomaly caused by fire will be detected using a simple thresholding algorithm, along with experimentally validated conditions, similar to the blood detection algorithm 918. The threshold used may be acquired from testing a variety of different combustion types (for example, including lighting a fire with match, lighter, burning piece of paper or like, and electrical ignitions). Notably, however, the simple fire detection algorithm is sufficiently robust in this embodiment, at least due to the significantly elevated temperature expected compared to the rest of the cell.

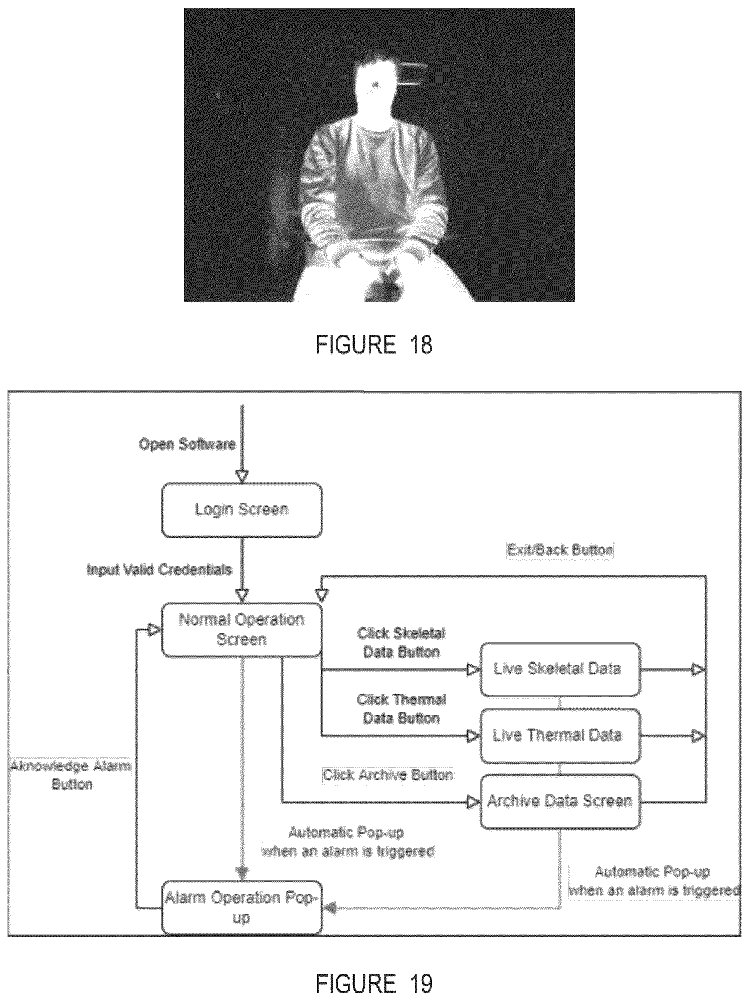

In this embodiment, elevated skin temperature is detected from a distance, without physical contact. Points on the face are detected and the temperature is read using the high-resolution thermal camera 916. It is to be appreciated that the optimal point for temperature sampling using a thermal camera 916 is the inner canthus of the eye. However, in system 900, depending on the angle and distance from the thermal camera 916, this point cannot always be easily identified, and therefore in some embodiments an averaging technique over different parts of the face may more accurately identify the correct temperature. FIG. 18 shows an exemplary thermal image captured with the thermal camera 916 (specifically, a MicroCalibir sensor). From observation from this thermal image, it is evident that even at a short distance from the camera 916, it may not be possible to accurately identify (or at least, without intense computation) the inner canthus of the eye and a full facial averaging technique may be required/useful for detection. Notably, full facial average temperatures may be compared to generic thresholds, or otherwise may be compared to individual-specific thresholds generated after monitoring the individual's thermal profile over time.

### Harmful Object Detection

The system architecture 900 of FIG. 9 further includes a harmful object detection computer vision algorithm 926, of which one embodiment will now be described.

In this embodiment, harmful objects are detected (or aimed to be detected) using IR images. Although harmful object detection on RGB images is workable in different embodiments, this embodiment of the ALSM system 900 includes a harmful object detection algorithm 926 using IR images to detect objects of interest (i.e., harmful objects) without relying on RGB data. Additionally, using IR images gives the benefit of detection in low-light conditions.

In this embodiment, harmful object detection algorithm 926 is based on the You Only Look Once (YOLO) algorithm, which comprises a single-stage object detection framework focused on real-time applications (notably, the usage of other models such as Fast R-CNN or Faster R-CNN are envisaged for other embodiments). To develop this harmful object detection algorithm 926, version YOLOv5 was trained on images for harmful object detection that were taken from the public domain, using pictures of knives from public color and grayscale image datasets. For example, training datasets may include images of individuals holding knives in realistic poses (note that purely classification datasets require additional annotation). More training data may be collected and annotated within the simulated environment, for training or evaluating the harmful object detection algorithm 926.

Notably, in this embodiment, the YOLO family of models may offer increased process speed by reframing the object detection task as a mix of regression and classification, executed in a single stage. The specific model chosen for use in this embodiment is the YOLOv5m (medium) architecture, using a modified Darknet-19 CNN backbone, with 19 convolutional layers, composed of convolutional layers with batch normalization and SiLU activation, as well as convolutional layers augmented with residual connections using bottleneck layers as in the ResNet architecture. In addition, feature maps at strides 8, 16, and 32 are accumulated as in the feature pyramid network structure, where smaller resolution feature maps are used to detect larger objects, and higher resolution feature maps are used to detect smaller objects. A single detection head, consisting of convolutions, is used to predict intersection over union (IoU) values, class-labels, and bounding box regression coordinates for each grid cell of each layer of the feature pyramid network. The ground truth data consists of color images annotated with bounding boxes encapsulating objects using the xywh: x-center, y-center, width, and height format, normalized by the dimensions of the image. Bounding box regression is computed with respect to hand-chosen anchors at each level of the feature pyramid network, where anchors whose respective sizes in relation to ground truth boxes are sufficiently large are paired with a single ground truth box based on the intersection over union criterion. The model then predicts at each grid location, in each feature map of the pyramid, the offsets and scaling factor based on the anchor boxes to match the relevant ground truth box at that location. The CIoU loss is used during training, making use of three criterion: the size of the smallest bounding box encapsulating the predicted and ground truth box, the distance in centers between the boxes, and the difference in aspect ratios of the two boxes.

In this embodiment, data augmentation techniques for YOLOv5 requires an implementation decision differing per application; however may include the use of mosaics, in which 4 images and their respective ground truth boxes are merged in a single training image (with possible resizing); and/or MixUp, in which two ground truth images are merged as the normalized sum of two images, where both sets of ground truth bounding boxes are kept intact during training. In this embodiment, these augmented images are then fed into an additional augmentation module, including standard data augmentation techniques like horizontal/vertical flipping, color jittering, random resize and cropping, perspective/affine warping, brightness/contrast changes, and other techniques.

In this embodiment, training for the detection of harmful objects included collecting sufficient IR images that include both positive and negative samples of objects of interest; annotating the images and retraining the network on the IR images for a harmful object detector using IR images; and performing network optimization after the preliminary results have been determined. In some embodiments, harmful object (e.g., knife) detection alone may be sufficient to trigger an alarm. In other embodiments, for example related to self-harm, harmful object (e.g., knife) detection may be insufficient information to trigger an alarm, hence pose predictions of the knife corresponding to dangerous uses, or combining knife detection with blood detection, may be necessary to obtain a holistic appraisal of the nature of the behaviour being displayed by a subject, and/or to avoid false positives.

In this embodiment, the system architecture 900 includes a graphical user interface (GUI) 950 which, in this embodiment, is used to convey sensed data from sensor 902, raw and/or processed using any one or more of the described algorithms, to an operator and when applicable, conveying an alarm to the operator. FIG. 19 provides one exemplary embodiment of the GUI 950 operation.

The GUI 950 of system 900 in this embodiment comprises a variety of screens displaying different data sensed by sensors 902, and screens are navigated between using the relevant GUI buttons. As shown in FIG. 19, upon opening the GUI 950 software, the operator will be required to log in to ensure only credentialed users can use the system (appreciating that multi-factor authentication steps or the like may be required in different embodiments) before the normal operation screen will be displayed. From this screen, the operator can toggle between various data streams, or combination data streams, most of which provides real time or live data to the operator. Archive data (and specifically, archived alarms) is also available in this embodiment.

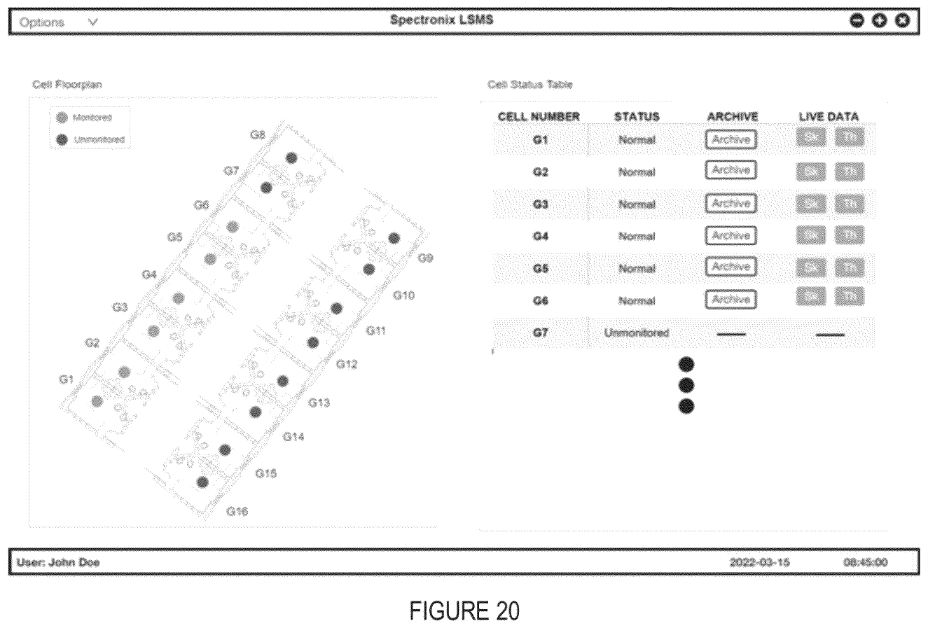

One embodiment of the normal operation screen is shown in FIG. 20. In this embodiment, the normal operation screen shows all cells in the cell block (i.e., G1 to G16), and provides an overview of all monitored cells (i.e., G1 to G6 only) in the “Cell Status Table”. In this embodiment, when a particular cell is selected in the “Cell Status Table”, the GUI 950 will highlight the cell in the cellblock floorplan. In this embodiment and as shown in FIG. 20, buttons for each cell can be clicked by the operator to bring up archived alarms, live 3D skeletal (Sk) data and/or live thermal (Th) data from any one or combination of sensors 902. The cell status will change from “normal” to, for example, “alarm” or “accident”, when an event/risk is detected and the alarm is active. In this embodiment, the “alarm” status automatically changes back to “normal” once the alarm is acknowledged (in other embodiments, further acknowledgments and/or response steps may be required before the status reverts to “normal”, such as attendance by a healthcare practitioner or review by a warden, or the like). This may ensure, for example, that where the real-time data ceases to indicate an event (e.g., self-harm, fighting, fire, etc.), that the operator(s) are still notified of same should a follow-up be prudent. To provide a few examples: if a seizure was detected but the seizure ended, attendance by a medical professional may still be advisable; if a fight was detected but the fight ended, disciplinary actions may still be relevant; if self-harm action was detected but ended, appropriate psychological therapy may be advisable. Based on the foregoing and as described further below, the ALSM is therefore operable to switch between “normal” and “alarm” operational modes or conditions, specifically on a per cell basis (or per designated environment).

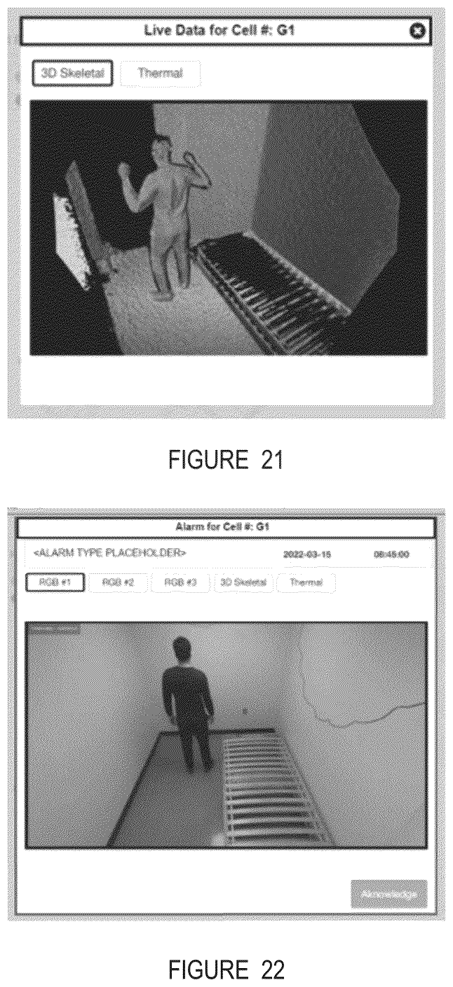

One embodiment of the live data screen is shown in FIG. 21. In this embodiment, the live data screen shows live or real time data from a selected cell (e.g., G1, and the inmate in the cell), specifically when under normal conditions (i.e., no event/risk of self-harm or the like has been identified). As shown, the GUI 950 in this embodiment allows the operator to toggle between 3D skeletal data (Sk) and thermal data (Th) of the same cell, in real time. In this embodiment, system 900 presents only unidentifiable video and data in normal operation mode (3D Sk or Th) which may reduce the operational load on system 900, for example. Notably, in one embodiment, this live stream data may be optionally used by an operator/guard to conduct or otherwise supplement security checks and rounds, ensuring that inmates are present in their cells, appearing to be in good health and/or are not carrying out any potential damage to property.

One embodiment of the alarm screen is shown in FIG. 22. In this embodiment, the alarm screen will automatically pop up when an alarm is triggered for a particular cell(s) and/or inmate(s) by system 900. As shown, the GUI 950 in this embodiment allows the operator to toggle between more data than is typically available in normal conditions, to further supplement review of potential events/risks. In this embodiment, system 900 presents on the alarm screen of FIG. 22 live or real-time RGB data (from all available views, although in some applications 1 or 2 views may suffice), 3D skeletal data and thermal data. In this embodiment, system 900 also determines and displays, based on its application deployment, an alarm type which may be, for example, “self-harm risk”, “bleeding risk”, “hanging risk” or the like (in FIG. 22, only a placeholder is shown). In this particular embodiment, real-time RGB video of the cell is the default view that displays when the alarm screen pops up, as shown in FIG. 22, allowing the operator to quickly obtain an overview of the scenario in the cell. However, in other embodiments, other data may be displayed initially, as suited to the particular application. In other embodiments, the alarm screen (whether displaying RGB data, Sk or Th data) may further include additional annotations (such as bounding boxes etc.) on the image, depending on alarm type and/or configuration of alarms. Such additional annotation may aid in the operator readily identifying, for example, a potentially harmful object (e.g., knife, rope) or scenario in the displayed data. As noted, in this embodiment all alarms must be acknowledged to close alarm screen and return to normal operation. In this embodiment, this is achieved by merely selecting the acknowledge button shown. Acknowledgement of an alarm may prompt system 900 to archive the data associated with the respective alarm, for future access by operators.

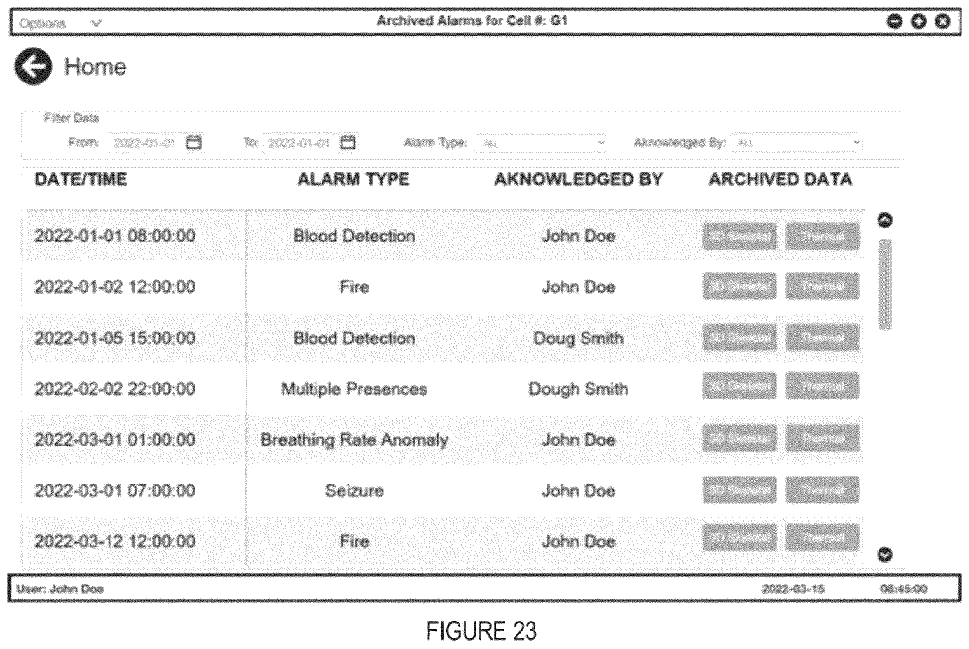

One embodiment of the archive screen is shown in FIG. 23. In this embodiment, the archive screen for a particular cell is accessible via the normal operation screen (i.e., a home screen). Access to archived alarms via system 900 may be useful for several reasons, including but not limited to assessing the overall risk of a particular inmate to commit acts or self-harm or otherwise violent acts (such as fighting); or otherwise storage of alarm history for record keeping purposes (e.g., to evidence health and safety of inmate during incarceration). In this embodiment, as shown in FIG. 23, the archive screen shows for the cell (e.g., G1) the date and time of an alarm, alarm type (e.g., blood detection, fore, multiple presences, breathing rate anomaly, seizure or the like) and the operator/user who acknowledged the alarm. Accordingly, system 900 in this embodiment further includes data storage capabilities, as described elsewhere herein, by which system 900 can store alarm data for archiving purposes, and through which system 900 can recall such data when requested to do so via GUI 950 by the operator. In this embodiment, the archive screen further allows an operator to review the data from specific alarms, which may include unidentifiable data in some embodiments (but is not limited thereto). Some embodiments may, for the purposes of minimizing data to be stored, store only compressed data or certain data types, as predefined or as defined by an operator on a case-by-case basis.

Indeed, therefore, in some embodiments the systems and methods disclosed herein provide an unsupervised, non-contact event/risk detection system and methods, which further provide for storage, recall and/or learning from earlier alarms.

Exemplary System(s) without DVS

As noted above, embodiments of the systems and methods disclosed herein may be specifically configured to provide harm prevention monitoring systems and methods which do not require the inclusion of a DVS subsystem, or any associated DVS algorithm, as will now be described. Notably, in this embodiment of the system excluding the DVS component, other subsystems may be specifically configured to replicate or at least partly replicate the performance/operation of the DVS component. Exclusion of the DVS component may be suitable for some applications of the system for various reasons, including but not limited to, reducing system complexity, reducing system cost and/or improving system accuracy and/or confidence.

In this particular embodiment, seizure detection is developed for the RGB-D subsystem (e.g., 908) to replace the DVS detection (to complement it, in other embodiments), since in embodiments employing DVS, differentiating activities using only the DVS data can be difficult. In this embodiment, results using the human action recognition algorithm (CTR-GCN action classification network, e.g., 910) yielded successful detection of a simulated seizure at approximately 72.7% accuracy when tested with all other actions.

In various embodiments, other joint detection algorithms may be implemented, which show improved performance over the Kinect™ Azure™ joint detection, for example. It is to be appreciated that in the case where an inmate is lying down, or very close to a wall, the depth sensor (e.g., 908) has difficulty accurately predicting the joints of the inmate. The resulting joint predictions are not typically accurate and thus the action classification is severely impacted. In other embodiments, the other methods for joint detection use IR and thermal images to predict 2D joint coordinates of the inmate. These joint predictions should not be impacted by missing or erroneous depth data and are expected to perform significantly better for certain cases, specifically lying down compared with seizure. Test data, however, revealed that similar seizure accuracy was obtained using these algorithms, as compared to the Kinect™ Azure™ joint detection. In particular, as noted, Kinect™ Azure™ joint detection (St-GCN++ w/ Azure™ Kinect™ Joints) simulated seizure detection accuracy of 72.7%; whereas Alpha Pose 2D joint algorithms using IR (St-GCN++ w/ Alpha Pose 2D joints with confidence values (IR)) and thermal data (St-GCN++ w/ Alpha Pose 2D joints (Thermal)) yielded 63.6% and 66.2% accuracy, respectively. Accordingly, based on at least the foregoing, a combination of other sensors may perform as well as the DVS, thus affording its exclusion in this embodiment.

In some embodiments, fighting detection is developed for the RGB-D subsystem (e.g., 908) to replace the DVS detection (to complement it, in other embodiments), since in embodiments employing DVS, differentiating activities with similar levels of activity (for activities that should and should not generate alarms) using only the DVS data can be difficult. As such, the RGB-D subsystem in this embodiment was also used to detect fighting, and a two-person action recognition algorithm was specifically developed based on the human action recognition algorithm (CTR-GCN action classification network, e.g., 910). In this embodiment, results using the CTR-GCN model yielded successful detection of simulated fighting. In particular, the confusion matrix for a binary classifier that detects fighting when two inmates are present in the target area is as follows: for normal class, 82.4% precision and 92.4% recall; and for fighting class, 90.2% precision and 78.1% recall. Notably, this algorithm may provide a basis to develop detection of fighting between more than two inmates or the like.

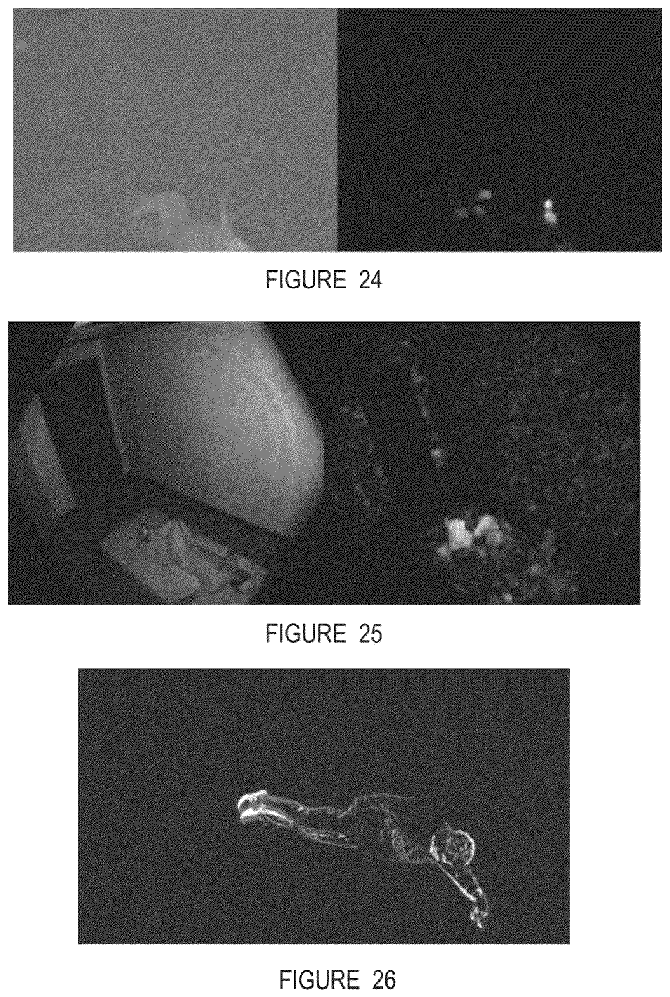

In some embodiments, the DVS components (i.e., DVS sensor and related algorithm(s)) are replaced with data from image-based sensors in the system, and more specifically optical flow detection from one or more cameras of the system having a wide dynamic range. Indeed, in some embodiments, optical flow calculations may be employed using both IR and thermal images to exclude DVS from the system (or to complement it, in other embodiments). Put differently, the insights gained from DVS sensors optical flow data may be recreated using one or more other sensors of the system to replicate the dynamic range of the DVS. For example, in one embodiment, the system or any one or more subsystems calculates a dense optical flow following Gunnar Farneback's algorithm, wherein the dense optical flow estimates motion using both a magnitude and direction, and every pixel in two consecutive images. To further illustrate such an embodiment, comparative data of various data is shown in FIGS. 24 to 26, as will now be described.

In FIGS. 24 and 25, respectively, a thermal imagery and IR imagery is shown in the left, with the complementary optical flow output of each on the right. This data was obtained during a simulated seizure. FIG. 26, on the other hand, shows DVS data obtained during a simulated seizure, for comparison purposes with FIGS. 24 and/or 25. Notably, because of the interference caused by the IR camera, the images could not be captured at the same time and are from two separate data collection sessions, although sufficiently illustrative for exemplary purposes. In FIG. 24, which shows the thermal image (left) of the simulated seizure alongside the optical flow output (right), it is clear that the optical flow output indicates the areas where the inmate is moving, at their extremities, while the rest of the scene remains unchanged. Similarly, in FIG. 25, which shows an IR image of the simulated seizure (left) alongside the optical flow output (right), it is clear that the optical flow output captures the motion but with a lower resolution. Additionally, the optical flow output image is (relatively) heavily impacted by artifacts-a result of the amplitude modulated active IR projection used to calculate the depth image. Therefore, the IR images alone may not be suitable for optical flow output (to replace DVS, for example) without removing these artifacts. In FIG. 26, which shows the DVS data during the simulated seizure, it is clear that the motion is captured across the inmate's entire body (hence why, in some embodiments, use of DVS 25 may be useful). Comparatively, the resolution of the DVS in FIG. 26 is much higher than either the thermal or IR sensors, hence, the motion is more finely captured. Nonetheless, it is clear from comparing the images in FIGS. 24 to 26 that the same motion is captured in all three images; although the level of resolution and artifacts are (somewhat substantially) different. Thus, in some embodiments, DVS may be excluded from the system where thermal and/or IR sensors form part of the system and the imagery thereof can be represented as optical flow output(s), as shown.

Based on the foregoing exemplary comparison of motion detectable by different data types and/or data processing, several non-limiting reasons for the exclusion of DVS from some embodiments may be identified. In particular, with regard to artifacts with IR and depth images, the active IR sensor used in the depth camera may create artifacts within the dynamic range of the DVS. In some embodiments, to operate the DVS in the same environment as the depth sensor, a filter is used with the DVS camera lens, which typically reduces the dynamic range of the DVS to that of an RGB camera. As such, other RGB cameras and/or RGB-D cameras forming part of the system may be sufficient. With regard to cost, and as noted above, developing the usage of the other sensors (as described above) to at least produce workable output of motion may render the DVS redundant in some embodiments, thus allowing exclusion thereof to reduce overall system cost. With further regard to the data redundancy offered by using optical flow algorithms (as discussed above), since the above embodiment evidences the extraction of data like the DVS data by running an optical flow algorithm on the IR, thermal and/or RGB data, use of DVS may be rendered redundant for some embodiments. Therefore, in different embodiments, algorithms used for the DVS can be implemented on the optical flow output from another data type or types.

### Exemplary Blood Detection Framework

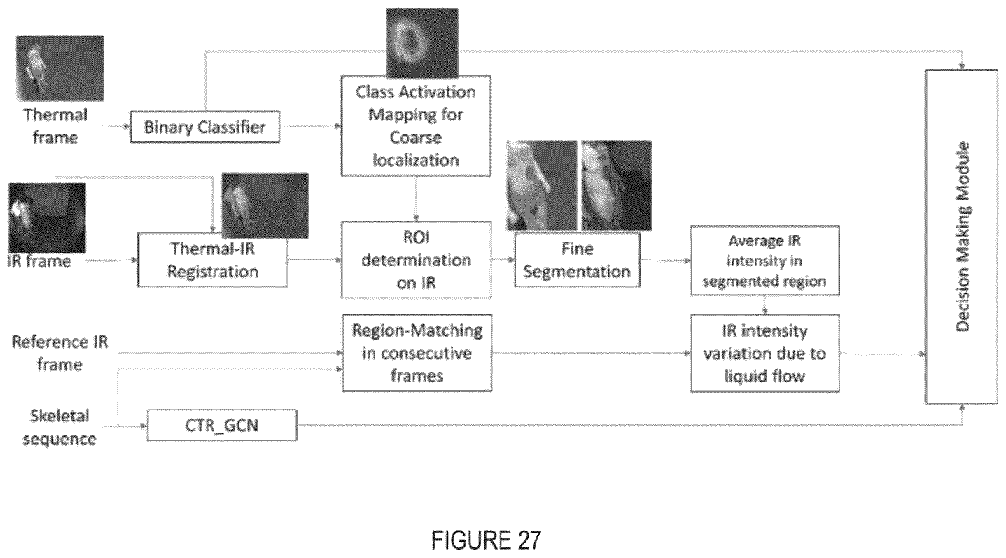

A further embodiment of the bleeding/blood detection framework is shown in FIG. 27. This framework may form part of an ALSM system architecture, or a simpler system architecture configured specifically for monitoring for bleeding or the presence of blood. In this embodiment of the bleeding/blood detection framework (or algorithm), the design integrates the use of two thermal cameras and two active IR cameras. The IR cameras are already in use in this embodiment through a Kinect™ sensor, to capture the geometry of the cell as measuring the time of flight of emitted pulses and the same reflected signals are used for IR image formation. The warmer temperature of blood compared to, for example, skin surface, clothing (in many cases), environmental objects and flooring, makes it detectable and distinguishable with thermal cameras in many scenarios. In addition and/or in the alternative, the other signature that can be relied on to distinguish blood from other liquids, is its spectral response within the spectrum of the emitted pulses. For example, experiments run by measuring the spectral response of blood, coffee, tea, milk, water, and juice on a variety of surfaces concluded that blood reflects a very small portion of the emitted 850 nm pulses with a reliable margin, in comparison with all other liquids, which results in low intensity of blood on IR images. The combination of the liquid properties, temperature and IR emittance are used in this embodiment to reliably detect blood using the thermal and IR cameras.

As noted, FIG. 27 shows the overall architecture of this embodiment of the bleeding/blood detection framework. To reduce the computational cost of this embodiment, a binary classifier is trained on thermal images from the scene to determine if any warm liquid is “spilling” in the scene or not. This prevents all other system modules from running if no liquid is detected. If a warm liquid is detected, the framework continues to identify whether the liquid is blood or not, using a variety of algorithms relying on both thermal and IR images, as will be briefly described next.

In this embodiment, an initial stage involves binary classification and coarse localization of liquid spill in thermal images. The binary classifier in this embodiment is a Deep Convolutional Neural Networks (DCNN) trained to decide if warm liquid is “spilling” in the scene or not. Training was performed on a total of 76,500 thermal images including a balance of “containing bleeding (positive)” and “without bleeding (negative)” classes. Notably, training in this embodiment included a relatively wide temperature range of 30 to 40° C., to ensure that the classification was robust so as to accommodate the thermal drift effect over time and/or a rapid temperature drop in blood (e.g., when spilling onto cold surface). Training in this embodiment also included liquid spills (e.g., simulated by warm water) over different surface types (e.g., cloth, wrist, floor, mattress) as well as different clothing materials with different colours, to reduce any possible bias. Various different backbone architectures may be workable in different embodiments. In this instance, various different backbone architecture (e.g., Resnet 101, Renet50, MobilenetV2) with different input image resolutions of 224×224 and 480×640 were trained to determine the classification accuracy for this application (indeed, different backbone architectures may be workable in different embodiments). Generally, a larger input image resolution afforded improved accuracy, thus 480×640 is preferred over 224×224 in this embodiment. In this embodiment, Resnet 101 was ultimately utilized, having the deepest architecture and outperforming other networks tested. To prevent any sort of bias in the dataset, at least in part, the binary classifiers were repeatedly trained and tested them with Local Interpretable Model-Agnostic Explanations to identify various factors that influence the decision of the network. Accordingly, the influence of all non-relevant factors is eliminated by collecting more data. Thus, to improve the robustness of the binary classifier in this embodiment, all positive image sequences were manually annotated by determining the frame at which water spill starts and adding them to the positive folder, whilst the negative class was created by manually choosing and adding the negative samples to the corresponding class.

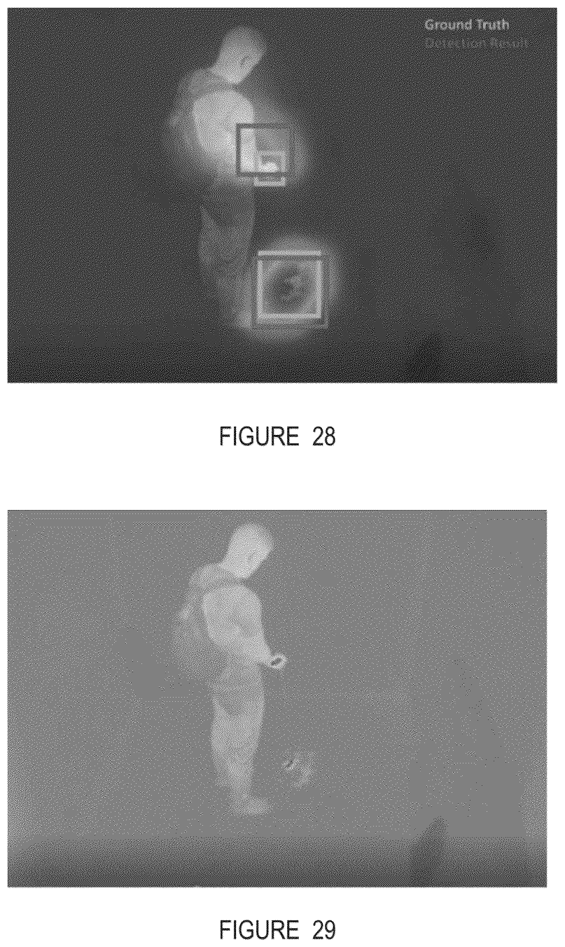

In this embodiment, the binary classifier is then used to compute a Gradient-Based Class Activation Map (Grad-CAM) for positive image classes. The Grad-CAM identifies the important regions of the thermal image, leading the DCNN to classify the input as “containing liquid spill”. In this embodiment of the coarse region of interest (RoI) determination, data annotation is performed using the Video Labeler tool of Matlab in the form of bounding boxes. Notably, in this embodiment, backbone architecture Resnet 101 was utilized for the coarse RoI detector based on the average Intersection over Union (IoU) for the test dataset being significantly higher than other backbone architectures (calculated by measuring the intersection between the detection result and the ground truth divided by the union of the two regions). Since the coarse RoI detection module is a weakly supervised approach, data annotation is performed only for evaluation purposes in this embodiment. FIG. 28 shows one embodiment of a processed thermal image of a bleeding event (e.g., self-cutting), illustrating the coarse segmentation result this embodiment of the thermal image analysis algorithm. As shown, the detection result of the course segmentation is somewhat larger and/or offset from the ground truth in this embodiment. Image regions with an activation value higher than an empirically determined threshold of 0.7 in this embodiment are further analyzed using the fine segmentation network.

In this embodiment, the next stage thus involves fine segmentation of the liquid (i.e., blood), relying on the result of the binary classification and coarse segmentation. For the fine segmentation network, all the training, testing and validation sets are annotated at pixel level using the Video Labeler tool of Matlab (although other embodiments may utilize other tools for pixel level annotation). The fine segmentation network precisely determines which pixels in the thermal image correspond to the detected liquid. FIG. 29 shows a processed thermal image of the bleeding event shown in FIG. 28, illustrating the fine segmentation result of this embodiment of the thermal image analysis algorithm. As compared to the course segmentation result of FIG. 28, the fine segmentation result of FIG. 29 is more localized and indeed, allows for more precise determination of which pixels correspond to warm liquid. This fine segmentation in this embodiment allows to precisely track the liquid in the thermal and the active IR camera to check its spectral response. For the fine segmentation network, several different segmentation methods may be workable in different embodiments, including for example DeepLabv, U-net and SegNet. In this embodiment, the Deeplabv3 architecture was employed with a Resnet 101 backbone pretrained on the in-house dataset, particularly since this segmentation network offered a significantly improved average IoU for the test dataset over the other networks tested (although in other embodiments, other networks may be workable). The segmentation network gets the features extracted by the binary classifier and then, in this embodiment, applies an Atrous Spatial Pyramid Pooling to it to extract multi-scale features from the region of interest. This layer is enables the network to detect even small regions that contain any liquid in this embodiment.

In this embodiment, the next stage of the bleeding/blood detection framework pertains to thermal-IR image registration. In this embodiment, each cell has each thermal camera mounted on a bracket along with a Kinect™ having an IR camera in use for bleeding detection. For each thermal-IR camera pair, the framework computes a transformation matrix, allowing the identification of pixel to pixel correspondence between IR and thermal images. As such, the output of the fine segmentation network can be tracked in the IR camera to check for any rapid intensity changes along the frames due to a liquid “spill”.

In this embodiment, region of interest matching and tracking (in consecutive frames) follows the thermal-IR image registration. In particular, whilst blood appears with lower intensities compared to other liquids in the IR camera of Kinect™, the exact spectral response of a surface contaminated with blood depends highly on the spectral properties of the material underneath. Consequently, in this embodiment, the intensity of the detected region is tracked along the frames to check for intensity changes due to liquid spill. In this embodiment, if the detected region is on static background objects (e.g., bed, floor, desk or the like), the IR video frames stored in a sampling buffer are directly used to measure the intensity changes. If the detected region is on a moving background object (e.g., an inmate), the skeletal data is integrated to track the moving body part over which liquid spill is detected and decide if liquid spill results in any significant intensity changes. Notably, monitoring for intensity changes may be beneficial over monitoring for pure intensity in some embodiments.

Based on the foregoing description of this embodiment of the bleeding/blood detection framework, it should be noted that the decision-making module(s) relies on multiple factors to trigger an alarm. These factors include the outcome of any one or combination of: the binary classifier, the temperature of the detected liquid, the intensity variation as a result of liquid spill and the outputs of the activity recognition module within a time window, although other factors may be relied on in addition or alternative thereto in other embodiments.

### Exemplary Motion Detection Framework

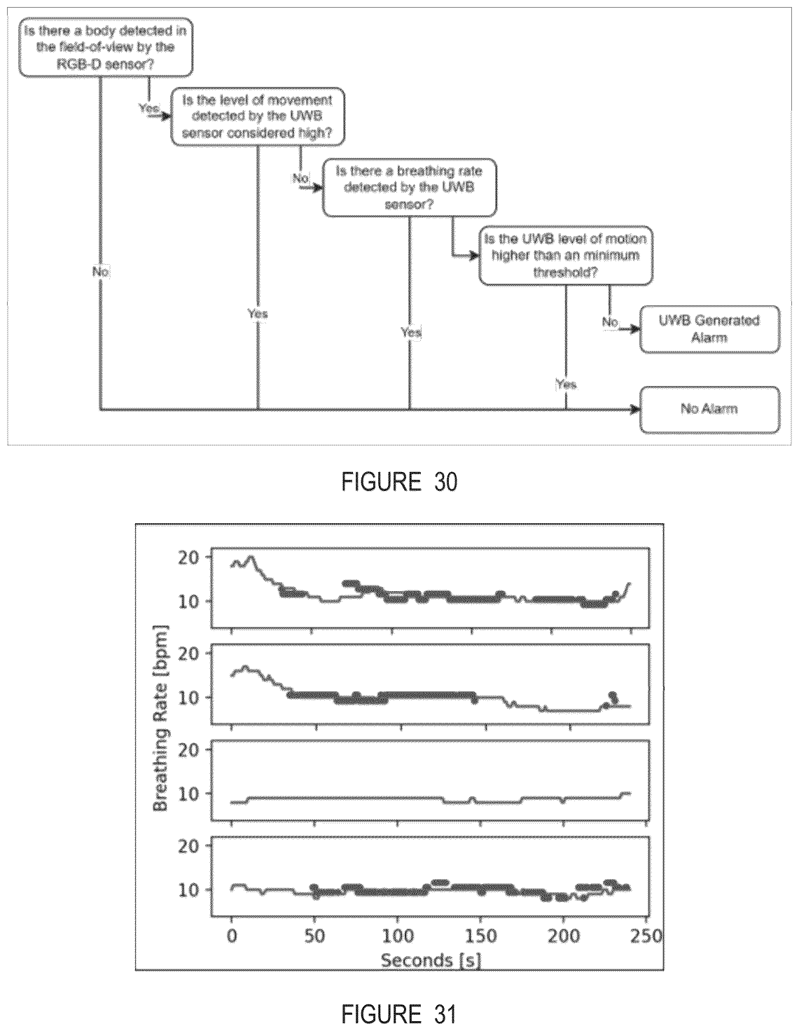

A further embodiment of a motion detection framework is shown in FIG. 30. This framework may form part of an ALSM system architecture, or a simpler system architecture configured specifically for monitoring for breathing rate, heart rate and/or level of motion (i.e., movement index) in a target area (e.g., cell). In this embodiment of the motion detection framework (or algorithm), the framework relies on data from a UWB radar with other sensors (e.g., RGB-D) to detect, for example, if there is someone in the field-of-view and whether the level of motion is consistent with someone who has stopped breathing. Thus, the embodiment of FIG. 30 uses multiple sensor inputs and calculations to determine whether an alarm should be generated. It is to be appreciated, however, that in certain embodiments, another sensor or sensor combination may produce sufficiently accurate results (for example, RGB-D data with sufficient processing may be sufficiently accurate to exclude radar data acquisition and processing for breathing detection).

In this embodiment, the three radar sensors (e.g., Xandar™ Kardian™ UWB radar sensor, Vayyar™ UWB radar sensor or the like) were installed in a cell at positions directly above the bed, perpendicular to the bed and parallel to the bed. These radar sensor positions were selected to direct the radar at the chest of an inmate sleeping or lying on the bed. Notably, in this embodiment, the radar sensors are specifically configured and/or installed to monitor the chest of the inmate in different sleeping positions, including the most common sleeping positions (e.g., back, front, right side and left side). In this embodiment, before carrying out the framework of FIG. 30, one or more calibration steps is performed. In particular, calibration is performed with no moving objects in the FOV of the radar, only fixed objects (e.g., furniture) so as to allow the determination of where fixed objects are in the scene prior to testing/monitoring.

Notably, when testing the accuracy of the radar sensor alone, compared to a ground truth measurement for heart rate and breathing rate obtained with a Hexoskin™ vest, taken as various sleeping positions, overall breathing rate accuracy in the order 1.08 was obtained. This breathing rate accuracy metric is defined as the average absolute difference between the values recorded using the Hexoskin™ vest and the values determined using the UWB radar. The accuracy is calculated over a period of time, taking the average of a series of values taken at one second intervals. Further notable, the overall breathing rate reliability of the radar is only approximately 38%. The breathing rate reliability metric is defined as the percentage of time that the UWB radar device gives a result for the breathing rate. These two metrics are only calculated when the subject is motionless (aside from sleeping-related motion) in this embodiment, as the device is not rated to measure breathing rate during motion. Based on at least the reliability metric, reliance on the level of motion detected in the cell by the UWB radar is crucial in some embodiments, rather than just the breathing rate when determining whether an inmate is breathing or not. In some embodiments, the movement index may be used as an indicator for vital sign presence. Furthermore, in some embodiments, the motion detection framework may be specifically configured to accommodate sensor positions known to be difficult to accurately detect breathing rate with radar, such as by additional training and/or supplementation with data from other sensor(s). FIG. 31 shows specifically the breathing rate detected by the UWM radar sensor above the bed in this embodiment, plotted in thicker blue lines, as compared to the ground truth breathing rate as detected by the Hexoskin™ vest, plotted in thinner red lines. In FIG. 31, from top to bottom, the sleeping positions comprise: back, front, left side and right side. As shown, radar sensing in this embodiment provides one mechanism for detecting breathing rate during sleeping, at least when the inmate is sleeping in the exemplary cell on his/her back, front and right side. Although no data from sleeping on the left side is shown for this radar sensor (positioned above the bed), another radar sensor positioned at a spaced apart position (for example, parallel to the bed and/or perpendicular to the bed) may detect breathing rate better (data not shown).

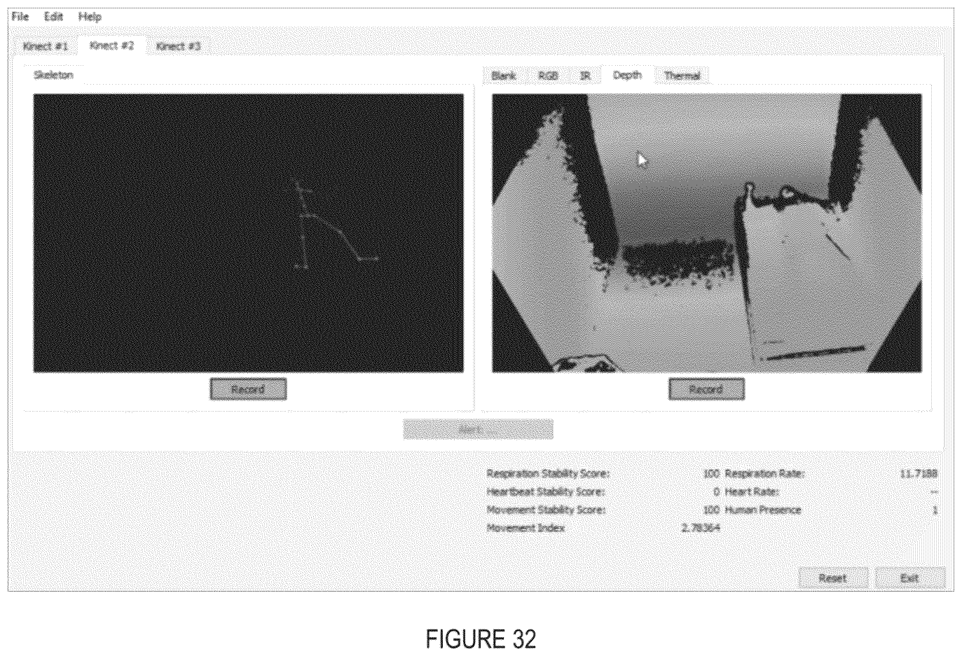

In this embodiment, as shown in FIG. 32, breathing rate, heart rate, movement index, occupancy (human presence), and the stability scores for all metrics, is displayed to the operator via a GUI. Any other embodiments, any one or combination of these metrics may be displayed or hidden. Consistent with the motion detection framework of FIG. 30, if a breathing rate anomaly is detected (based on predefined thresholds), an alarm is generated to notify the operator of same.

In other embodiments, the motion detection framework may further define a motion index for classification of different movement levels. In one embodiment, the motion index may specifically distinguish and/or allow identification of any one or combination of: empty room; no vitals person in room; HR present no BR person in room; breathing rate detected (low movement); low motion; and high motion.

### Exemplary Fire Detection Framework

In one embodiment, the fire detection framework (or algorithm) relies on thresholding thermal images to detect elements that correspond to small flames or fires. The framework of this embodiment is designed for small flames and fires that would not be detected by the smoke alarm. Specifically, the framework identifies flames from lighters and other small flames, such as lit cigarettes, which may pose fire hazards, or otherwise may be used for self-harm through burning. In this embodiment, traditional computer vision techniques were employed to detect when threshold temperatures (or intensities) were identified, thereby simplifying processing required (although in other embodiments, AI-based approaches may be workable).

In this embodiment, Teledyne Dalsa thermal cameras were employed, having approximately 0.3° precision when operating in 16-bit mode. This precision easily allows accurate detection of fire and flames if the object of interest is significantly large, this is defined as minimum size of a 3×3 grid for accurate detection. In some use cases, where the flame or fire is smaller than this, the framework allows for the detection of the risk but accurate temperature estimation may not possible depending on flame size.

In order to train the fire detection framework, which is based on binary classification in this embodiment, a dataset containing images of a variety of small flames produced by several different types of lighters (test images), as well as several control images not containing any flame(s), was generated. The sensitivity of the fire detection framework on a test dataset was 87.2%, while the specificity was 99.9%. The sensitivity is impacted by cases where the flame is very small or partially obstructed/occluded by the lighter or the person holding the lighter, for example, and as such spaced apart arrangements of sensors (with different FOVs) can attempt to avoid these obstructions/occlusions. Put differently, basing the fire detection framework on multiple sensor data (from similar and/or distinct sensor types) reduces the number of false negatives in some embodiments.

It is to be appreciated that in other embodiments, in addition to thermal intensity (or temperature) thresholding, other metrics or parameters can be used to detect flames or fire, including area, circularity, convexity, and inertia. Optimal values for these parameters were determined using the acquired dataset.

It is to be appreciated that several aspects of the instant disclosure are described, for simplicity, with reference to a system having hardware and software components. However, these descriptions may be used to convey explanation of associated methods which are not necessarily dependent on that particular hardware and/or software components, but instead may be implemented by any number/type of components.

It is to be appreciated further that whilst the above embodiments are largely directed to “harm prevention”, embodiments of the system and methods may be operable to detect events/risks/scenarios after the fact, due to processing times required or otherwise, and depending on the use-case. Otherwise, embodiments may be operable to detect other gestures, motions, actions, events or the like, which are not specifically limited to “harm prevention” applications, nor indeed limited to the identification of “anticipated harm scenarios” as such. For example, certain embodiments may be directed to security applications, such as the detection of prohibited objects, actions, gestures or events, in secure environments. For example, the systems, methods and/or frameworks disclosed herein may be operable to detect the presence of a weapon (firearm, sharp object or the like) in a commercial bank environment. In other examples, the systems, methods and/or frameworks disclosed may be operable to detect the gesture or action of lock tampering in a safe room, opening a safe at an unexpected time or keeping the safe open for an unexpected duration, or the like. In such examples, the predefined scenario for which the risk is assessed may be, for example, a bank heist or hostage situation, a safe break-in, safe theft or the like. As such, in such applications, the systems, methods and/or frameworks disclosed herein may provide for security surveillance systems. In other examples, the system may be deployed in a vehicle or transportation system such as a bus, subway, train, airplane or car, where certain actions or behaviours are prohibited or discouraged and where human supervision may be not be available. This may, for example, be the case for autonomous vehicles in which anonymized monitoring may be deployed to detect such activities. In yet other examples, the systems, methods and/or frameworks may be deployed to privately monitor patients or inmates on an ongoing basis, to categorize levels of aggression associated with patients, levels of depression associated with patients, or the like, these then being considered the anticipated harm scenarios, based on the ongoing recognition of characteristic features (e.g., posture, gestures, actions). In various embodiments, such monitoring and detection of objects, actions or events (designated as prohibited or discouraged in the designated environment, although not necessarily so) may maintain or protect the personal privacy of the individual(s) in the designated environment, as described herein.

Indeed, certain embodiments of the systems, methods and/or frameworks disclosed herein may be specifically configured so as to maintain the privacy of the individual(s) monitored in the designated environment. Here it is to be appreciated that certain embodiments may extend to designated environments where individuals come and go at free will (e.g., a bank, a supermarket, a school, a tourist attraction, a park or the like) and therefore, in such embodiments, individual privacy rights (particularly in the lack of any explicit consent to recordal of private or personal data) may be of concern. Certain embodiments may provide for the surveillance of designated environments for the detection of certain scenarios (typically predefined and prohibited) whilst ensuring strict personal data privacy.

Some embodiments provide specifically for systems, methods and/or frameworks within which the data acquired of the designated environment, including the current state of the individual, is automatically or near instantaneously anonymized. Where a plurality of data types are acquired, all such data types are anonymized, appreciating that certain data types (e.g., radar, IR at lower resolutions) may be inherently anonymized to some extent. Here, this automatic or near instantaneous anonymization of data acquired of the designated environment and/or the individual(s) therein may comprise removing from the data acquired any personally identifiable information (PII) for the purposes of privacy protection. Such PII may include any one or combination of: facial features of an individual, hair colour and/or texture of an individual, eye colour of an individual, a name tag of an individual, personal piercing or markings (e.g., tattoos, birthmarks, unique moles or pigmentation, etc.) of an individual, a voice of an individual, an identity document of an individual, a vehicle license plate of an individual, clothing of an individual, or the like. Further types of PII may be applicable to certain applications and are thus not limited to the foregoing list. Different applications may require different levels of data anonymization and/or different sensitivity, and thus certain embodiments may allow operators or users to set a predefined PII threshold, for example, which defines what PII may be retained, what PII should be obfuscated and/or what PII should be discarded, for certain data types. In certain embodiments, the sensors or sensor array itself is configured so as to anonymize the data acquired, before communicating such data to a CPU, transient data storage or other componentry of the system. In such embodiments, certain data is considered to be anonymized at the source.

In one specific embodiment, there is provided a privacy-maintaining system for automatically monitoring one or more individuals in a designated environment to identify a risk of harm, specifically whilst maintaining privacy of the one or more individuals. The system comprises a sensor array arranged within the designated environment to provide complementary views thereof and configured to acquire data of a plurality of data types representative of a current state of the one or more individuals in the designated environment. In this embodiment, the sensor array includes a depth-enabled image-based sensor which in use captures image-depth data of the designated environment. The system also comprises a control interface configured to communicate with the sensor array and a remote device. The system further comprises a digital data processor in communication with the sensor array and the control interface. The digital data processor is configured to execute digital instructions to automatically: via the data acquired from the sensor array, extract in real-time a characteristic feature of the current state of the individual(s), digitally compute using the characteristic feature the risk of harm with respect to an anticipated harm scenario at least partially by implementing a human action recognition process on the characteristic feature, in this embodiment, and upon the risk of harm corresponding with the anticipated harm scenario, communicate via the control interface to the remote device an alert corresponding to the anticipated harm scenario. Notably, complementary methods and frameworks are also envisaged but are omitted for the sake of brevity. For the same reason, features shared with the systems, methods and/or frameworks discussed above are not repeated here.

In this embodiment, the one or more sensors forming part of the sensor array are configured so as to automatically anonymize data at upon acquisition (or at the source), using one or more of the predefined data anonymization processes described below. For example, a sensor may be configured to automatically extract or track skeletal projections or data, without recording any image data. In another example, a sensor may be configured to automatically obfuscate PII such as facial or body features using contouring and/or segmentation. Further details on such predefined data anonymization processes, which may also be implemented at or integral to the sensor(s) themselves, are provided below. In some embodiments, the digital data processor is further configured to execute digital instructions to automatically anonymize any one or more of the plurality of data types acquired from the sensor array to provide anonymized data. In particular, the digital data processor is so configured when the one or more sensors from the sensor array is not configured so as to automatically anonymize the data acquired.

It is to be appreciated that removing from the data acquired any PII may comprise different removal step(s) for each data type acquired, although similar step(s) may be employed for one or more data types. For example, removal of PII from image or video data may differ from removal of PII from audio data or radar data. Furthermore, depending on the data acquired, two or more techniques for PII removal may be employed in combination in some embodiments. Yet further, as noted, this removal of PII may differ in various embodiments based on PII thresholds set for the particular application or threat level. In some embodiments, the digital data processor removes the PII based on a predefined data anonymization process, which may or may not include the predefined PII threshold(s) set. As noted, in other embodiments, the PII is removed at the sensor(s).

In some embodiments, removing from the data acquired any PII by implementing the predefined data anonymization process may comprise extracting and/or tracking from the data acquired the skeletal projection (or data or frame), as described elsewhere herein, and (permanently) discarding any image or video data from which such skeletal projection was extracted, for example. Accordingly, the data fed to the human action recognition process or similar model comprises skeletal tracking or projection data (e.g., joint identifiers and associated coordinates), which provides the motion of the individual(s) devoid of any PII. By discarding the image or video data after skeletal projection extraction or tracking, and retaining only the skeletal tracking or projection for analysis, the system, method and/or framework may present a privacy protective or preservative system which safeguards the individual(s) privacy whilst still allowing for the detection of certain predefined characteristic features (and in turn, actions or events) and if necessary, generating an appropriate alarm. Furthermore, no storage of the image or video data is required for recordkeeping, and thus no long-term compliance with private data storage protocols is required. Storage of skeletal tracking or projection data, if stored, indeed preserves the privacy or the individual(s), whilst presenting the further benefit of requiring less storage space as compared to image or video data. Notably, in some embodiments, this automatic or near instantaneous discarding of PII or data associated therewith ensures that even if the system or framework were to be hacked by a third party, for example, the third party would not obtain the PII from the acquired skeletal data, if stored. The anonymized skeletal data stored, if stored, is thus privacy protected data devoid of a predefined threshold of PII.

In other embodiments, removing from the data acquired any PII by implementing the predefined data anonymization process may comprise identifying or recognising PII in the data acquired (or potential PII) and automatically or near instantaneously blurring such PII in the data acquired. Here, the PII may not be recognized or identified as such, but rather bounds or contours associated therewith may be identified. In such embodiments, a greater level of differentiation on levels of anonymization may be possible (as compared to, for example, skeletal data extraction where all image or video data is discarded). For example, the system, method and/or framework may comprise a user-agnostic facial blurring model which is operable to identify facial bounds associated with an individual and blur the data content within such facial bounds to anonymize the acquired data. Such facial blurring may not remove from the acquired data attributes such as personal markings or piercings. In some embodiments, where data includes image or IR data, facial blurring may be achieved by adjusting the pixelated content within the facial bounds to obfuscate any identifiable facial features. To provide another example, the system, method and/or framework may comprise a user-agnostic body silhouetting model which is operable to identify bodily bounds (optionally including clothing) associated with an individual and blur the data content within such bodily bounds to anonymize the acquired data. Such bodily blurring may provide the silhouette of the individual(s), allowing detection of characteristic features, whilst protecting the PII of the individual(s). To provide another example, the system, method or framework may comprise a user-agnostic tattoo blurring model which is operable to identify tattoo bounds associated with an individual and blur the data content within such tattoo bounds to anonymize the acquired data. Accordingly, in any such embodiments, the data fed to the human action recognition process comprises anonymized data representative of the bodily motion of the individual(s), devoid of a threshold of PII.

In other embodiments, removing from the data acquired any PII by implementing the predefined data anonymization process may comprise extracting or tracking from the data acquired optical flow output, as described elsewhere herein, and (permanently) discarding any image or video data from which such optical flow output was extracted, for example. It is to be appreciated that such optical flow output may provide the pattern of motion of the individual(s), devoid of any PII. This technique may exhibit benefits similar to those as described for the skeletal extraction above, including automatically anonymizing the data and ensuring that any data stored, if any, is devoid of PII.

In other embodiments, removing from the data acquired any PII may comprise any other data obfuscation technique which can be restricted to such PII without obstructing motion data required for the detection of characteristic features and/or assessing the risk of the predefined scenario. For example, other object recognition and/or image processing techniques are envisioned to be employable in other embodiments. A few nonlimiting examples of image processing techniques that may be employed in other embodiments to obfuscate or remove PII from data acquired include: feature extraction, image segmentation, thresholding, edge detection, pixelation and/or the like. In yet other embodiments, where the data includes audio for example, removing the PII may comprise any audio obfuscation technique for removing predefined PII, such as identifying and blocking personal identifiers (or non-generic words generally) from an audio feed, altering the pitch, vocal colour, reverb or the like of an audio feed to produce a synthetic audio feed, or the like.

In yet other embodiments, removing from the data acquired any PII may comprise merely confirming that one or more of the data types acquired are inherently anonymized. In some embodiments, radar data is considered inherently anonymized (or inherently anonymized upon acquisition) and therefore confirmation of this data type is sufficient to conclude the removal of PII for this data type. Whilst radar data may not inherently provide PII, it is to be appreciated that this data type may be useful in other methods or frameworks described herein, such as to identify the presence of one or more individuals, to identify the activity level of one or more individuals (e.g., sleeping vs awake) or the like. In some embodiments, if one data type is thermal or infrared (IR) data, and this IR data is captured at a resolution insufficient to identify PPI therefrom, the IR data may be considered inherently anonymized (upon acquisition) and thus merely confirming this data type is sufficient to conclude the removal step. Conversely, where the IR data captured is of a high resolution such that detection of facial features or markings is possible, removing from the IR data any PII may comprise any one or combination of the techniques described elsewhere herein. Notably, the same may be applicable to dynamic data acquired by a dynamic vision sensor, for example.

In some embodiments, anonymizing the data acquired (whether at the sensor(s) or at a central processing unit) is such that only data required for detecting or identifying the characteristic feature forms part of the anonymized data fed to the human action recognition process. Whilst anonymized motion data may be relevant for human action recognition in general, it is to be appreciated that other data categories may be equally relevant (e.g., presence, positioning, speed, or the like). Only data required for identifying the characteristic feature is processed and/or stored which, in some embodiments, ensures that the data of the individual and/or environment is indeed privacy protected. For example, where image data is acquired but skeletal projections extracted therefrom are sufficient for identifying the characteristic feature, the image data may be (permanently) discarded and the skeletal projection(s) retained, if necessary. Thus, in some embodiments, anonymizing the data acquired and detecting the characteristic feature (gesture, action or the like) may be executed concurrently or simultaneously.

Yet further, in some embodiments and as mentioned, acquiring the data and anonymizing the data acquired may occur concurrently or simultaneously, or at least near instantaneously. For example, radar data acquired or low-resolution thermal data acquired may be considered concurrent acquisition and anonymization of data. In another example, skeletal or skeleton tracking may be employed in real-time such that the motion and/or posture of the individual(s) is tracked by the sensor but no other features, including any PII, is tracked, analyzed, recorded or stored.

In some embodiments, detecting the characteristic feature from the anonymized data comprises executing a characteristic recognition process to extract in real-time (or near instantaneously) the characteristic feature. In some embodiments, the system comprises a machine learning-based architecture configured to execute this characteristic recognition process, as previously described herein. It is to be appreciated that this characteristic recognition process may be operable, in various embodiments, to recognize patterns in the anonymized data based on predefined training data fed to the ML-based architecture. For example, if one of the characteristic features of interest is a sequence of motions and/or gestures undertaken by an individual during opening a safe, the training data fed to the ML-based architecture (specifically, model) may comprise various individuals undertaking similar actions such that the characteristic recognition process is trained to recognize similar patterns by other individuals in the designated environment. Here it is to be appreciated that the ML-based architecture may be trained on anonymized data (e.g., postural and/or gestural projections, such as skeletal projections or silhouetted projections) such that the characteristic recognition process itself does not tend towards any ML bias (for example, that men typically open the safe). Notably, the predefined harm scenario in such an example may include that the safe is being opened and/or that the safe is being tampered with. For example, one exemplary embodiment may monitor and store data each time it is recognized that the safe is opened, and may generate an alert when the risk of safe tampering exceeds or sufficiently matches, maps to, a predefined risk threshold, or otherwise corresponding with a risk profile. Such alert generated may be communicated to the remote device without identifying the one or more individuals associated with the predefined scenario.

In some embodiments, the characteristic recognition process specifically comprises a human action recognition process (HARP). Such HARP may be trained on specific human actions or gestures expected or prohibited in the designated environment, and/or otherwise believed to be contributing factors to an anticipated harm scenario (e.g., depression). In some embodiments, the HARP may be operable to distinguish between different individuals in the environment and be trained on specific human interactions expected or prohibited in the designated environment. As such, more than one skeletal projection, facial or bodily border, or the like, may be extracted or detected in various embodiments.

The anonymized data may be stored on one or more digital storage devices, although such storage is not necessary in all embodiments. In one embodiment, the data stored is devoid of PII to the PII threshold defined for the particular application. In some embodiments, the data stored may be devoid of any image or video data whatsoever. In some embodiments, anonymized data is only stored in the event of an alarm or alert being generated. In other embodiments, only data representative of the occurrence of the alarm or alert is stored.

These privacy maintaining systems, methods and/or frameworks may be employed in any number of applications, as noted, and the models or frameworks employed (e.g., human action recognition process or model) may be configured or adapted to suit the particular application or designated environment. For example, predefined scenarios in a bank setting may range from the presence of a weapon (e.g., knife or firearm), to the presence of blood, to the presence of masked individuals, to the detection of recognizable hostage gestures (e.g., weapon to throat of bank employee or customer) to the like. The risk threshold or profile tolerable before an alarm is generated by the system, method and/or framework may also thus differ for different applications. For example, banks may generally require relative low risk thresholds to generate alarms even at risk of false negatives. Further, banks may define different risk thresholds or profiles for different predefined scenarios—for example, the detection of safe tampering having a higher risk threshold or profile (there being additional security features in place) than the detection of a hostage situation. As another example, a detention center may generally require at least a medium risk threshold to be reached before a psychologist is alerted of the potential depression or mood swing of an inmate. Notably, the computation of risk of the predefined scenario may refer to a risk of the predefined scenario having already occurred or being expected to materialize (i.e., reactively or proactively) in different embodiments.

In most embodiments, it is to be appreciated that the systems, methods and/or frameworks are configured to monitor a designated environment to identify characteristic features of individuals without identifying the individuals themselves, thus protecting the identity of the individual. Put simply, the focus is on identifying what the individual is doing, not who the individual is. Accordingly, in some embodiments, anonymizing the data acquired may include retaining only the minimum amount and/or scope of data necessary to identify what the individual is doing.

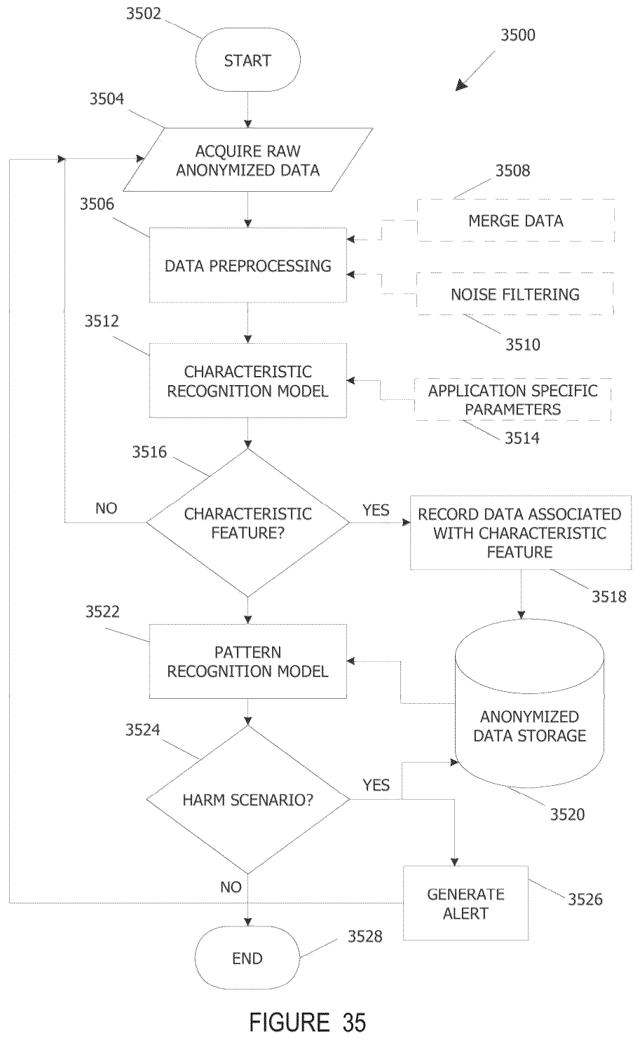

Turning now to FIG. 35, this figure illustrates one embodiment of the methodology employed in an exemplary privacy-maintaining system 3500 for automatically monitoring an individual in a designated environment to identify a risk of harm, as described above. Once the system starts at 3502, the sensor array arranged within the designated environment acquires at 3504 raw, anonymized data of the individual in the designated environment. In this embodiment, the raw data is specifically acquired in anonymized format, in that either the one or more sensors are inherently anonymous, and/or the internal data processing associated with the sensor(s) immediately anonymizes the raw data at the sensor(s) (i.e., at its source and/or upon acquisition). As previously noted, various sensors and/or camera systems may be configured as disclosed herein, so as to immediately acquire anonymized raw data of the individual in the designated environment or otherwise so as to immediately anonymize acquired raw data (e.g., by skeletal tracking, silhouetting, blurring certain PII, or the like).

The anonymized data then undergoes preprocessing. Such preprocessing would vary on the data type(s) acquired and may include, for example, merging of skeletal projections at 3508, filtering of noise at 3510, and/or the like. For clarity, any such preprocessing at 3506 is focused on clarifying the data feed and in no way attributes any PII to the data. After preprocessing at 3506, the preprocessed data is fed through a characteristic recognition model or process at 3512 which may be trained as herein described on anonymous datasets, and which may receive at 3514 further parameters specific to the designated environment or individual in it, for example. In this embodiment, the characteristic recognition model is configured to identify at 3512 from the anonymous data certain characteristic features, such as sleeping, sitting, exercising, reading, punching, strangling, digging, or the like, to name but a few examples. At 3516, the system determines whether one or more characteristic features are identified (or partially identified, as needed). If one or more characteristic features are identified, the system at 3518 records the anonymous data associated with the characteristic feature(s) and/or its recognition. Such recordal 3518 may include recordal of the raw or preprocessed data itself, or otherwise the recordal of an indicator of such characteristic feature recognition (e.g., sleeping from 9 pm to 6 am), at a remote anonymized data storage site 3520. If no characteristic feature is recognized by the model, the system 3500 continues acquiring 3504 and assessing data. In this embodiment, the preprocessed data and/or characteristic feature is next fed though a pattern recognition model or process 3522 which is configured to determine whether the characteristic feature(s) recognised at 3516 may equate to, map to, or otherwise be associated with, an anticipated harm scenario (which as noted, may not constitute “harm” as such). If an anticipated harm scenario is detected or identified at 3524, an alert is generated at 3526. Continuing the above example, if the pattern recognition model at 3522 determines that the individual is exhibiting a specific characteristic feature more frequently (e.g., sleeping more frequently than usual), for example, and this maps to a specific pattern of interest at 3522, this may be identified as an anticipated harm scenario at 3524 and an alert may be generated at 3526, and/or the anticipated harm scenario from 3524 may be recorded and stored at 3520. In this regard, it is to be appreciated that not all harm scenarios are life/death scenarios, or imminent security risk scenarios. Certain “harm” scenarios or patterns which may be recognized by the system 3500 from the anonymized data processing may include more generalized behavioural patterns which have value beyond immediate threat detection. For example, more generalized behavioural patterns may include sleeping patterns, eating patterns, exercise patterns, or the like. Furthermore, it is to be appreciated that anonymized data stored at 3520, which may include historic anticipated harm scenario and/or characteristic detection data, may be fed into any one or both of the characteristic recognition model at 3512 and the pattern recognition model at 3522, particularly where such historic data carries weight for a particular individual or a particular environment (e.g., if an individual in a defined environment is prone to self-harm, this may weight the pattern recognition model at 3522 accordingly). If no anticipated harm scenario is detected, the method ends at 3528 in this embodiment.

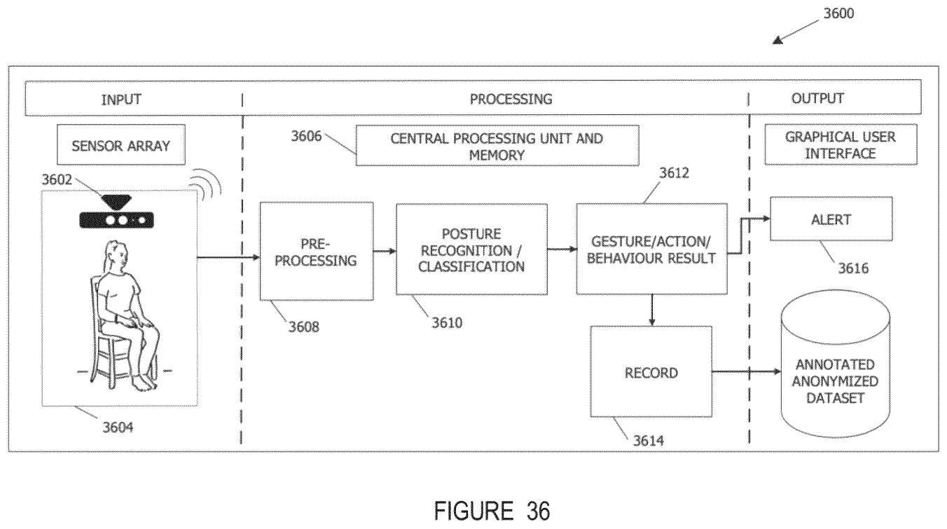

Turning now to FIG. 36, this figure illustrates another exemplary privacy-maintaining system 3600 for automatically monitoring an individual in a designated environment to identify a risk of harm (again, “harm” intended broadly). As shown, the sensor array 3602 arranged within the designated environment 3604, which may comprise a single sensor 3602, in use captures anonymized sensor data of the individual. This anonymized sensor data may be inherently anonymous (e.g., radar or thermal data of low resolution) or otherwise may be immediately processed by the sensor(s) 3602 in a manner so as to automatically anonymize the data upon acquisition (e.g., by performing body segmentation or blurring to provide silhouettes, by extracting or tracking skeletal projections, or the like). To facilitate such immediate anonymization, in some embodiments, one or more sensors 3602 may include one or more internal processors (not shown) configured for such immediate or real-time data extraction or abstraction which yields an anonymized data set (or a dataset devoid of PII). Examples of anonymized data are shown, for example, in FIGS. 6 (skeletal data or tracking), 10 (skeletal tracking and body segmentation), 21 (skeletal tracking and body segmentation) and 34 (facial blurring). In this embodiment, the anonymized data acquired of the individual in the designated environment 3604 is communicated to a remote central processing unit (CPU) and memory 3606, by either wired or wireless communication, and is then pre-processed at 3608 on the remote CPU. As an example of pre-processing at 3608, if the sensor array includes two or more sensors, extracted data (e.g., skeletal data) may be merged or combined during pre-processing. In another example, skeletal data acquired by the one or more sensors may be annotated (e.g., specific joints or positioning labelled) during pre-processing 3609. In yet another example, noise may be filtered out during pre-processing at 3608. In this embodiment, the preprocessed data is then fed into a posture recognition or classification model at 3610, which is operable to detect or classify from the anonymized data certain human postures so as to detect or classify particular predefined gestures or actions or behaviours. Upon detection or classification of a specific gesture, action or behaviour at 3612, typically to a predefined confidence level, the system 3600 will automatically record the anonymized data being acquired at 3614 and will optionally, typically depending on the nature of the gesture, action or behaviour detected or classified, generate an alert at 3616.

In both the systems described with reference to FIGS. 35 and 36, it is to be appreciated that the models therein described may comprise specific convolution neural networks (CNN) as described herein, which are specifically adapted for the characteristic feature(s) recognition (e.g., posture, gesture, action, behavior) described herein, based solely on anonymized data. Accordingly, such systems are privacy enhancing as any data stored is anonymized and therefore any data breach would provide no PII.

Furthermore, it is to be appreciated that any one or more of the systems, methods, or frameworks described above may be configurable as a more generalized behavioral analytics system, method or framework. For example, specific characteristic features detected with system 3500 or specific gestures, actions or behaviours detected with system 3600, or the like in other embodiments, may be recorded on an ongoing basis in annotated anonymized data format for future behavioral analysis and/or pattern recognition. Accordingly, in some embodiments, the system not only detects or alerts to specific events-of-interest, but also continuously identifies classes of actions and/or behaviors occurring and documents same accordingly, using the anonymized data acquired and/or indicators of same. For example, in one non-limiting embodiment, the system may recognize that the individual is sleeping based on the anonymous data acquired. Based on such an activity classification, the system may track the length of time spent sleeping, based on the anonymized data acquired, and record the duration of the sleep activity, as opposed to the continuous data reflecting the sleep activity. Such recordal may further anonymize data, and may advantageously reduce storage space required for the documentation of such activity. Identifying the classification of actions and/or behaviours occurring may extend to any other actions or behaviours recognisable from the anonymized data fed to the recognition and/or classification model. Recordal of these classes of actions and/or behaviours further enables the system, in some embodiments, to receive from the remote device behavioural queries and, based on the data stored, to provide behavioural patterns recognisable from the annotated anonymized data stored. For example, a behavioural question posed to the system may include, “how often how does the person sleep?”, “what is the number of hours spent walking around the room per day?”, “how often does the person sit?”, “what is the general level of activity for this person?”, “has the general level of activity gone down in the past week?”, or the like, and the system will, utilizing stored annotated anonymized data for that individual, provide behavioural patterns for the individual in the defined environment.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described. Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A system for automatically monitoring an individual in a designated environment to identify a risk of harm while maintaining a privacy of the individual, the system comprising:
an image-based sensor capturing image-based data of the designated environment to extract therefrom a digital representation of a posture of the individual within the designated environment such that an identify of the individual is unidentifiable from said digital representation;
a radar capturing radar data of the designated environment;
a network communication interface;
a computer-readable medium having stored thereon characteristic features of multiple anticipated harm scenarios, wherein at least some of said anticipated harm scenarios are digitally characterised by a respective posture and vital sign;
a digital data processor executing digital instructions in real-time to digitally:
process said radar data to extract a current vital sign of the individual being monitored;
compare said digital representation of the posture with multiple predefined postures to categorize a current predefined posture of the individual being monitored;
identify a risk of harm upon said current vital sign and said current predefined posture corresponding with one of said anticipated harm scenarios; and
communicate an alert corresponding to said identified risk of harm via said control network communication interface.

2. The system of claim 1, wherein said image-based sensor comprises a depth-enabled image sensor.

3. The system of claim 1, wherein said image-based sensor captures sequential image-based data over time to extract therefrom a posture sequence representative of a gesture, wherein at least some of said anticipated harm scenarios are digitally characterised by a respective gesture and vital sign, and wherein said digital data processor executes digital instructions in real-time to digitally compare said digital representation of said gesture with multiple predefined gestures to categorize a current predefined gesture of the individual being monitored to identify a risk of harm upon said current vital sign and said current predefined gesture corresponding with one of said anticipated harm scenarios.

4. The system of claim 1, wherein said digital representation of said posture comprises a digital representation of a physical disposition of physical body parts of the individual being monitored relative to said designated environment.

5. The system of claim 4, wherein at least one of said anticipated harm scenarios is characterised by a predefined physical disposition defined by a suspended vertical body orientation.

6. The system of claim 1, wherein said vital sign comprises at least one of a heart rate and a respiration rate.

7. The system of claim 1, wherein said image-based data is digitally anonymized by digitally removing predefined personally identifiable information (PII).

8. The system of claim 1, wherein said image-based data is anonymized by executing a predefined data anonymization process.

9. The system of claim 8, wherein said predefined data anonymization process comprises automatically processing said image-based data at said image-based sensor to extract a skeletal projection or anonymized three-dimensional body representation from said image-based data.

10. The system of claim 8, wherein said predefined data anonymization process comprises automatically processing said image-based data at said image-based sensor to identify a facial border from said image-based data and blurr pixelated content within said facial border.

11. The system of claim 1, wherein said image-based sensor comprises one or more of a colour (RGB) camera, a colour-depth (RGB-D) camera, a depth camera, or a dynamic vision sensor (DVS).

12. The system of claim 1, wherein said respective posture defines any one or more of a body motion, a body posture, an activity level, a predefined action, a predefined behaviour, or a predefined relative body disposition.

13. The system of claim 1, wherein at least one of said anticipated harm scenarios is digitally characterised by a combination of said vital sign, said respective posture, and at least one of a digitally detected presence of a designated object in the vicinity of the individual being monitored, an anomalous presence in the designated environment, or a temperature associated with the designated environment.

14. The system of claim 1, wherein said image-based data is further processed to extract respective digital representations of respective postures of respective individuals within the designated environment, and wherein at least one of said anticipated harm scenarios is digitally characterised by a relative disposition of said respective postures and respective vital signs.

15. The system of claim 1, wherein said network communication interface is communicatively linked to a graphical user interface (GUI) and wherein said digital data processor is operable to output anonymized data representative of the individual in real-time for display via said GUI during monitoring.

16. The system of claim 15, wherein said digital data processor is configured to execute digital instructions to digitally merge said anonymized data to generate a three-dimensional textured scene of the designated environment.

17. The system of claim 1, wherein said image-based sensor comprises a depth-enabled sensor.

18. The system of claim 17, wherein said depth-enabled sensor comprises a time-of-flight infrared sensor or at least two stereoscopic cameras.

19. The system of claim 1, wherein said anticipated harm scenario corresponds to one or more of a self-harm event, a hanging event, a choking event, a suicide attempt, or a fight.

20. The system of claim 1, wherein the designated environment comprises a prison cell.
